SEQUENCE LISTING (8 sequences)

SEQ ID NO: 1 - protein, 15 aa, Homo sapiens
SSSNRKAKNPPGDSC

SEQ ID NO: 2 - DNA, 45 bp, Homo sapiens
  source 1..45: mol_type = unassigned DNA
tcgtcgtcgaaccgcaaagcgaaaaacccgccgggcgattcgtgc

SEQ ID NO: 3 - DNA, 5460 bp, Homo sapiens
  source 1..5460: mol_type = unassigned DNA
gggcttcgctcgccgcctcgcgccgagactagaagcgctgcgggaagcagggacagtggagagggcgctgcgctcgggctacccaatgcgtggactatctgccgccgctgttcgtgcaatatgctggagctccagaacagctaaacggagtcgccacaccactgtttgtgctggatcgcagcgctgcctttccttatgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagattccagagtcagtgtcacaaaaccatttatgttaccccctgttgcagccagctcccttaggaatgacagcagtagcagtaataggaaggccaaaaatccccctggagactccagcctacactgggcagccatggcattgccagcattgttttctcttataattggctttgcttttggagccttatactggaagaagagacagccaagtcttacaagggcagttgaaaatatacaaattaatgaagaggataatgagataagtatgttgcaagagaaagagagagagtttcaagaagtgtaattgtggcttgtatcaacactgttactttcgtacattggctggtaacagttcatgtttgcttcataaatgaagcagctttaaacaaattcatattctgtctggagtgacagaccacatctttatctgttcttgctacccatgactttatatggatgattcagaaattggaacagaatgttttactgtgaaactggcactgaattaatcatctataaagaagaacttgcatggagcaggactctattttaaggactgcgggacttgggtctcatttagaacttgcagctgatgttggaagagaaagcacgtgtctcagactgcatgtaccatttgcatggctccagaaatgtctaaatgctgaaaaaacacctagctttattcttcagatacaaactgcagcctgtagttatcctggtctctgcaagtagatttcagcttggatagtgagggtaacaatttttctcaaagggatctggaaaaaatgtttaaaactcagtagtgtcagccactgtacagtgtagaaagcagtgggaactgtgattggatttggcaacatgtcagctttatagttgccgattagtgatatgggtctgatttcgatctcttcctgatgtaaaccatgctcacccatatcccactatacaaatgcaaatggttgcctggttccatttatgcaagggagccagtactgaattatgccttggcagaggggagactccaaaagagtcatcgcaggaagaagttaagaacactgaacatcagaacagtctgccaagaaggacattggcatcctgggaaagtccgccttttcccttgaccactatagggtgtataaatcgtgtttgcaaaatgtgttatgatgtgtttatattctaaaactattacagagctatgtaaagggacttaggagaaaatgctgaatgtaagatggtcccatttcaatttccaccatgggagagcctaaaaataaattatgacatttagtatctaaggttagaaaaccacgcccacatgctaatatgggtgttgaaaactaggttacttataatgcaaggaatcaggaaactttagttatttatagtataatcaccattatctgtttaaaggatccatttagttaaaatcgggcactctatattcattaaggtttatgaattaaaaagaaagctttatgtagttatgcatgtcagtttgctatttaaaatgtgtgacagtgtttgtcatattaagagtgaatttggcaggaattcccaagatggacattgtgcttttaaactagaacttgtaagacattatgtgaatatcccttgccaattttttttataataagaaaacatctgactaaagtcaaagaatgatttcttatggtttattttgatgaaagttcttttaacatgtcttgaatgtacacataaaggaatccaaagctttccattctaacttaatctttgtgataacattattgccatgttctacaaccgtaagatgacagttttcaatgtagtgacacaaaagggcatgaaaaactaactgctagctttcctttcatttcaaaagtccaagaatttctagtatatttggattttagcttctgttcaaagcaaatccagatgcaactccagtaagtggcctttgctcttttttgtaccaaagagcccagatgattcctacagtccctttcttctctaacatgctgtggttccttaaatatgagtaatttctctaagatataacccaggtgctttgagaagctgcattaaggtgttcaggccctcagatatcacatggtacacttgattagtaataaaaccagagatcaatttaaattgctgataggtcctgtctcagtgtgtggcattgactgttttcaggaaaatagatacagattaatatgagttatgcgtgtaggttgtgtatagattgagaagatagatacttctcaatctagtagtttgatttatttaaccaatggtttcagtttgcttgagcatatgaaaatcctgcttaatgtgcttaagagtataataaatgtgtacttttgtcctcaaacctagtagctgggttttaacactcatggacatggtcttaatcaatggagttaaataaacaaattcagcaagttattaaatctgacatggtaggagaggggagatgtgtcctgcttattaaatgtgttggtccattgaaagttacatggattgccaatttttaaaacactaaagttgaataaaatgcatgaacaatagaaaaatgctgaacattattttggatgctagctgcttggacattaactgtgttatttctgctttgagatgaaaatatatatttatctttgcttattttatcccagatgtgttctgaatatccttcttcataaatcatggaaaactcactgctgagatagtaaaccatgaaatcgccttttcagttggtgccatgtatctgacagttccatcttggaaggtttcaaaattaccttttaaaatgatctcagaagtctgtagattctcaatgatactgaaagctttgcacctctttggtagaaaccaggtctatttagaaaatggctttatgataaatgttgcctcctgagtgataatgaagtgttcctggatattgtattgtaatttaatgtgcttaccacactgccacattttaatgagtcagagaaaaattaatttttcttcaatacaataatagaacaagtagcctattctcttaaaaagtatgtgaaaagaaaattatgaaaaaatatgcatacctaatgaagtattggttttagtaagaattaaatacatttcattgagctttaaagtactttggagaaactttggggcacgttttcctactctaattcaactaaagttataaataaagagaaaaactcattcagaaatcatggattttaaaaatattttactgcagccaagttttcatttcaaaatgtaatttcagtttggagcttttaggcattatgtatatttaaaaaatatattcttcaaaaatgcattttggcatggtgggatggatgttgcaaaagatatccggagcctccagtctgtcattaactgatatggtaaatcacctctcttctttgggtctcaattttttatttatctatatggtaaactcagagatcactccttaggggtgagtcctattgcaatatgaccgacaaagaagacaaaatagcattgaaactaacccatacaaaatatccaactctggattctgtgaataagtatcttgaccataaaaagtcattgctgttcttgtttctaatgtaaatagtgtccattagtaaaagtgaaattcagtcttaagtagggtgaattggatcaccatttacacaagagatggctttttcctttgcttgaataaacattttggatcacctccaaagaatgaaaaccagtagtacgttttagtcatattagtcaggatgagaaactataagatgtgtgtaacatttggaaatgcaccaaagtgagcgtttaaatcttctcattttattgaaaactaagagcagaaaatgtaaaatgctcatgaaggttttgaatgccaaaagatattttagaatcaatttataaaggggtaattcattaattacactttaaaattggaaagtgggataagaaatctaaagtaaaccagcttatctttgaaacaatattattttgaaattggctttaaaataaaaccattcagattgaaattctaattagctcatttgtggagtttgatcacacaattcataatgttgctgctttccattaactagtcttgaaatgcctttgtttgtaaaaataaaataatggtactttcattttataacaaggtgtttttttcaagaaataatccatgctaaaatggatatttgtgatcctgaaatgtttactaagcattgtaaatttatttataactgccatctccaactacatccttatgatgtttttaacaataaaattaaaacaactgttaaactaaaaaccacaccgttttccagtacttgatctctgagctacaatactcactaaatataattttccaatcaaaatattctattctatattctaagggttaatatgtgattatagtgtccacttgccaccatttttttaaatcaatggacttgaaaagtattaatttagatggatgcgcagatataccctcagttcagtcatagattggagtttgcatataataatgtaaatgtatgtcgacactattctaaatagttctattatgactgaaatttaattaaataaaaaaggttgtaaaatgtgatgtgtatgtgtatatactgtatgtgtactttttaaaataggtgtatgtcccaacccttttttatacaggtttgaatttaaaattacatgatatatacatatactttattgttctaaataaagaattttatgcactctcaaaaaaaaaaaaaaaaaa

SEQ ID NO: 4 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 5 - DNA, 5376 bp, Homo sapiens
  source 1..5376: mol_type = unassigned DNA
gggcttcgctcgccgcctcgcgccgagactagaagcgctgcgggaagcagggacagtggagagggcgctgcgctcgggctacccaatgcgtggactatctgccgccgctgttcgtgcaatatgctggagctccagaacagctaaacggagtcgccacaccactgtttgtgctggatcgcagcgctgcctttccttatgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagggaaggccaaaaatccccctggagactccagcctacactgggcagccatggcattgccagcattgttttctcttataattggctttgcttttggagccttatactggaagaagagacagccaagtcttacaagggcagttgaaaatatacaaattaatgaagaggataatgagataagtatgttgcaagagaaagagagagagtttcaagaagtgtaattgtggcttgtatcaacactgttactttcgtacattggctggtaacagttcatgtttgcttcataaatgaagcagctttaaacaaattcatattctgtctggagtgacagaccacatctttatctgttcttgctacccatgactttatatggatgattcagaaattggaacagaatgttttactgtgaaactggcactgaattaatcatctataaagaagaacttgcatggagcaggactctattttaaggactgcgggacttgggtctcatttagaacttgcagctgatgttggaagagaaagcacgtgtctcagactgcatgtaccatttgcatggctccagaaatgtctaaatgctgaaaaaacacctagctttattcttcagatacaaactgcagcctgtagttatcctggtctctgcaagtagatttcagcttggatagtgagggtaacaatttttctcaaagggatctggaaaaaatgtttaaaactcagtagtgtcagccactgtacagtgtagaaagcagtgggaactgtgattggatttggcaacatgtcagctttatagttgccgattagtgatatgggtctgatttcgatctcttcctgatgtaaaccatgctcacccatatcccactatacaaatgcaaatggttgcctggttccatttatgcaagggagccagtactgaattatgccttggcagaggggagactccaaaagagtcatcgcaggaagaagttaagaacactgaacatcagaacagtctgccaagaaggacattggcatcctgggaaagtccgccttttcccttgaccactatagggtgtataaatcgtgtttgcaaaatgtgttatgatgtgtttatattctaaaactattacagagctatgtaaagggacttaggagaaaatgctgaatgtaagatggtcccatttcaatttccaccatgggagagcctaaaaataaattatgacatttagtatctaaggttagaaaaccacgcccacatgctaatatgggtgttgaaaactaggttacttataatgcaaggaatcaggaaactttagttatttatagtataatcaccattatctgtttaaaggatccatttagttaaaatcgggcactctatattcattaaggtttatgaattaaaaagaaagctttatgtagttatgcatgtcagtttgctatttaaaatgtgtgacagtgtttgtcatattaagagtgaatttggcaggaattcccaagatggacattgtgcttttaaactagaacttgtaagacattatgtgaatatcccttgccaattttttttataataagaaaacatctgactaaagtcaaagaatgatttcttatggtttattttgatgaaagttcttttaacatgtcttgaatgtacacataaaggaatccaaagctttccattctaacttaatctttgtgataacattattgccatgttctacaaccgtaagatgacagttttcaatgtagtgacacaaaagggcatgaaaaactaactgctagctttcctttcatttcaaaagtccaagaatttctagtatatttggattttagcttctgttcaaagcaaatccagatgcaactccagtaagtggcctttgctcttttttgtaccaaagagcccagatgattcctacagtccctttcttctctaacatgctgtggttccttaaatatgagtaatttctctaagatataacccaggtgctttgagaagctgcattaaggtgttcaggccctcagatatcacatggtacacttgattagtaataaaaccagagatcaatttaaattgctgataggtcctgtctcagtgtgtggcattgactgttttcaggaaaatagatacagattaatatgagttatgcgtgtaggttgtgtatagattgagaagatagatacttctcaatctagtagtttgatttatttaaccaatggtttcagtttgcttgagcatatgaaaatcctgcttaatgtgcttaagagtataataaatgtgtacttttgtcctcaaacctagtagctgggttttaacactcatggacatggtcttaatcaatggagttaaataaacaaattcagcaagttattaaatctgacatggtaggagaggggagatgtgtcctgcttattaaatgtgttggtccattgaaagttacatggattgccaatttttaaaacactaaagttgaataaaatgcatgaacaatagaaaaatgctgaacattattttggatgctagctgcttggacattaactgtgttatttctgctttgagatgaaaatatatatttatctttgcttattttatcccagatgtgttctgaatatccttcttcataaatcatggaaaactcactgctgagatagtaaaccatgaaatcgccttttcagttggtgccatgtatctgacagttccatcttggaaggtttcaaaattaccttttaaaatgatctcagaagtctgtagattctcaatgatactgaaagctttgcacctctttggtagaaaccaggtctatttagaaaatggctttatgataaatgttgcctcctgagtgataatgaagtgttcctggatattgtattgtaatttaatgtgcttaccacactgccacattttaatgagtcagagaaaaattaatttttcttcaatacaataatagaacaagtagcctattctcttaaaaagtatgtgaaaagaaaattatgaaaaaatatgcatacctaatgaagtattggttttagtaagaattaaatacatttcattgagctttaaagtactttggagaaactttggggcacgttttcctactctaattcaactaaagttataaataaagagaaaaactcattcagaaatcatggattttaaaaatattttactgcagccaagttttcatttcaaaatgtaatttcagtttggagcttttaggcattatgtatatttaaaaaatatattcttcaaaaatgcattttggcatggtgggatggatgttgcaaaagatatccggagcctccagtctgtcattaactgatatggtaaatcacctctcttctttgggtctcaattttttatttatctatatggtaaactcagagatcactccttaggggtgagtcctattgcaatatgaccgacaaagaagacaaaatagcattgaaactaacccatacaaaatatccaactctggattctgtgaataagtatcttgaccataaaaagtcattgctgttcttgtttctaatgtaaatagtgtccattagtaaaagtgaaattcagtcttaagtagggtgaattggatcaccatttacacaagagatggctttttcctttgcttgaataaacattttggatcacctccaaagaatgaaaaccagtagtacgttttagtcatattagtcaggatgagaaactataagatgtgtgtaacatttggaaatgcaccaaagtgagcgtttaaatcttctcattttattgaaaactaagagcagaaaatgtaaaatgctcatgaaggttttgaatgccaaaagatattttagaatcaatttataaaggggtaattcattaattacactttaaaattggaaagtgggataagaaatctaaagtaaaccagcttatctttgaaacaatattattttgaaattggctttaaaataaaaccattcagattgaaattctaattagctcatttgtggagtttgatcacacaattcataatgttgctgctttccattaactagtcttgaaatgcctttgtttgtaaaaataaaataatggtactttcattttataacaaggtgtttttttcaagaaataatccatgctaaaatggatatttgtgatcctgaaatgtttactaagcattgtaaatttatttataactgccatctccaactacatccttatgatgtttttaacaataaaattaaaacaactgttaaactaaaaaccacaccgttttccagtacttgatctctgagctacaatactcactaaatataattttccaatcaaaatattctattctatattctaagggttaatatgtgattatagtgtccacttgccaccatttttttaaatcaatggacttgaaaagtattaatttagatggatgcgcagatataccctcagttcagtcatagattggagtttgcatataataatgtaaatgtatgtcgacactattctaaatagttctattatgactgaaatttaattaaataaaaaaggttgtaaaatgtgatgtgtatgtgtatatactgtatgtgtactttttaaaataggtgtatgtcccaacccttttttatacaggtttgaatttaaaattacatgatatatacatatactttattgttctaaataaagaattttatgcactctcaaaaaaaaaaaaaaaaaa

SEQ ID NO: 6 - protein, 245 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKGKAKNPPGDSSLHWAAMALPALFSLIIGFAFGALYWKKRQPSLTRAVENIQINEEDNEISMLQEKEREFQEV

SEQ ID NO: 7 - DNA, 94673 bp, Homo sapiens
  source 1..94673: mol_type = unassigned DNA
agaatactctgtgaacttattatgtatttcatcattttcaaggctggtaaaatcacacgccttcacataggcaaagagaacttcacattcttgtagtttggaaactccataggaaggagatactgttccccattagctcaagcacaaaagtcccacagtggattctgattggcccatctcatgtgatgtacccattcctccaccaatcaggactggacagatcagggtactaagcctatctccgatagtggaaggaggtgggaaaacagaaccatgtgaatgacagtctctcttgaaagataaggaatagtctgaattctctaagtgaaagtgagtgctaataatggaagaagagtgacaggaaagtgtgctaagcaaataaaaaaaaaaaaaatctaagccgggcatggtggctcacgcatgtaattccagcactttgggaggccaaggcgggcagatcacctgaggtcagcagttcgagaccagcctgagcaacatggagaaaccccgtctctactgaaaatacaaaacttagtcaggcatggtggtgcatgtctgtaatcccagctactcaggaggctgaggcaggagaatcgcttgaacctgggaggcagaggttgcgtgagccaagatcatgccattgcactccagattgggcaacaagagtgaagctctgtccccccccgacaaaaaaaaattcctaaccacagtccatatgtctaggttagatcatggttttcccatttatcaattgagggatcttgagcaaatcaccatacttctttgatcctcagtttccttatttatcaagtagaaaagttaaaaattacctctcttacatgattgtcataagaagttattgacagaatataaactaagggcttagaccaattcctggcttatatgggcaacagcaacctaattgaccaatatttaagggattcgctctgttctttcccctgtgacagatattgacaataaaataatgagcaagggcagtcataattactttaaccaaatagtcaaacaaacaaatacaaaactgcagttgttttataaaaaataggtaattcatgctcgactttatagtaagaggagaattgaacaaagcaaagagatcagggaaaacctccctgaggaagaaaatatagggaatactgagttgagatctacatgataaatgcatacttactgggcacaatgatagggtgaaacaaagtggtccaaacagaggcagaaaggagggtatttgcaaatggcctgaggtgagaattatgaggacagaagggccttggccctggaaaaaagggagcacataatgaattgaccttggagaaggagggagaggcgagatcctggaggccttgaaggctttgctaaggtgctaatttctttattccaagagcaatggcaagccaccaaagagctttcagcagaggaaatttcatgatcaaagaggcattcttctccttttccccctcctctagtcactcaaagacagatatagacagtctgtctccggtctggcttggtcaggcaataagtttcagtttgtgagagacattttttaaaagaaaaatattgaaaacaatatgtgtgtctaaaagaagatatccacaaaatagtgaatgaacaagagatatttcacatgcacaaagcaagataagacttatttggtaaattacacctgcctcttaacaacaaaaacgataaagctcaggaaaaaagcttttcaaaattggattaatcaataatagcatgagtgacgaatagtattgaaagtattcaagtaaaaattagataatcatgaaataactttctacccagtgtgttaaatgttgaattccctgagttgtataatggtagcctgctatttccataagaaaacatcccccatccctcctgtccacctgggacatagccctgcctcttgttcagtttaaaggaactccttggtttttcttctgcgaatcaactgaatgctggcctgcatcccctatcaaaagccagtcttaaggagagatctcagtcattctgacccatttgtgacttccatataagcctacccaaaatctttcatttgcatattagctctttctaatcccttggatatttgcttcgacattctcttgattcaagtaacattattacacaacaaaactgtaatgcagcagtatgagggaaacaaaagatacagttacgtatgagaaagtcagtgatattaagaaagctagaaccagggaatggtggaggtgggaggaggagacagaactgagagggaaaaaaaatggaaagagggagaaagagagaggagaatgagaatgcattgtaggagatgaacgtagcatgtattattgagttgaatgaaaagaaattgaactgaatttctgttaggaacactgccttgtttctttaaacctggtacaacaaattaggccattcctttctattggtctcataaaatccgtatggttagatttatgattcccaggcttcagtaactctcataccattttgattatttttgtcgtatctgtcctcctttagtactacttgttatttacttaatgttatacatgaaagtgagtttatattgacactaaatatttcttttcttggggggaaaatggtggtaaaggggtacaaagttttagttagacaggatgaataagttctggagatttattgtacagcatgataactacagttaatgtactgtatacagttaacccttgaacaacacaggtttgaactgtatgggtctacttatatgcaatttttttttcaaactcagatggaaaatacagtgttctagagatgtaaaacccatgtataagcagggctgacttttgtgtctgcacgttctgcagggttgactgtgggacttgaatatatgtagattttggtatacctgaaaggtctaggaatcaatcccctgaatatactgaggtactccaaaactacaaagagtagatgttaaatactctcagctcccaaaaatgatgagtatgtgaggtgatggatatactatttagcctgatttaataatttcacaatgtatacataagcagaacatcacattgtacgtggtaaatagatactatttttgtcaaatataacttaataaagctggagaaaaaaaataaaattctccacttcaaaaaagccttaaaggggccgggtgcagtggctcacgcctgtaatcccagcactttggaaggctgaggcaggcggatcacctgaggtcaggagttcgagaccagcctggccaaaatggtgaaaccccgtctctactaaaacaacaacaacaacaacaaaaatacctgggcgtggtggtgggcacctgtaatcccagctacttgtgagactgaggcgggataatcgcttaaacctgggaggcggaggctgcattgagccgagattgcgtcattgcacttcagcctgggagacagagcgagattccatctaaaaaataaaaaataaaaataaatcttgaagaaaaaaaaaacaatcatttgctatgcctccagtatccaaattaagctcattttgatatttgatgttcgatatatttgtactaagagtacctaatgtataaaaatctttctaagcaactatcaacttggaatcatgattgatgtgctgtctatacatattttttctaacatgagaaagtgagaacgcagtcccccactaaattatgcagtcgagtttcccacatttggggaaatcacagaagtcagcacatctggagtgaaataaacaagcttcgccctgattacaggtttggcatattcatgtttaacaaacgcaccggaactaattaaagcaaatttggactcctactctattattaaaataactaattcctaaactcatacattattctttcttttaaagatcactgatgttaatgttcagctaatattttcttttaaaaaattaatgaaatgctttggaatgttctccagttatacaaacaaaaacagacacattctggagaatcaataaaatatttgaagtattttttaagactgtacagctggagctccaggtttaaattagagctctgccatttactagatatggttggtcaagaaaattaaccttgtatttcatcgtccataaaatgggaataaaataataagatctacactaatagatttgatgaaagaatttaatgagataatctatgcgaagttcttagtctggcacctggcacatgaataagtgcatcacataatgatgttagctattattattgtggttgcattatctttccgttcaaaaaagtacaactttcaaagccttgctgtgttggcgacctgtagtttaataatctatcgcttctctttcaatcttaatttctaaagacacagtaacaaaccattccaaatgaagcgtctccccttgcaatacttaaccttgagtcaacaattcttgtaattattaaatattcattcactaaagtaattgttttttttaaaaaatagaggtgataggacaatggcctaactacagatcgcaattaaactttatttctctaaggcaggcagggtgaattctttgctccgaacctcccggactcctctccctctccgactttgtataacagagggagctccgagccctctctggcgcgcgaggtatttcgtctgtccccgggggtgccaggtgagccccagcggatccgggagggtaagctgggactcctcgcgagcagtagctgcagggtaccaagcttcgccctctgcgtccccgcgccttcgcggtctcccgccagtgcaggtccggggcccccaggcgagcggacaaggttggcctaatctgccaaacttctggggcatttaccgtgctctggccgccctcccgattcttccctccgcgcccttgcctgcttctcgcctaccccgggctccggaagggaaggaggcgtgtccggagcaggcgggcgggaactgtataaaagcgccggcggctcagcagccgggcttcgctcgccgcctcgcgccgagactagaagcgctgcgggaagcagggacagtggagagggcgctgcgctcgggctacccaatgcgtggactatctgccgccgctgttcgtgcaatatgctggagctccagaacagctaaacggagtcgccacaccactgtttgtgctggatcgcagcgctgcctttccttatgaagaagacacaagtgagtagggcgcgcccgggagctcccaggctctccaggaaaaatcgcgcccggtgccccggggaagccggcgctccctgggacttgcagctggggcgtgcagggctgtgcctgccgggtgagacaagaggatgcgggggaggccggcgtggtgtgtgatcccgagccgagccgggtgagccagggagaaaaggagtgggagtgctgagagggagccagtgtcaagtttggagcctcagcagttaagttttgagctgtcagtcggaaaccgtaattcccgtctggtggaaagattggcttttgggccatggaatgttaagttatcacgggaaggcatgtctttgcataggagacggttttcccgtggattccaggagattatgaaacagcaccccacgcgccaccctcactagctctctcactcagatttatttgtgtagccccgaggtgggcgccgggcaccaggagcggcgcgaacgcgggtgcagccgcgggagcgaagccaacgcggaactcctaagcggcagctctcggcggggcgggaacctgcccgcctgcccgggaaacttggctctcggcggtgtctgcctagtttgcagctctgtgtgggctctaaagagctctagtacctccacaatgcacgcacctttttccatttcaaaagggataaaggctttttataaaataggcacctctgaatgatagtgattgagataattcccagtgctgctaaattctgcattatacataaagcactgtgggtaaagcgctgcctggggggacaattagatttttttttcccttgaagaaaatacctggttcttcgtttagaaagggccgccacggtgcccctatccacagttctcttcaccattgttgattgttacctcctctcactttcccccttagaaattgttatgtgttatctgccgcggttggctaagggttgccctttcaccccccagaggttctgttggagattgcagttcctgaaatgtgtggaaggggtggaagtggtggggaaaaaggaaaccggggtgtttgtacacatcgttggtgtagaatgaaggcatcaatgtgctcaaaatgctttatcaaatgacctgcttagcatcatattttaaacagatatctaggcctgcaaaaagttgcaagactctagccccttgtaccggtgtggtaaaaggcaggggaaagggacattgcctggctaaaagaggcttgcatagtctctagagcactctctaagagaaaagacgcgatgtgcctctcccctctcttacaagcaggttagctttcaggcaaggcattgagcaccgcacctggaatcaccacagcgtgctgtcgaaatgcaaatgttaaagattcttgcaacaaaagaaaagttttattgtaatctcttgatttagacaagctttctgcgaaattataagaagggtagagacctattccttcttatagtagcatctgctcccatggatggtggtgagatctgggggaagaagctgctaccttctagttcaggaaaagagcctaaaccttcctatttaaacaagaacacaggagacagataaggttaatgtccaacactccaaagccaggggatgtactatcaagtcactttgagaggcagtaggattgattatccttgcacccttgctttgcaaaacaaatgtgagctctgtaatctcctttctcgctttgattaaatggctgtgtaaatcggattacatggaataattccattgaaaactagaaagaggtcaatctagtctgaaacattgtgactgtggcattagtgactcagtgctgccgaccagagaaatttgagaaagctaagtattttagatatgtgacctgttcggtcacacatcacacaagttaaagtgcaggccctttgggggaacttgctgatgttaaacggcttgtaggcaaaggagggcagtacccttgttgaatgaaggcattcattgcctaggatctaatgaccttcaaggttttttttaagaatacttctagcgattccacagtcactgaaaaaggggacactggaaatagcccaaaatcaataaaataatctgcagaaatggaggaggggattagatctttcatattgttaaattttctaatccaaaaattacaaagacattttctgtgcaatgctgcatggatggaaggtagcctatgtaaattggacattaagaaataggatcgattgttctgattctaatgttatcctcaccagggaagaatatctattaatatttagtagtttaaatagtcatttataaacatggcatcaataccatggttctattataaagcaacttgttctgtactctcctactaccccacatataaaaaaaatttatagtaatattttaaaatggggtttgatttgaattacacagctctccaagaatcatggggactccaggtattctggcctttccgtcaagctaagtaagtgtgaaaattccatcaacaaccacttggggtttgatgaatgattcattcatttagcatgttggtacacttcagtatttccaatataatgacacaggcaataaatttaagcacagttctacagaaatgaatgtcttttgaatttttcagatttctgtttgattttattttattcagattgtgtggttttacagttttctagaggtgtttgtggcttgggcttagaaaaaacatctctttcaatgtgtaactattttttaaacataatattctgatacaccatattggataacaatcaaggcaccatattttgcaacactgattaagatttggttaaaagtgccattagcttaatgtgcctggtgttacatttatcaatgttgtgcttacattttagtttggggatggtttgtgagaagtgtcataactcctgtcaatttcagaaatactgagataagaaagattgatcttgcaaacaatgtaaaaaaggccattttaggcatactaactctcagagaataagcaaatagaatgctaattttcactactccaaagaaaatatctcttttatgtctgtatgaagtttgtcatgtgggtgatgatgcaagtttactttgtagaagaaaattaacatacttatgaaaagagcatttttttagtcttgtccttaagatcactcaatttggaatgtatgctttgaataattccaatttatgtagcttttaattgatacattctgccaagactttaaaacccaagtgctaagttgattacatttaacataaaacaaggaaaataaccatatggaaatctgaaagtctgggccaaaccaattaaaattgctaaattttatttacgtctaactgctgatttgaagatagcaaattctgggtcacattttttttttaataataggtgactgaaatgtttccatcaccataaatcacataaaaatttctcactcaaatagcaagtgtgggtattggaggttttttacatggattggcaaattccactacatgtggccttcgctttatatctgagaataaaccatatctgagcatatatgagaaacacctaaaatatggttggtagaaggattctaggttctagcttctgaagacttagattctaattcctgttttactactcatgagacaaatgaccttaggcaaatatttgttaaactttttgggatttctgttttttgtaatctgtatgatgattataaaagttataaagatttttctcgcatggtggttttaaggactaaatgaaattttatatgtgtctttataccagcatagtagcttactatatgtacagtttgaatttattgttcctgagaagagagtgcctgctggcctacttttacaagccagagaaaaactgacttattgcaggtaaattatgcctaatgaaccaagcacagagaccaagttttcctaaatctacgtctgtcgatttggtgactcccatgataggctaatatctgtttatagactataattaagacacatcaatccaggaatatgtgaagacaaaggtgatatgtgggcttaagaggtgctacgtttttgaaggtttttcttttaaagtacaaatgaatgagataattagaatagaaaatagaaatggaaaatggaaatattatctttatcgctgactcagtttcatatttctacactcttgctaagttctccttgtatgattctaaggaattatttcattttattttattttattttggactaaagagacttggttagtttcccatccctaatgttctcgcacttgcttttctgagtgaggccatgcattcatttctgtggggtgtctttgttgtctttaccaggcaacgtgcagtgacatttttttttcattatgctagtttgagacttttatttttcttttctggattatcttaatgttcccttttgtaaattatttttattcccttttgtaaattttattcccttttgtaaattagggcaaggcatttggatataaatctctgaagagtgatcttttatctttctttctagcacataaatcatgagcagctgcaagaaactgttcttggtcttacacactgttcttggtcttagtcacgtgtgtagacagaacaatttacaaaacaagttgagggaacaccaagatatttttataatatatgctgctttcagggttgttttgtataggctctgtttcatagaaacaattgactgcagcctttctgatttatagatgcagcaagatgtgtttgccttttcacttaattatgggtatactaaaaactgtttacaggggctgggagtttaagagaattgtcatgatctcatctctacctatttctttctctatagaaaaaagaggtgggggagtgattttctcatagttggggctcatggttctttctttaaaatcataatgtgtgtcatctaaaggaagactgttctttatgtacctgtctttgtcctttggctacattttactatttttgtaggctgccactttggcttggatattaggaggctgatttcttttattaacctggataataagtagtagtaatttgggaaattatttgaactccagatcttagtttgctcaactgtaaaatgagtaagtgagactaggtgatttctaagtgccttctggttctaaaattctctatttctaaagtactagtaaaatagatatttgaaccagatggtatttaagcttccttcagttctcagatttatgcagccattgcttcagtgtgaatgcacattataaccattgttttatgattatgaataagccaatggggagaaatgaaggagatttaaatttagactgttctacttttatgcatttcacctactgcttgatatttctttgtaaaaagaatggtatttacactttcctgttttagcaaaatccatagttaaagaggatctttactttctctgttctaaatttttaagttctatgaaaaagaattctaagataaagtcagctaaccagaggagataagcttacaaaaggtaatgcttccctgtatttgcctgttttttttctgtccctttaacttccagtacgtgtgtgcatggagttgaaatggagaaagaaaaccaagaagggatctgatgatgtagtagaaagagcactgaggggagaatcagaggaactgatcctaccctttgctctgcctcaagatgtgtgaccttggactagtctcccagtcttagtatctctgaatcatgcccaagatagttaatcctgagaagatagtgaagatgagaggctgcagctggagacacggaggtggaaagaggagggagcctcagggaaaggcagttcctgcagatgatactatgccaaatctctccttggctggcttttgtctgggtccagtaccatccttggaacagtgcttgcttacctcctttccttagctgagagctggagcaaacccagcagctagccgtagcccttttctttttttctttttttttttttcttttagcagtcagaagctatggcagtcttttcttgttgacatgagaatattccaagatcagctggaccctcctcaagctaggagaacacacatttctcagaaaacagggccacaaccatgaagataatgttcttctctaatcactggcattgcccactgagctatccactaaaacaaagtttagaattgaggcaatagtctagaaaatttaaaaaaaaaaaaaagtagcaatggctattattgggatatttggatgcagaaattcagcacttgactttgtgcctgtagctgttgccctggggctcttgaatgctgctgattagaacatgggtaaattgggagggagcggggcaataaggtagccttttctgtgttccaggtacacagacagcagctcaaactttgtctgttgctcacaaagaatgcctttgctctttagggtgaccatgtaaaaggacatgaaaggaccaccgcaaactcatcggtgctcacttgaaaaagcaagccttgttcacctttgcagaaagggcatccccactgcataattgcatccctgtgcaaacctgggaagaaagagcacaggttggaggggggcgttgtgtttgtcttgtatgcccatactgtgttgccttttcaggagcagaagttcatctgctgaacataagccctgtgagtgcttagaacttttccctctttttcaccttgacataaaatccctaaagctgaggacctttccctttcaatgtcttcactctgctccagggctgaattgcatctcattattggcttgtttcacctaaatgtgagtatttgagtggcagtgaaccaggttgcctctaaggaatacaactgaataatggtggtctattgtgaaggaacctcttagggccctacagtgctgagttttctactgtttacttggagttcctttctcagtgccttcccaaacaactgccttgggaaaacacacagattctttggcttccaagaagagtgtgacagagaaagtgtgctttgttataaaggccttgtcaccctcctggatgtgttatccatatcgatgtactggctgttaaaattcatgttcactgcagaactctagatggaggagtggtaagaagggagggtagagatgttttcatataaaattgtttctaatgtgatctagaaaacccaaggattgcaacacagacctcagcagtcatacatcattatttatggttctgtgtaaatgtacaatttaacgcttctgaaatagtaaacacattagggatttccttttttgatgaacaaccaaaagctctgtgattgctttagtttggatttctcgcaactttagaaaactgctgtattccgtgggccctactcataaatctttgatgtgctctgttagtcagaggactgaccttagcacatttgtcattctgctgtttgaaagatacattatcacagtataggagaatggctggtgctttctacttccaggtggtccaaagtgggaactaaacaaaatgaggggataataaagggattaggcaacattcatgaaaaattcagaggaaattattagcataatgtttttactatttggatgacattaattgtatgtagaaaagcactgaactacaaaatagggcttacataattcttccaatatctttgtaacaaattatatatatatatatatatatataataatgtatataatatatatataatgtacatatttatatttattcttagcccctgcccagaaaagtgtttttaaaactttacttcctgtttgtaatgtactgaagccacagtactctatgctttaataatactttaaaaataatgtacctttttttaaaatatgaaactagggtctttaagaaaaaaatgtatttaggcatttattaggcttgcttgatttatttcatatatagatacacatatgactgtagctaattttaatttaaaattataagtagcttctaataatactagcaaatccttatataatgctcacaatgtcaggacctgttctaagccgtttgcatttatttatttatatttttcaataatgagacttttaaatccaagactgatttgaccatttattgtttgcacttctgtacaaatgtcacacataatgattcaacttgaagagatgaaatttactgtactatgtgtcaagtgtttggcatttaaatgtgtagccattttaaatagcttctatcattatattataaaatcaaaccatcctgataatagatcatgccaaaatggcacttagtagaaaacagttccaagattaattaattcacagcacttttccgctgagaggtagttattatttttttaaaccccatttcacagataggaaaactaagacaaggtctttctcctgccaatcccaggtgtttattgcaaaagagactcagctggcattaaccacagcgcattagtgtggcttatttaatacctcaaagatatgcattctgtttacatgaacattgaatcaaaaacaaatgcaactcagatgttcaaaaaagctgccagtaatagaatttggacaacaaaaatttcatgattgcagttgcctcaaagagtggctcctgccaactgctcagtaaatacacttggaatgttgtgaaatgaatggagacctaattcaggttgttagatactgatacattttttataagccttttttctaatggcaaaattttaacaattgtgctaggtctgttatacacacaaatgtgtttggtgcatgactggcaaggctactttgtttactattttacagcattaattttcagtgaatgcataacatcatttacttagtgaaactggagacaatgtgagagaatagcaaaggtttgaatggatatagatcaaatgtagtattggttctgccacttaccaaccccagtaagtttttctctttgaacttcactttctcaatatttaaaatgggcacagaagtaaccacctagtagtagtattgatgtgataattaaaagagatagactgtctaacatcacagcaactggcacttacttaatagagcattgtttccttctctaaaagaatagtaaatcacacagaacattatacaaatctgtatgctgtaagaaataacttcagtgttagtaattcctggctttggctatagggctttcataaataaaaaaattaaaaccaatccttagagactaatagcttaactctcttatttatacgttatctatcttatttgacactcataaacacccttagcaaatttttagaaaattttcccaattttatcttctttacttttctcccctttctgttgaaaagaaataacaaagtaggccaaagacaagctgtttaagaagtaagagcagcttactgaataaggaaatataagacaaattaataagttaataactgtgaattactccccatgggtgaaacaccatgccacaaactgcacaagtgtatctcattcgattgtagcaaaatgttgtaaaatacatattattatttggcatcattttttttaacaaagaaaaataaaaaccctgaagttcagagagattaagaaaacttgcccaaaatgagaatttaaaaagtgcaagtggaagagtttgtatttgaagccaagcctatttaaatctaaaggccagattccttctaatcactacaatataatgcaccaatatagacacgcatgcatacacatacaaactgaaagggttgaacttctttgttttttttttgttgttgttgttgttttttctaatttatttgtggagaagaagatgcaatttaactttacaccagaaagactgagagcaggcaaagctgagtcctctggttgagtgatgagttgcttctgaccccacactcctcagtacttaccagaagtgtaaatggctgtatgtgagcaacactcccacaggtcagggagcctgggtttcctcatcaggctttcccacccatctccccagcagtcaaatggaaaccccttgcctgactcatctgactgttgtgagggaatctcattgtaaactctaaagatgtcgtcttactttatggaatcaggaattacttcattgccaaaaagctttctgtctttcaaataaagtggtactttgttggcagagggcagattttaaaatattttaaagttatgtttcaaaataacttcttggaaatagaaagatacttcagaaagtacttttatcaatttgctctgatcctctttacatgatgttttggaagatggttttaaatgtctgtgatcatattaggaaattatttgataataatgtcgtttatgtctggcttttaaagctaccagccatttatattgtggcctttgttttgttcatttcttatcaatccaactgcattaatattaaaaaaaatattttctgcattaatatcgcaagattttaaatgggtgattgtcagcacaaccttatcagcagcatcaccttgtaacatactcaatagcagtttagtacaaatgaaaatgttggctaaaaagtcctggcatcatctcaacacaggtaaaggagacaacagaacagagatgtgcttaaccaaactctgcagtcagatggtctgctttggttgcctaagaacgaaaacctcttagcctcaatttccccatctgccaaacagaatgagcaatggtacatttatcataggtagttataaggataaaatgaaataatccacaaaaaagcacttagtccagtgctggacaccaagaaaatactaaataaacatttactcttgctattaccattaaacaaatggtaatgtaatgcttatcaatagtgatggctcctttcttaaagaaagtgactttaactgcaaaatctccactaccagagtgaaagggaaagttgaagcaatttatccaagtaactccagcctcaaattcacttttgtcctgagtaatgtagttgtttttattctttttttaaaacaaaaactgaaaaagaaaaatgtgtataattagaatcacaaaagagtttacctagagcaaagttacttctttctatcatgtgcccaagatcgcggcaccaatatgtggtggctggcttatgaaacagattcggattcagagctcgcattcccaagctctttgctttacctggaattagggggacagaacatgagtcattcccattttgaatcagtgagtgattgcctacttttccctatatgagtgaagtcattttcagcatccatagaaatatagccacttgtcaaaagaaaatggaaagaacatggcttttgtgtcaaaagcatatagattttacctctggctcttgactctagcttgacatgggaccttaggcaatttatttatctttccttaaccttctcttaaacttctgtaaaatggaagaataatactttgcagcttccttctcttgtttcctcactcttttttctctcccttctttccttgttctttcccttcttttttttaaaaacctggaaataatgttatatacctaatttgtaatcacgattcacaatcactcaaataattgtactattagaaaacaatgtttttcagaattaacacggtttcctgctctaattttaagaagtaaattatatttctcaaagtgtttggctcattggccttgtgtagactggaagaagcatggggtttggagttagacagaatgagagttaaatcttagctccactattatgattacaacatgatatttatttccttcctctaggccccaatatcctcattggtgtaatggggttgtaggggtaatagtacatagttggaagaacacaagaattaaatgagattatagtctgggcatggtggtttatgtctgcaatccaaacactttggaagacagaggcaggaggattgcttgagcccaggagtttgagaacagcctgggcaacatagtgagaccttgtctctacaaaaatattataaataaaataaataaataaataaataaataaataaataaataaataaatgagataatatgtggaaattgtctgacccagaagtgatgctcaataaattgtatctgtaagctttagggaatggattatgccagcagacacatcagtatgtattagactgtctgctctgtgtctgtaaggatgttgacacatgttggcgacctcacaagttgtgaaagtcaagagagttaattaactgagcagtgctctacatactatactgtccattcagaggcctagtgttgttgtattgttctcagacttgagcaataaaaaatataaattttccaagtacatgaaaactttagtttattaatgtcttctaaaatgagcctcaaattagtttttgtatagtatgatcatgaatagctttggagactcataaatctggaatcatctcttcaaactgtatgactttgagtcacttgattcttcggataacattctgattttctcacttgtaaaatgaggtttataatagtgtctaagtcctaaggtgactgtgaggattaaatgagctaatgcataagaagggcattaggacaggtaccagaaaacctcggtaaatgttagctcctttcaaataaaatgcagttttgcattcaaaggttgttcgtttgttgtttttattttttttgcatgtgttgaatttaataactttatagaatcaacagtttggaaatgtttccattatgaattcaggttttattttcttgtcagtgactctgtttagtttaagggcttggttgtccagattcatctttgagtaagttttgtatcaatcaagacagatgcaagttacttttatcagtccattacttcaggtaaacaagcattccattctttgacagaacattaaactgttatgttctttttgatggagtgctgatttgttcaaaacacttgtgaaagagagaaaacagctcctgttttataactcttttgtaatacaaagtttgtttccaaaggttttctggaagtttcttgattgaatctttgattgtggataccacagtgattaatttaccaagccggaaaatgtgggaagtaattttgggttcagttcagagtgggatctaacacaacactttgaaccaatattaaaatgcagaaatctcttttagatattgaaaaagaaaaacaaatatatatatattaaagtccctgcttttaaggcctaaaggagaatgtagtagaaacactgacatgtgtaatttttgtgaattgcttaggaacagaattagatttaaaaaggaattccttttgcacttttccctgaagatttgtgtcctagcatttgaggagtaatacaggaaagtcagaacaagaacttactgacagcgcaggagatccatgtcctaaattcatgtatgtggtactgtattgctatgcagctttcaggatgtccattttattgtccatcaaaatgtaggggtagatcctataattttaaaggttctttctaatgcacacaatctagagttattaacagaagtctgactgtgtcattaccctggctaaaatcctctaccgactttacattaccctcaggataaagtccaaaccctcagcacactatacaaggtcctctgtggtttggctgttgcttatctccaccactcccgagtggtaagtttcattccagcgtcacctaactatgtacaatgacataccaatattgctgcacttggtttccaaggccctgagggcctcgcatttgggtagctttgatggtctctactccaggaagtcctccctgacttttgtttgctctttttcctcagcccctctccctactatgttgtttagtgatcttaccctgagctcctgtagcatttaaaaaaacaatcatatcacttatcacattatattgaagttatttttttacttacctagctaagttgtctaacactggtatccctagctagtggcacagtgccaaaatattgataaaggttttctgaactaaactatgaatttaatgccaaatgtctctttgagggacaacatccacctgtcagcattctaagaatggatccagacatttgacaattttgtgcctgctgacctcagcagacacatgaaactgcatgtaacactgtcctgtataaacatacatacattgttcaaacagaattttcctcagagagagaaatgataaatgagccactttgggtggtagaagagaatggagacagtaaattaaaaataaataaataaataaaggagaaggtgaggccattgcttgccattcttaaggaaatgacacaataggctgaatgacaagggccagttaaaactcgctaatgccagatatatgaagacttaagaaccagaaaggattttagggcagaatattgttgatgatgatgaagaggaaaaatgttgatttccgatgcttattctctgtgccaggtcctgagctaaatgtgttacatactttatgtctcctgattctccccacctgtgagggtggtggtagtgtgattattccatttgatacaagaggaggaatctgagctcacagaggttgagcaaatttctcagcatcttacaactctcagtcatcaaacctgatctgtttgttcctaatttcttgcttagtattagtactgttgacattttattttctcacctagagattttgtttgtttgtttctttgtttgtttaaatggagtctcgctctgtcaccaggctggagtgcagtggtgcaatctcagctcactgcaacttccgcctcccaggtttaagtgattctcctgcctcagcctcctgagtagctgggattacaggcacgtgccaccactcccggctaatttttgtatttttagtagagatgtggtttcaccatgttggccaggatggtctcgatctcttgatctcgtgatctgcctgcctcaacctcccagagtgctgggattacaggcctgagccaccgtgcccagccgagattttttttgatacataataattcatgagactattacaaatatggtaaatggattcttccttcccagctcacccacactcaagatgcttcctttaaaattctgttaacaatttgtgctagttaataggtagctagtgcaatactcaagtaaaggatgttgtgtagttttggatactgagatgtatagtctttgtctaaaaggagttgaaagacagaaatatgacaatatgagagggtttagatgattctattagaatgatatttatgtatttaaaacagatatggttaaggagttcagcaaaaataatgtctcaatacataagatcagagattaagcgtggaggaagaaagttggagaaggattaacaagaagtgatgttttcaaatttcatgtgtggttagaagccatcatcttagtgactcaaggcagtaatactactgctttacaatacagtgctctaaaataatgttacagatattgcatgtacttttaaaaacatatatcattttggggatactctttgatgccaagtttaaaaaattctgaagtgtttaaaatgatacaaacttagagcatgatctgtgttccttttttttttaatcaggtaagcttcttggttttgggtgtagatctgaaaagttaacatttcttgaaccaataaaatgtttccagttttatataattatatcacaaagtctaccttctgccttgatttgcaaagaaaagctccaaaaaggaccataaagaagaggagataacacacaaatggtgagcaatgaagtagatgtacaaaatggtttgtgcagatttggcacttaaaacatacagcagccgaagtacatcttaaatttcatttagtgaatatattgctcacgagtccctctcttcatttcctgaggaagtagatccctattctctcccatatggtgagaaaatctctttgctatccagtttccctgtttacatttgttcttccatccccttctgattcctcagcaaagcagctgctttgaaaagaagctcaaaggattaccttgaaatggttacccactctcccccttttctgctccaagaagaaagtttctttctgtattttcccagcattattaagagtcaagacttgaggccttaaggtacatttcctatattcttcttttgttttaaacttgaaagcaaaataatcttcccaagtctaccatgaggatgaatttcagaatgcacaataacttacatttaccgagaaaactgttaagtcagaatcttctaaaatagagataacaataacaagtgtgatgccagtactcctatcctgatacattgtaaaaaatcatctccttccttactgtgtcctacatccttttttcttttctttttttttgagatggagttttgctcttgttgcccctgctggagtgcaatggtgcaatcttggcttactgtaacctctgcctcccgggttcaagcaattctcctgcctcagcctccctagtagctgggattacaggtgcccaccaccatgcccagctaattttttgtatttttagtagaggtggggtttccccatgttggccaggctggtctcgaactcctgaccttaggtgatccgcccaccttggtctcccaaattgctgggattacaggtgtgagccactgcgcccggccacatcttcctttcttaatgaatttagagtctcctcctacatcaaaatggtattagagagaatgctaaggataattccatggatctcacgataaacaattggaataaaagagcttgccacttagtcatgattattagcatgaaaatgtgatgatgcagcctattttgggggagggatgaagtgtcattgaatcagctcatgagtagtctggcatgcgactttctgtttcttgcaaacaagttgcatgataggggaacatcaacactatgccagtgtgtccctgaagaaaaggtagctggttttgttttggtcaaagtaggaatgagtctggaaagggattcagttttatgaacattgttttgcctgctttagaaaagatgacagatagataagagtcaaccaatgctttgggaagagataagattagcatttaaaagcttaacccatgtcatcacttctgggtatgtcttccctgagtgaaatcttattctttgcatgcattttgaatgctgaatcaccatgaataatttttatgaatggctactttagaaatgttaatggattgcaactcattggggaaataaaaaatttatggttgcgtattacaataaattctaaataaagcagtatatggaattgttgacttctcagagacttccagtgaacttactgcaggacagtgtctacatttcagctctgtgtgatactttcagtgttttcgcttttttaatatgcccctttttgtcaataaaggaaagaaactccactttatacttacgtagtttcttcagaaagggagctctggggattttatagccaggtcagcccttcaattttgggccacatctaccaggagaagcatttttcaatgattgttcattcattaaaattgtctatttacgttgtccctgcccttcttggagcttgtagtctactcatggttttatagcctagttttccacttacatggacctagggaagtattaaaggtacaaagaaaatggttaaaagttcaaactgagaagcatttagctaatcgtcactatttgaatttagaagttattgagcctaagagtgagaaggatgacaatggtttatacccaactgatgaacaaaaccctcctgtttatccttcactcattcattctaaatatatttacgaggggcttcttcacacctgccattgccttagtcagtctgacaaaacagggagggactgaaagtccatatggcaccttatggtgctcacagtcaagtgtaagaaacagataataagcaaaactggttggcgatgttgcaattctattgtgtaaatatggaaaccagctgctgaggattgaattgtacattgtccagccttcatctctgccccattatatgtacctgggtttattctctagtaacacaatttagatagtgccagtaccttggtctgtaccatataaaagagaattttattgaaggagaacttttatgagcaattcagtagcagttagcaatacatacaatccacaaatatggaggattatgtactatgagaaaggaccaaatcaaggtgctagaggtaagaatgacggataagaataatataatcctcattcttataccatgtatattcttgttgggaggaacagatgataagagtaaactaattgggtaatcaaaataatttctgagaatgaaaagtgcttaagggaaagcaaaactgtgatgtgatgtgatgtgataagaggtgctccttaagacaggttacctgggaaggcttctctgagaaggtgatatttgaactgagtcctgaatgatgagaacaacttgccattggcaaagcccaaaaaggagaaaactggcagcagcaagtacaaagggcttgaggcaggaattaccttccaggtatttcaggaacataaaggaggccagtgtggctagagcatggtgggaagataaaggttactaaggcgtaaacatggagttggacaagtctgtcttccgaaggtctctgtaggccttgattaggagttggagtcttactgtaaatgtaatgggaaggcattgggagattttaagcaggtagtgatcatgttttatatattcccagaagaagatactattgtgagtaataagtctgaaagcaaagaaggctatttagaggatccctgtaatcttccagtaagagtgtgctaagagcttagcacaagctaaagttgtacaagatgaaatggagagaaatgagttttgagatttattttgctggtagaaaatatgatcagatgtggaaataggggactgggaagaatcaaggatagtgcccagatttttggcttaaaccaccattcttcaagtttgggcatgatgttactaggaatgcttgttctagacagcaatgacaatagccatagcaaacatttataaaggtgtattatttgacaggtactatactaaatgcccacttacataactatatttaatcctcagcaaacctataaagtcagttctgttattacacccatttacagatgaggaaactgaggcccagaatgataaaataactttccaacggccataaggattgtgtcaaagtagaaattaagatatgggcagtctgattctaaggttgatgttttccaaccccctcctctataccacttctctgtgcatgagtaaacacactgttttctttctttctttcttttttttttttttttttttttttttgagacagagtctcgctttgttacccaggcttgagtgcagtggtgcgatctcggctcaccgcaacctccgcctcccaggttcaagcgattctcctgcctcagccccccaggtagctgggactacaggcgcatgccaccatgcccggctaatttttttatttttagtagagacggggtttcactgtgttggccaggctggtctcgaactcctgaccttgtgatccgcccgcctcagcctcccaaagtgctgggattacaggcgtgagccacagcgcccagccaacacactgttttctaaagctgttttggatatattagtgaagctgaagttgacttttgaaactttgctatttaggttaacattcacctgttgcccaaacaatttttgcctcacttttatactaggtagaaaagagcctgcctgcttggaagagcggatccaaatccaagctcagggggcagtttcacaacctctttatttcactttccagggctgatctttaatctatggggaggcagaggataaatgagattgtttacaattattatgtgcaattaaaaagaaaacacattttttaaaaaatgggttgtattgtgaatgattgtatagtcatttactgttagcaaagcttttagaatatttaagggatcaataaagataaaattataactgtagcatcaactgtggtttaactgtagctaactgaactagctatagttaactattaactgtaattaacaaaataataattagagcattaatagtcatgtaatgttctcatagatctgccaattggcttgtgagtgctttacataacattgtcttatgtaatcttttcaaaactgcaaaaacatgttatcatcacaagaaaactgagtctcagagaggttacataatttgctctggattacaacacgtgacagcaccaggatttgaatccatatcttactttgataacgttctctgttttaaatgtcatcatcttcataaacagtgagcctgcaattgacatatattgttatcagaagaacaagtcctgggcatgtcattgaacctgcagttgtagagtattgttaccagagaaatgtgtctcaggcatgtcaatgaatctgcaattgaagtttattgttaccagaggaacttgtcccaggcatgccaactgtaaatgctttgctgtgtttcagctggtagaattgtattaaactctcatggattgcctataaaaaattggtcttttttaaaatttaaaataatccttatttttcaacttgaattgtgaaacactaatgggaaatttctaaggaaaattttgaccacccttaggagaaataaatgcttaattactgggctttttccccttgttttgcttaggatttaattttggacaatatagtctactctgcctgattgaaacctgggatatacttcaaagagtatattgaagtgcatttgatggcctgtggaatttttggaagggccgtaggagcagactgcaagctgagcttccagaacactcccagcactcagaacttaagtccactacagtctgtaaatatgttgcggccagtgcagaaagctgttactgctgctgactggagggtcttgctgcccgcagtaccaactaagccaataaaatgcatgcccacagccagcctgtcttctcgagtatttccttcccatatcagatcttgcagtcacgcctaattggcagaaccgaagtcccatctacaaccctggataacttaaagggcgtctgataaatggagtttttagctttccagccatgatagtttagagggatatgctaggagaatattggaatggcaattgaaagagacaatcaacagtattctgtgcactcaatacacttaacttttcttatcttttttgtttttaatttccataggttattggggaacagggggtgtttggttacatgagtaagttctttagtggtgatttgtgagattttggtgcacccatcacccaagtagtatacactgtacccaatttgtcatcttttatccctcacccccctcaagttactctaaatcaagagcacctagtatgtagcccttcaatcaaatacaacgttgtatatcttttaagacatttttgttttgtttgttttctttaacaatataacactcatgaactatctaaaatattatatctcagactttatagagtacctaataatcacatagagggcttactaaaagatgtgctttcctgagatgcaccctcagaaatttggattccctctgacatgcaggaatctgtattttaattaaacaccccagatgattacactttcatctaaacataacacagctgactcccttggtttcagttggaacaccttcagtgaaagaaggaacattttaacagtttattccatcccactaaaaaggcagttggtatgtctcacacacattgcataattcgcaaagccagtttgtgacctttgccaatcaagcttgtcatctgatgtgacagagaaaacgatcaagattgtgaacgttctttgagctcatgtctctagctccactcattttgatgatatttggaagcagttaaagtagagtataaagatttagtttattctcatgtagtaaaagatccaggttggtgggtggcgggtcgtgaggggtggggggaactttgttgaaaattttctgagctgcttcctcagatttttatttatttatttatttatttaaaaggtcttgtgagactaaacttgtcagattttcagtagcagatgtgaaaatacatatgagcacattaattacatccatagtgggaaaaaaacacaaaaaggttctaggaaattcaggcattcagtctcagattgggcacttgggatcagatggcaactgaagcctatgctcatttcacatcaccaaatggtttggagttcaaaatattttagagattttaaataattacataatttttgatttacatgagatctcactttcagaagcaagtcacgtatttgaaagagtataaattatgatttatgtttctgtagctgtttttagagagctacagccctcagattcttgtacatcagggtgatgtacaaccctcagattcttgtacatcagggtagttcaattttaagagcagctagttggcataagagtaaacggtgcccgtgtccatggagtgctgaagatgcagatttcagaactttcatgaatttttaaaattatgtttccatactttggccggtgttgggggaggtctgctcgtcttatttctttgaaaatacacacttattgcagagagttggcggtagatatataaacctagctaacttaaagggcatctgagaaatggcgtttttagttttccagacgtgatagttgagaattaaactagtacccatcagttttctagactagctcagtctgttacacatcattatcctcacagaaattagaattagtcattagctcgcatctccttaaaattaagtggattatactgtgtaggggatttttttcccctctgctttgtataactgctaaagagctttatgaatagctaatgatctactttttaaaccattgttactaattagatagtctatactgctgtcacaaatgcttactctattatgtacatctttattaaaattagtaacagtaaaaacctgaacactcaggccctatattcttgcctgaggtaccagtcttttatcagggatttttacacagggtcaaaacttctgagctcacctggctctgttaaccccaggtgagttgttccctattcctaggccatttaaaaaagagaaatgctgagtttattattttagtcccactctttcagggctgtgtgttcccttctaccaagaccgaagtgaacttttctagccctttccatgatcactcactctcttctttcttttttggcaaactgtagtaatatacccttgaaattccctcatactcattcactcctgtagtccatccactgtgattgacttttagttccatcaactcaatggcaactgccctcattaaggtcagctccattaagtactctttagtaaacatcatattgacatcttttagtgcttgttttattcattgccctctccctttggtattttttttccattggctgctgtaccccacaatcaactgatttccctctcatgtatttggcttcacattctctcatctcctacacagatagttcatgatagaatggtatctatctatggtatggtagtattcactaaggtttttggtgataaactcccagactcctgctgcctcttctcttcctacatatctggatcagcagatagcaccactttttacacagtcacccaaactggaacctggaagtcatttgtgcctcttccccttttgaccctaccctcttttcattagtcaccaagatcttccgttctacttccatatcctgttgaacgtgttcatttctctccattctcactgccactgccctagttaaggcccttatcatctcagctttggattacttcagtgacctcctaatgggcctcatgcccccaggtttgttgccctctctcccccagtccatccttcattctgatgcctgataaaccttttaacaataaatcagatcttgacactcccttgcttaaaatcattccaggtctccccagtgcttttaaaatgaaatctaaatcaattagcaaggagggcctctgcatatctctctagccttgcctttttttttttttttgcttattctttgaaacttattttccaatggagtaaaaaaatcacagaattctaagaacatgccttgtttccacacttctgtgggcacctgctacttcctctctcaggggtcttctccctcactaactttgagttggctgcccccgatcacctttggagaagcttctccccccagaaactatcagcccctccctcaaatgcccctgtcccccaaatgcccccctccccaagtcaggtttagctgtacttggggcagggggcagttgagggaggggctgggccttttctctctgactcccaccactgtgctgtgttctctagtagcttgagcttcctgaggccagtgatcatagtctttctgtctttatcttatgtatttattagatactccatacatgtctgttaaatgaagaagtggcccctgggataatgttactagaagtctgaaagcacccaattttgtttttataagaagaaacaacttatcgctttattttttgggtttctcccctccctctagctatgtaaatttttgtttaagctatagctaagtgtaacttgatttacctgctcgactcatttattcaacctggtaataacttgacatgtaatttcaatttgaaactgattgagttaaagagacctatatgccttctgtaacatgaccctttaaaataaaatttagtagttcattaaatataaatgaagtcagttttattatctaactggcctagtatttttctttccagcatagatagtgtaggttttggtttatatccaatccagcaatttcctagcaaagtggccataaggaagttacctaatctctcccgaattgtgttttgctcacctgcagagtagggtaataatatctacctggttgatgggaggattacaggaaataatatctgcaaactgcttgtcctatgcacagccccttagaagtcaatatattttgagttatttccactttaagtagtatttacatgtagaacgaggactgattattaaagggtgaatttccccaaatttttctcatgcattgaataccttgaaatctttaaattcccgttacattaaattcccttattatattacattgtctgccttttcatattaagcaacttctttctagtttttttttgttataagagcatattcattacatattttaatgatcacccatattttaaaacatttttagcttacttcttcacaaaagggaagttagaagagtatctttacaagattttggcacccaccaatttggactccaagttggtctgagtttcaattagttactctggtaacatctgttttattgatagtagttaagtttcaaaaactaacaaatctactaaatgtttcagttgtccctttaagattacttaacagtgcttagagaagaattttagtctgtgtaagatgattaggtattttccttataatgtttaatggtaaatttttaagcaattaaaaagatctgaaaaaagaagtcatatagaactgaatggttattcaccaaatgaggatcctgttgttggtttacatagattatttttggaagatatggttcatatgagttaagaagacctgaactcaggaccaattaccatacctgtatcttggtaacttgctttcctgtctttgggtagttgagccagttggctctcttccaaaatacatttgggctacaagggaactgcctgcttcaatagctggtatgcaggattaaagagaagatgaatgttgtttttggcagcagctgctgttttgttcagggcagctctaataccatttcgtaggtctgtgcataaagaaaatgagctacacaaacacttaactctctttttgactttgtgacgtctgtaatatggaccctgacaatgcatattttaaaatcacttctcatattcatagagaaaaaaaggctttttttctgtattttatttttttgtttttccaagtttagaggattggtatcttctggtttcttgaacagcatatgtccccctgatattcttaggctgcatgcattttaatttctactgaaggaaaggaatctgcagtgtgacttatgtgttgcaacacacgttcagattaagtccatggtttgttttgggtgtttgtagctccttttcctggcatgtggccttttactgtaaggaaagctctgtgttcaggagggattggttattgagtgaatagtccactggagtctgctgatgtttgttctcaacaaaaatgaaaattaagcaagacctttgcctttgaggtcttcaacctcatcttcttgtaaatccctcccaccccagacttctttaaagtacttttctagtactgtctgctactgtctgctcccagaaaaggaccagccacatctgtcactgatgttaacataaattttagtcagtattacttccctctaccagaactgtttctactgcatgccttggttgtaacagtgcacacagatttctcattattttttttcctaatcatgtaaatgatttgaaggcaacatttagcaatgaaaggattattggaattttgtgtgttgtgggggaataaacgggggacacttttgatgcatgtttctgaaaacaaaaatcgagaactacccaggacgatatgtgtctatacaatgaaagactatccagaaatgaggggttatcacacaatgagcttgacctagagagaaaagttgtacaatgataaaacaaagtagccaaagaaaaatgtcatatacagctgcagagaatgttaacagaaaaaaaaaatagatgattacccaaactgaaaagatacattttagagcagaagtcacaaacacagggacattttgcttcagtgtatctccattagggtgtgtgtgagcgtgtgtgcacacccacatatttaggcaccctattccctcagagtttccagaaattggaagaagaggaagagaaaacaaagataaatattttgttgatatattggaacatgtcatttaattggaagtcagttgtctgttaactgaagctactctaaacacagttcagaactaagaaataaatgaaaaaggtgtgtgaactcaaagtcagtgtcaaaatattcatgtgctaaaggtcatactctttgtatatgggagatctactctataaagtcttttctcagaaagttttactcaaacatttttgaagtacttgtcttctggtttcctagccgatgcaaagcccataaaccacacaacaaataattgcataacacccatagtttgaatagtttctcatttgattcttactactagttcatgagatgggtaaggcaaaaattatttttactatcattttataggctgataaatggatactcagggagattagctgacttgtttgaagtcacatgggaagggactgagaggttttctggatccatgttctgcccatggtttccaaaagcgttttttttttttaatttcttcttgcccttggaaacctttgtttaaacagaatcttactcagaggcatgaacaaataaaacaacaaaaaaagctgagctgctctgcttaaaataaagggtgcgggccaataattctggcagaggcaattcactttcctcatttggaatagcttgaccatcactacactacacctttcattcattcattcattccaaaagtatttcaattgagctgtgttttatttttctattgctgctgtaataaattaccaaaaatttagtgactggccgggtgcagtggctcatacctgtaatctcagcattttgggaggctgaagtgggtggattacctgagctcaggagttcaagaccagcctggccaacatggtgaaactctgtctctaccaaaaatacaaaaaaaaaaagaaaagaaaaaggccagccatggtggcaggcacctataatcccagctacttgggaggctgaggcaggagaatcacttgaatctgggaggcggaggttgcagtgagccgagattgcaccactgcatcccagcctgagcaacagagagagactctgtcaaaaaaaaaaaaaaaaaattatcataattttgaagatcaaatgttcaaaataaggctcacttacatcaaagtatcagttggactatgttgctttccaaaggctctaggggagaatctatttctttctcttttgcagcttctagaagctgcctacatttcttgactcttggtcccttctctctattttcaatgccagcaaggacatgttgagtccttttcaggtcacatttccccgacccttcttccatcatcgcacctctctctgaacagagctggaaaatagtctccatttttaaggaccatggggattagattggacccagggaatctaggatagtctcttcatctctagttcttaacgtaatcatgtctggaaagtccattttgccatgtgtggtgatgtattcacaagttccaagaattaggacatggacatctttggggccattatacttcctgctgtaagctcccatatgggacacatgttgtgttcatcatcaagagtgcaggagactagaaataggcatgttagcagtaacaatatgttgcaagtaatgacctgacaatctgggggaaacaggaaaactaaggaacaaacaaatgaaacctaaaataaatttttaaaagttatacaaacaagtacagtaatttggaaccacttggagaagggaagctacaactacataactgagatactttgaaagtcttccttttatttgatgaacctgaaccgtcacagaaaagcaaagtttaatatttcaattcactgtttaagaaatcaaagtgtgtgtacaatattagaaagacttctgtgtagaaaagttaatactgaaaagtttcttttttatataaagaagtaagctctgcatttcagctctctcctgagtatactgatgaaggaggtgtcattactgcttggcaaaaatctcaatatttctaacttgattatctccctgtcctaatgtttagctatgccagaatgaattctttaattttatgaaaggtggatatttttaatggtagcaaagtatatgcaaaattaaattacaatatgaatttatttataatggctttaaaataaagggctttaagaaatataggtttttcagagatatttgaatgttcataagctaacatagatttaattttagcattgacccagtggatttttacaaaatgaacaaaactgtgtctttgtaagatatttgaatttaactgatgcaaaaaaaatttatgttgccctgatcaaatcattcttcaattattatagcagtctgtgttgatatattttaaggaactatcaaaggacatatattaaaaattatgatgacttgaataatatagactattcagctgtggcatggaatatggaacagacttgtctccatatgcttgatatttgcaaaagtattaaagttttagagatctgctatcgcatcacagttctcacaaaccatctaacagatttgcaggagttctgggacagtgtagaggggctaaaaggacaggagttagaattggacagacctgggcttactgatttcaccactcagaagtcaccaaacctccctgtacctcaactccttcatctttaaaattaggatatatgaaagttatttttttccttattagatatccaagatgcttggtgtatagtaaatgttcagtaacttttagctctattcatggaattgttcccaaaacacaaccagcatctgcattcacattccagcgctctgcctgatagcattgaactacctatactcctatttaaagccacacttcctacttgagaactgaatctcaccccttctttcctgctcaaggctgttgcccaagcagtcccctctcttctgacctatattgccaattgcttcctcctactggatcattcccataggcatacactcattctggtatttcaaccttcttacaaataaaactctcttcacccccaatttcttgccagctacttccccatcttggttttatttatttatgtgtttgtttatccagttgctctcctttgaaggaaaactcttcaaaacagctgttcatactcactgtttctggttaatctcatctcctgctttctaaaacccactgtagactggctttttcccataccacttcaataaaactgctcttgttaatattaccagtgaccttaatgttgccaaaccagtggtcagttatcagtccttatcttactcggcctataactggcatttgacatagtttgaccctattttccttaaagtacttttttaaagtcttggcttccttccatgatctttttgctttgctctcttggactcttttgtgggttctcactcttctgtcagatcccttaaggttagaacaccatagcatgcgcagttctttgttcttttttatacttttactcactcccttggtgatctcatccactcatttgcttatatatggaaggttatcatatttatatctgcaccccaagtttccctcctaagtccagatttaaatatcttgacctggcattatcactagaatatctcaaagacatcttaaacttaacatactcaaaatttaactcctgatctgcctcccaaaccttctctacccccagtcttccccatatcagttgatggcaacaccatctttccataccaaagttcttatagtcatctttgactctttctctcaaatcgtactctgtcaggaaattctcatggttcagccttcagaagatgtccagaatctaaccactttcagtcaatgtattactataaccctttcttgattggattacagcagcaacctcccaacttgtcggtttcattttctacctgttgtcaccctaaagtctatttttattatagcaatcagactgatcttatttaattaatttatttttattctgaagtatatatccctcctctgctcaaaatcttgcaatgaatcctattttattcaagtaagagtcaatgtctgggcaataggcttcaacgccctacatgagctgtcctctccattgtctctctgacatcatcatcttctactgctccctaatttactcttgctatgtcttaaataacagatgccttaaatagaccttaggaccttggcaccagcttttctctctgtatgcagtactcttcccacagatgtctgcgtggatcactgccttagctttgtccaatctttggtgaaattttgtcttcttaatgagacacttatcaatattaccctgttaaaaatttcaagctgtttccacccatactgcaatctctggaaagaaaagagtacagttagttctatggctcttctcgagactcagagatgcctggtgatttgaccctccctataagtcttctttcaacaaggactcaagaaacttttaggtgtacatctatacttcctccaagggaattcagctgaagttactaggtagcaggcactgatgtgtgctttattacaagcatatgtcatgcactgatatgtattaatattttattgttgagactggtactggtgggggtagctgctctagaaaatgaatagaagttcatgccatatccctaaaaagctgctcagctagcaaggtataagcagaaggaatgattctacctacccaacatcacctatcattgccacacgtatcttgagtgttcagtgactttctgatattagagatggcctgggagtctgctctgcttgtgctgtatgtactcttttgttatctaactttttttttttttggtgatgtagtctagcttttgtcgccaggctagagtgcagtggcgcaatcttggctcactgcaacctccgcctcacgggttcaaacgattctcctgcctcagcctcccgagtacctgggattacaggcatgcactgccacacccagctaatttttgtatttttagtagagatggggattcaccatgttggccaggctggtctcgatctcctgacctcgtgatccgcccacctcggcctcctaaagtgctgggattacaggcatgagccaccacgcccggccaacctaacctttcttaaaactcagcagtacacttggtgctgggcaaagccttggtgctgggcaaagcagtacacatggtgactcatgactgtgattgtcactctgaacccaagatcaccagtaaggcagagtagacattatgatcccaatcccctttaccttgctctaatcttatttttcccatagcttttgacaccttttgacatactgtataacctagttacttatgtttgttgattaagccttatgagggcaggaattttgttcattatataggcctagcacctaggacaatgcctggtatacaccagatactcaataaatacttgttagatgaatgagaaaacaaacgtttctttagtatttattcaatgccaggcgtaaaggattgcgaagaccagtaacagaatttctgccattacatttctttgagcaaaggatatgattatggtcatttctgagattggtccattacagagatccttagagaaagttcccaaatcacatcagaaagaggggtttagagaaatatatttccagggaggttacgtcttagcaatgacaatataaaagaaagctgactttaataaattatatttacacatgtgaaggaagactgttattaagggacagtttgtatgtaggggttgtctgtccaaaaagaagaatcctcaaacagaaataatttatacaacttagtcttcactgtggtccaagaaagatgatactatgaagagtgtgaccccacaatgctagaagtagccttaaataaaattatgcaggattactgctaaaatagaccatggtggtaagggaataaaaggatttcaatcttgaatacaaaccactatatccatgtaattagcattgtagttttagaaattttagcaaataataatcattatttgttctagtataatatctagttagcatgcagatattaattgaaaataataatccagttcctttttaatatggctagctatgtaaatagagaagatttctagccatgtgcatctggactcttgtgtaattttaagattttacagaagaatggtatctcataaaagaatgtgcacatattttaagatgtcttcacatctgcatggttttgtcatgctgtttatttttcctggaacgttcatcttttctttgtcagcccttgcaagtcctccttgtcctcctaaatccagccacagtcacattttctttctggcttttcttggcactgtcttcctgcaactggttctcccccttaaggttaattgattaattgcttcctcttctgtgttccgaaagtgtttatcacaagatgtgagtgttatttatttacctgcctctgagtttcaagggcatcttcaacatctttgttaccctagtgactggcgttgttcctgttgcataatatatattcaataaatggttattgaatttgagcatcaggatatatttttaccttgaccactgattgacccagcaaatatttattcaggactttttatatgcaagacactgtgttatgtgttgtgaagatactacaaagataaatcagttgcacaagttcttgaaactctacagtgtaataaggaaaaataagtcatgcataaaagcaactataatacataatagaaaatgttattttcaagccgatgtgtaggttatgtgtgttcgagagagagagagagaagacagattactttctgctagggttcaagaatgccttcctgttggctaaggaaatattttccttaagtggctaaaaagctgtgtttcaaaatattcttttgatgtctcacaaattcagtggaattctcttaggtctaaaaatatacatctctctcactttaacttggtgtgctattgtagattattggattaaagcactgctcagggattatgctgcttcttgccaagcagtctacatttaaagtagaaataagatgtttcttttggtgccataaggtatacattttatgcattctctagtttttagaagataccctaagggctaagtctttaacatgctgctacaagtttattcctaattgccattgggaaattggctgaagaaagtttttaacaaaagttaacaatattgtcattgagagaataattcaaaatggattttaactaaaagcttttaaaaactttggtgagcatagcttgaatgcgtaatatttaattgcatttaagccaataacatatattagactggtctttttgtgcatcaaggcattagatgttaaaagtttgaatgattacagatcttaactgatgatcaccaagcaatttttctgttttcatttagacttggattctcacttgcatttatcttcagctgctcctatttaatcctctcgtcaaaactgaagggatctgcaggaatcgtgtgactaataatgtaaaagacgtcactaaattggtaagtaaggaatgctttaccgtgctgtgtaaaaaagagctgtggctctttttcctgtgcttgttgataaaagatttagatttttcttgccccaaagtaatgttttcctaaagtggggaaagtaatcactgggttacaataaagggtttatagaaagcaggtagtgagatatttagggtcatggataatttgttggtaaaactggctagttgcacaccactgctgtgactgcttctttgctggtcttctccccatccttcataggcagtgaaggaccttggagagttcgctgtgtgctgatgggcttgccccagcttgttccccataatctctccagtgggtttcccagcatgttctattccccttcacatgtcttcctactcttctttaaaaagtctaacgaaaggaaatctgaaatggctattctcccaattcaatcagcaggaagaccctgtcacatgtcagtgggtgtttgctccttcagggaacatagagaggtgattcattgcccacatgttgaagggactcatctccctggtttgtcacattgaactcttccctcagcgaaagcatttgcattgcttccccaagtcactgccagctccatgctatgcttctccagatgctttctctcaagatcaagccacagagtgggcataacacatctggaatgcatccttcccaaccgtcacgatctaggggagatcttacatgcttttttgaaccatgtatgatttcgggatgaacaaagcttgagaagcaatttttaaattcctagttatttaaatactgactagagaacggaggtgatgcgagaataggtaagaaatgagggttgtgatatggagaaaaggatgaaaagtttattttttgaggtactgggataaggagtagaaggggctctatttatcttccctcctcttgatatatattttctggtgtatatgtcttattttgggtttataatagaacttcaaaacagaagtttaaggccactgttgaagtcttgctagcttagcctcaatatagaattagaaacacacagacacaatttatacaaattccttaggacccagagagttcaggtttaaaagcattcttccatgcagcagcagcagatgtatcagtatgtcttacttgagggaactgtgagcaaagccatgcaagagttgtctgctatatacccagttagctattgtgtatagggatgggggtgaggggggagtacttttttttttttaaaaacatactgtttaaaaatagatcatgttttttttccttgagttgcttgttagtttgtgacccaagaaacaatactaaaaggacagtatgtgtttaaaatctaacttttgtgaatgacctggattctagtctagacttaacatgtgatggctgtgtgaccttagtcaaggcacttgaccccttctagcctcagtttcttcatctgttaaatagagataatatgtcagcgggatctgaggataaattgtaataatgcagcctaagcccatagtgagcacagtgcctagcacatagtaaatatttgacacacagcacatagtaaacagtgttagacactgttactattttatgattatgtgaacttagggaagaggaaccatactaagtgcctcagttcagagctgaaatgagtgtcaggtggcattactggaagtaggatggtagtaagcaccatgagatcagtcactatcttagctcccatctgactacttacgcccatgcctaaattcatttatcaataaattgttctcagattctagaagctattgacaagaccaagcaggatcatacagaaaatgttatagtaaacttgtgcgagatgacctagtctagcaaacatgcacatgatcttagctttctataaggaagataaagtttttagagacaagatgaaggaaaaaaataagtctacaaagcaagatagacccttgaagatgtaatggtgcaaaactactttttaatgtatgttatcttaatacccaatggtctaagtcagttgttggctaaatttttcatctgggaattttaatggtgatatgagaagggcggggaaagcaaagtagatgatgttcattaaatgtttgctgggtgagtgaagaagaggaaatgatgtgctttactttagtgagcatcacaatcacctgtcatgctgatgaaaattatgggtacctctattcacaatagcaaagacttggaaccaacccaaatgtccatcaataatagaagactggataaagaaaatttcacaaatatacaccatggaatactatgcagccataaaaaaagatgagttcatgtcctttgaagggacatggatgaagctggaaacaattattctcagcaaaatatcacaagaacagaaaaccaaacaccgcatgttctcactcataagtgggagttgaacaatgagaacacaaggacacagggaggggaacattacacactggggcctgttggggggtgggggcctggaagagggatagcgtttggagaaatacctaatgtaaatgacgagctgatgggtgcagcaaagcaacatgatacatgtatacctatgtaacaaacctgcacgttgtgcacatgtaccctagaacttaaagtataataataataataaaaattacaggtgcctgggactacaccaagaaactactttggtaagtctgaaaaagggatccaaacatctatatttttaacaaaattctgaaaggaggcttaagagcaattgctctagatggtctcttctgtcattgtttagaaaccaaaatgtcttgactcttatttaatctcaattcctattatgactagttatcatctgtgttgtatagacttataaggatgccagattaatacaaatctccaaaatatgtttaaagtaggtcactactttagaaccagcagatcacagtctctcagggacttatttaaggtacagctctaagggcatcactataccgaatctcaacttgtggagatggggtgtgagaaccctaatgtttccaactagcatctacataaaagttatttatgtagaagtatcaggactgcttaggccataatgtttatagagtgtgtgtgtgtgtgtgcatgtgtgtgtgtgtattttaatgtttgaaaagaagtggagtgttcctatcctaacttactaatttcatttattcaaacagcaacatgagtgactgttgagatttatggatgtagataatcacagcctttctctatacatggtggggcagagcatcttgaaggaagtcgggaatactaggtttttttaataccagctccactattacctggctgtgtggccttggacaaatcactgaatctttctggcattcaatttcctaatttttcaaatgaggaacatggacatgatcaatagttattaatctagggctgacaaacttcaaatgcttacacatgaagctcagaattgtgctgccttcatatgccttatgttcatttctgaggagagtgtgtagcttttatcatttctcagagaaatctaagaccccaaagaagataagaactactaaattagagaaacttggaggcctttttcagctttgaaattattttattccataatgatgtttttttcaggttgcacctcttggaataattcaataataactcgactgaaggaggttatataggatgagaggaatttgctttgcagacataaccttgctacattttatgtgagggaaagaaagtggcctgacagtccatttttgcctgtgctcacagttattccttcaatggaaatcattcctttgcagcccctaccagtttcatctttgcattaggtggcaacctgcactgtgcccacacacttgaagtttttttgcttgaagtgattctcatgtcacagaagcccatgatttatgactttaatgtcaaattttttttttaatttcttcttgaacactttgtactgcccccacggtccccgtggtttttattcgaaatgcttctggcctgtttactaagcataatagttcatctatttacagaacattttgctttgtttctaaaaaaatgttgcaacatttgttttaaatggcaaactcagacttacctaaagaaagggtcaagtatctgggaagaggatgaatcaagcttttataacttgtatctatgggatagttaggttgcagattttagtagttgtaattgttcatataggaaggaaatccagatattgaaaaagttcaaactagttttctctgtgtattctccctactcttctctttgtttgtgacgactttgtttcttcatttgaggcaatttaactcaatatgattttgttgctaaaaagaaaaaatgttttgtctttaactttctattgggtaatgacttgtcttcctcaaaatgtttcttaaggtcccatctccatctcgtaaaggtgaaattactctgtcttttcaaggtcagtctacaaattagtaagatagcattagaacaaaggaatctattgtcttcctggtcttgtgataggcacttatttcaagttggctatatttatacatgattcatggagattatcgtaatgagagattataaaaatatcctcagataacacattttcacactttgaatagacagttttgattaattctgattatttttcttcttcaaatattattgttctcaagtagaattaccactgcattaaagtgaagaattggagtctctgaatgtccccatttctggatgaattctaagcttttcttcagatgacctaggtatatcattaatagtgaagatttcacataattttgtttgaccttcgaaaaataatagtttgaaagatagttttcaaattttattttattaggttgcaatatttagcattttaatttctgctcctgtggtcagcagaaaattctttctctatttaaagctaaatttaggacattttgtattttaaaaaaaggactattagaaataatatttattaaaactctagatagattgctatattatcaagcatatgagtcttattgttaggaaaacattgttcttcctttatagaataaactacttggataatatgtcttctgctcattgaatttttatttttatcttttgtagagatgtgggtcccattatgttgcccaggctggtctcaaactcctggccccaagagatcctcccacctctgcctctcaaaattctgggattacaggtgtaagccaccatgtgcagcctgctccttggttttttagaacaatagttatgagtacacagacaccatgcagaaacactttgcatggattgtctcctttacttttcataaccttacaagataactatttttagcccagtatattggttagaaagctgagatctagtggtatttagtaatctgattatcatcacttgggaaatagttgagtagaggtttgaccccttaactacctatcattgaggcacagtaatgcagttcataatttataatgccttcccagtagtttaagatgtaagtttcaactacaattaatgaattatcattgtaacctactgaattttatttcctgaatagtagtcaccttatcttactgtgattaggaagctatcatattgtagaagctcagttagttacttaattggaatcatcatcataaggtgtgtcatcatcataaggtatgtcatcatcatgaggtattatacactccctaaatattgtagtttataatctcttctatttgaaagaaaaaattgttatctatctttatttagatattctttactgaggaacgatgaccttgaaaagcctcctgaagttgtcatcttaatttttttttgagacagggtgtctatcacccagtctggggtacagtggtgcaatcatggctcactggagcctcaaactcctgggctcaagtgatcctcctgcctcagcctctctagtagctgggactccagagtagttgccaccatacctgactaattttttaaatttttttcttgagatggactctcactttgttgccaagtctggtctcaaactcctggctttaagcaatcctctcaccttggcttcttaaagcagtaggattacaggtatgaaccactgtgccctgccaatttatttttaatcactaattgagttaccattttaaaaaagtccagttacatttgaaaactataggtaatacacctggttggttagatttttagtcaatattagtggtagataattttttaggagaaacaaagaagcaaaaagcttttttggggggtgttatttctgaaggttgaacaactaacatagtaagctactgtacctgagtgaacgatgatagaagtgtagagcttttttcctctacagccatgattattaaactttgctaggtctgaaaatcatagactctttccccagaaaagttgacataggcacagcattttgtaaaagtgtcaagggagtcatagataactaatgaattttgattcctgccttggacttgggagagacagcataacaagtaagggtcttgcaaagatgtccaaggtggccactgatctcatgagatttagggacctcagtgacccttgaaccatgagatccaaaggtttttagatcagtgatctagagaggaagttaagtgttctgactttgagttatttttcttctgagtccaaatacctgcagggtgtggtatgcagccagtgccagtttggcttgcaaagacttcttgttggccaagtctccagcatttccaaatgctgttctcccatggatgtttttccagtgtcactggacatggtcaatcattaagacatccaaagaagacagtaaatggttcccacctagcacagttagctcatcatccagtaacttgtttttgcagttttttattttacaatctggcatagtattgacatccttgcactccaaaccagagctcattagaatgagttggcctttggtaaaattttccgtcaattaatttttttttgccatgaatttctttagtagaacagatttgttttttaaaatacgtagccttggttacttatccacttaaccctaccaatagggaatactccctggactctagacctggttcctgcttttctagtttccagtttgcatgtaaacatttaatgtatattcaaattcctagtggtcttctaatcttttgtctgtctctgttttctctttgctccaaactgtcttcccatactaatcttggtattttactaccaggttccttctctgctgaaacacattcagtgttgcatcaccacttacgtaagtaagtcaaaccagactgaatttatagaaattatctagtttgccctctttatcttacagacaaggagtagaacttccacagagatgatgctatgtgtctaagcagaagcaacttgctgtggagttctgatggtagaactgttcctactgtaccttactacctaccatttgacctccctagctcaacatttggagggtctctctcaaaggcagttttcctggaatatttcttatctcttaactggtctccctaacacattatgagctttcccatttctttgcctttgcataagacccttcctctatctagactgtcccactccctcgcatttagtattaatagaaattcatactcattcacataccatgcctaactaagcacttagtattttatatgagttattttatccttataaatccatcagctagattctatatgattcccatgtgacacatgtaaaaatttaggctgaagtagatgaagtgacttgcccaagacttctgagttagtaggtttcagggccaggatccaacctgagtggtctgtctccagaacctgtgctattaatccatacattaaattcactgcctctaaggagtattttccaagttgcaggccataggctccctctactctgtgaaactttccacagtgatatatatcagagaagcagaatttatttcatggttatctgtgctttctggaaggtgagtcaatatactacggggttgaatatttcctacccaaaatgcttgggaccaaaagtgtttcagatttcatttttttttttcagattttagaatatttgtggttttttctttttaatatactttcaactattatttttgattcagcagctacatttgaaggtttattacttgggtatattgtatgatgctgaggtttggggtatgattgatcccatcacccaggtagtgaacatggtacccaataggtagtttttttaaccctgtcttcctgcttcctatctagttgtccctggtatctattgttgccatctttatttccatgagtatgcagtgtttagtccccacttgtaagtgagaacatgcaatatttggttttctgttcctgtgttaattcactgacaatgatggcctccagctgcatccatattcctgcgaaagacatgattttattgtttttttttgtggctgcatagtattccatggtgtatatgtatcacattgtctttattcagtccattgttgataggcacataggtcgattccatgtctttctattgcgaatagtactgtgatgaacgtacaaatgcatgggtttatttggtagaaggacttattttggatatacacccagcaatgggattgctgggttgaatggtagttctgaattctttgagaaatccccaaatttctttccacagtggctgaacagtgtatatgcattcccttttctctgcagccacaccagcatctgttgttttttgacatttttttaaattatactttaagttctggggtacaagtgcagaacgtgcagtttgttacataggtatacacgtgccatggtggtttgctgcagccatcaacccctcacctacattaggtatttctcctagtgctatccttcgcctagccccccaatacccaacaggccctggtgtgtgatgtttccctccctgtgtccatgtgttctcattgttcaactcccacttatgagtgagaacatgtggtgtttggttttctgttgttgtgttagtttactgagaatgattgtttccagcttcatccatgtctctgcaaaggacatgaactcatcctttttatggctgcatattattccatgctgtatatgtgccacattttctttatccagtctatcactgatggacatttgggttggttccaagtctttgctattgtgaatagtgccgcaataaacatacgtgtgaatgtgtctttatagtagaatgatttataatcctttgtgtatatacccagtaatgggatcgctgggtcaaatggtatttctggttctagatccttgaggaatcgctacactatcttccacaatggttgaactaatttacactcccaccaacatgacagccattctaacccctatgagatggtgtcttattgtggttttgatttgcatttctctgttgattagtgaggttaagcaatttttcatatgtttcttggccccttgcatatcttcttttgagaaatgtctgtgtcttttgcttacttgttgtttatggggttattttttgcttgttgaattgtttaagttccttatagattctggatattagatctttgttggatgcatagtttacaaatattttcccccattctgtaggttgtctatttactctgttagtagtttcttttgctgtgtagtagctctttagtttaattagctcccaattgtcattttttgtttctgttggaattgcttttgaggacttagtcataaattattccccaaggccaatgtccagaatggtgtttactaagttttcttccagcatttttacagtgtggggccttacattttaatctttaattccttttgagttaatttttgtatgtgttgaatagtaggggtctagttccattcttctgcatatagctagccagttatcccagcaccatttattgaatagggagtcctttccctattgtgtatgtttgttgactctgtcaaagatgacatggcttgtatgcatgtagctttattttagatttcagattagagatgttcaatctgtataaatgcttagaaagtaatattaacacaataatttattaagcgaccacaaatatctacaacttgactttctaagttctcttccaaatgtaattagaaggaaaccaatgaaaaccaaggacgtaataatgggatactgcgctataggtagatgtgctgagccccagcacacaaagccagggtttgcatcctacctgcaccagttactcctaatgcgtgtaccttggggataccggttaacctctctgagtttccatgacctcatttgtaaaataagcttaatataatctctcatggggttgtggtagagactgtatatgaaaacttgttttaaattataaagcacgtttcaaaaacagtattatttttcagtctcaccagaaattgaatatcagtaaatcagcttaacgaataaattactttattattatacactgccctaccaccagtggtagaatgtagatttcagtgtttttatcagggaacttcctataagccttctttgaaccaatatttgtatataaattattcatgaaatggaattggtcaccatactctttacattttctttgctttttagacatatactcagatcccacactttctaaatactaatctagttgattgtatttttgtatttttaaaataatataaattatgcttaagaaagggctggattgttaagcagcttgtgttcagaatacctgccacttcttgctgcgtttttctatctggaatgcctttaatttacctggtttattcaaaatgagatagaatattgctgcttacttatcgctttcacatctgcaacaggtctgctctgtctttgtcactagggtttacataaaatgactagacgctttttcctttcaaagatgaggatgaagattaagaagagaaaagaaattatggaagcttggtggaaagtttgagaggaagaaagttaagtgtaaagaataagggagttaacgccaaaaataaatgaacaaaaatgccgtcatgtctggcagctcaggtgtgttttcaataagcagcaatggaatggacacggtggctcatgactgtaatcccagcattttgggaggccaaggcaggcagatcacttgaggccaggagtttgagaccagactggccaacatagcaaaaccccgtctctgctaaaaatacaaaaattagccaggtgtagtggtgcgtgcctgtaatcccagaggctgacacatgcgaggattccttaagcccaggaggtggaggttggagtgagccaggattgtgctactgcactccagcctgggtgacagagtgagactctgtaccaaaaaaagaaaaaaaaagcagcaatgattctaatattttatttagggtggtatgaagaaggttgatggtgaagattcacaagtggaaaatacccagaacaagtgcccagagggtgtagagtgtgaaaatatgtagtggagctaagagacaagggaggctcttctctccagggacctgagttctagtctagacttaacatgtgatggctgtgtgaccttagtcaaggcacttgaccccttctagcctcagtttcttcatctgttaaatagaggtaatatgtcagcgggatctgaggataaattgtaataatgcagcccaagcccatagtgagcacagtgcctagcacatagtaaatatttgatacgtgttagacactgttactattttatgattatgtgaacttagggaagaggaaccatactaagtgcctcagttcagagctgaaatgagtgtcaggtggcattactggaagtaggatggtagtaagcaccatgagatcagttactatcttagctcccatgtgactacttatgcccatgcctaaattcatttatcaataaattgttctcagattctagaagctattgacaagaccaagcagaatcatacagaaaatgttatggtaaacttgtgcgagatgacctagtctagcaaacatgcacatgatcttagctttctataaggaagataaagtttttagagacaaggtgaaggaaaaaataagtctacaaagcaagatagacccttgaagatgtaatggtgcaaaactactttttaatatatgttatcttaatacccaatggtctaagtcagttgttggctaaattattcatctgggaattttaatggtgacataagagaaggttgcggacagcaaagttggtacagtgtgtaggaaggaagagaacctagaactctgtaacttactagggttatatgcctccaaagtcagttgccggtatagctctcagtaaagaacatggattgagtgtttataatgtgtttggcacattcatctgtcttaattctaaatgaccatttgacataggtatcttgatcttcattgtataaatgagaggaaccaacatacagagtttagatttgtcccagattcctttgtcctaaacctattgcttattcacttatgccagaatgactgatgtaagtaagtcagacgttatttaaagccttcaaaagaacctaggcaaatatttgagacttctcaaatgttcagccgaaggtaagagaattaagttacatcacaaaatgaatctttctaaattatttcttataaagcatttgcatctcataagtagcattgtttttttgaagtttcactgatcattttttaggttcaacataaaatagttctcaaatttgggtgcacatcggaatcacctggaggacttgttaagtaagattcagtagctctggtatggcctgagaatatgcatttctgacagattccctattgctggcacatcactttgaggtccacattttgagaaccactgacataaaaagcatgtattttatgattaatcctatggaggaacaagtaggaaaatagtccttgaggggaattccaagatcacaggtggaagctgaaattcagatcatgtttccaaaactcagtaggttatacctagccaggcataactgaatttggagtctaaaagatctgtattatcacttttttattttgaaggatgccttttgattacagagggaaatcaaggattaaaaatcaatatacatgtaaatattgaaattcattggtaactttaaaaagcacaacagttttgtgtgcttttctccaaagcactacaaatatgattaattgatgtataagaattttcttatggaatttttttttttgtctctgtaggtggcaaatcttccaaaagactacatgataaccctcaaatatgtccccgggatggatgttttggtatgtaaactacatttctgagtttcattttagtagctcatagaagaaatgggatcattcatattgagatagtacactagctgctatttaggagcttgcttattgtcaggatttgaagaatttatctttggaatttgacttgcaggcttttttttccccctcttaagcaactgttaaactttatcagtgatcatttcagcacatatatcctagacctgtatagttgttcttcactatccttaggaaattggttccaggacaccttccgaccctccatcctccccactcactccaccatcaggatacccaaatcctaggatgctcaagtcatttctatgaatggcatagtatttgcatataacctatgcacattcttctgtatactttatttttaaatttttatttttcaccatggaaggcacactggaactcctgtgtgttccaggagtttagttccaggaatttggaactaaattaaattatttctaaattaattctaaattaattctaatactgaatataatgcctatattacataaatcattgttatgctctgcttttaaaatttgctttatgttttcttattgtttgaatattttccatctttggttggttgaatccgtggatacagagggccatctgtacttccaaagtaaatacgattatcttctttagacctagccacagattttctcattttccttaagcatccccctaaaaaaggacttcagtgtaagtttatgtaaattctttttcatagctgacctacctcacttgtttaatttgaaagcacaatttgatttactagcagtgcaatagattcttagaaccttacaacaagtgtatgtgtttactattataagtactactctgttgctgaaaccaagaaattttaattatttttctcattacacagtatcagaattcatacattaagaaatatttcaagcatagagaataatgtaatatctgtatacctatcacagagcttatcaaaccttaccattgtgcaatatttgcttcaaatatatttttaataatgaaacattacaaatgtaactaatgccctatgtatgctccttctctgtcttcattaatccccagtttgtctcttagtggtaacacttattctgagctggactttttcattccaatgtgtgctttcagggcttgtaatgtggacagcattcactagtgttatgtagagcaggaggagcattctcttatatgtttactgcagtgtatgcacccatacataagacatggaactttttgaatatttaataattttatgttaagcttctatactttgaagttcgcttatatctttcaacatgctttgacaagttgttttatgttgactcatttcaactactgtactgtgtttcttgagtaaatttaaaaccatttgagttactctcttctctacaactctcatctttccataatctaccaaaacttattaaacatctactttgtgtaggttttgaccaatttttgtggatcagtccatcaaagttgttaccaatttaaacacatcattatgtgcttgtgcttaaaatgcatttcaaagactacaaggctgtcatttcaagagaatgtcaaagctacatttctttttattagtaagcattttagcatgaactacctaaagttttttttagaattccttagaacatgtatcttcatgttgaaccatttatcctcaagcctatgtcaatgtctatcctattgagttagggggagttctctggaaatagaaatggaaaattattagcagcctgaaagttaattaattttattatttggttactaagcaagtatttagttaggaaaaactaaccaggagccagcactttggcagacacttgaagaaacaaacacacaccaattaggcaatgatctttagttcaaaaagtttagtaattgggaaggcaggcaggggggattatgtggcctttttataacacttttccatttgcaaaatgatttcacatattaattttcttggctgattagaaaaactctgaggaaggtaaaatagctattatgattcctattttacaaatgatatattgagctttacagcgcaaaagtattttgcccaaggctatacaattagaacgtgacagagtcttaaaaagaaacccgtaggtcatcagactcttaaccctgggttacggaaagtgaggatctatataattacaacacttagggaaagtgtttgaagggaagcctctaggtaataaagacccaccccagatccttcctgactgcagcagcatttaggattgtgattgccaagcacccaaagataacctgctgctctttgaaaaacaaatgctaaactgaaaacaactaaatagaattttttgtgctggtataatcatagaatatgctaaatcatagatttcaatgtcggcttgtgatagcaagagccaagcttgtgaaaataaagctgattcactgccacatattttgtgcttgtttctgtaattgagaaaaatctactattgattctggcatgctatactggcacaactggagaaacactgttcttcttggccaccaccatgggcaagaaaaacaaggccacgtttagagcattggacagaaccaggggtctcaccctttgttttaccttcattttgacatgttcgtttctgcgaagaccagaggagtttcccagtggggaggcctctgattatttgcgagtggtcagagtcccagctgccttatcttttgtctttaaacaatagagggtggagtgggaatgggtgccaaaaagattctctggtgccaaagcaataaaattctgcccatgggcactgggcagaagaaagctctagaatagaatacacttttatttttatttttattttgccttcattttaaaaaacagctaaattctcctagaaaagggagttcacttttccaaggtccttcttagaaaattacctagcagattctgtctcacaaatagcagggtgtgatcaaccacttagtatttaaatctaacgataatcaccaatgctattttaaactctttgaaactattatattgccggactgttgaaatttcctttaacttcctaaccttaaagaatacctggatatttctggtcttttacatttactggtaatgttattgtagaaagaataaaaatggcattgctgaattgttgccacacaccttcattcacttgaacttccatagcatttacataaactgtgggttgcttgtaatatttgagtttaaaaatcttcatttttaaaaaaatttttttatttttagctattatggatacataagagttgtgtatatttatggggtacacgtgatattttgatgcaaccatacagtgtgtaatgatcaaatcagagtaactggaatattcatcagctcaactttacaatttctttgtgttaggaacattccaattccactcttttagttattttgaaacagacatttttattaactgtgccacctattttgctactgaacactggatcttattcgtttttcaatttttacttaaagtatatattcatcagttttcttttagttttttatttgcaaataattttaaatttacaaaaacgttgcaagaatatttaaagaacacccatgtgccgtttaatccagattcacctatttcaagtatttttaacattctgcctcatttgctttatcagttgctttctttctatgtatgcatgtgtgttttattatgtttctgaacttcttgaataagttgtatactgatgcatacatggccatttactcttaaatacttcagtgtgtatttcctatgtgaaggatattttcttacctaaacacagtacatttgcaacttcaacacattttacaataattatagtactttaatctaggtctgcatttcaatttagcttttttgtttttgtttttgttttttgttttttgcatttcttttccttcagtataagatcctatctaggattggaattttcattttgataccatgtaaataatgactcccaaagatgccttgttttgggaatatattacctgacatggcaaaaaggactttgcagatgtaattaaattaaggatttatagatggaaaagttattttggattatccaggtggtcccactctgatttacatgagttcttataaaagggaggcaggagtgtcagagccagagagggagatttgaaaatattacactgctggcttcaaagatggaagagggattcacaagccaaggaacgcacatggcctctgaaagctggaaaaggcaaagaaaatgatttttcctttacagcatccaaaagaaacacagccctaatgaataccttggtattagcctattcagacctattttaaacttctgacctccagaactgtaaaataacaaatttgtgctgctttaagccaccaagtttgtagtactttgttatagcagcaataataagctactacatcctgtctctctagtctcctttaagatttcctcaggctttctttgtctttatggcactgacatttttgaatgagacagtcctcccctttaagaaaagtgtttctctttgcaacatttgattgatgtttcttaatgcttctgtggagcttatgcaattcaagtgacaatgctacgaaagtgatgatttgtgctcaggtaaccacatccagaggcacacagagtccatcctctcctcatggtgctgtttgttttgatcatctggttaagatgttcaactgtttatgtatatttactcattcacaatttggggagagacactttaaggccatataagcaatccttctccttctcaaagtttctcccacagatttagcatctattgatgatacttacctgaaccaatctttgctatcattgtcagtttttcgttcagactttttaaaggcacagctaaatatggaagtgattggcagcatgtgatgccactctaacaataagccttccaaacttcttccctcttcagtacccttcctcaggtatgcacaatttcacttcttggctattctcatctttttttgcctggccctgttacttacctctgggctctttccattttctttgatacctgaggatttattatttgatgttttgattcttcctgaatagacagagtgatttaaaaagaaatacatccaatcatttcattcctgtttctttatattcattgaatggctttttgctgctcttaggaaacaggaaaaggagctgcttcaacaaggcctccaagactgtagtgatctagctgggcacagtggctcacgcctgtaatctcagcattttgggaggctgagacaggtggatcatgaggtcaggagatcgagaccatcctggttaacacagtgaaaccccgtctatactaaaaatacagaaaattagctgggcgtggtggcgggcgcctgtagtcccagctactcaggaggctgagacaggagaatggcgtgaacctgggaggtggagttttcagtgagccaagattgcaccactgcactccaggctgggtgacagagcgagactccgtctcaaaaaaaaaaaaaaaaaaaaaaagactatactgatctggcacctttccacatcctttttcagtcctttatcgttatcgttaccagttccttgctgccacatagcttcttgcctggctttctctattcctggaatcctgattggttcttttgtctagctaacacctgattcttttagattttggttttgatttttgatgaaaccaaattgttctactggaaactcttctcatcgcactcctcagagtagcagttttggatgtatttgtgggatgatttaatagataatcttccaccaacctgtaaacttcacaaaggtaggaactatattctttttgctctgcattagattctcagtgcctcatggatgctcatatgcatttaatgaatatttgctgaaagaaagtgaagaaaggaaggaggaagaaaatttccctaccatataatcatttgtaatttccttgaggcacagatttgaacttagttcacaaggttataatgatgaggtgagtgcctataactcgtcacacagtatctgcactgcaacgttattatcttcttttagatacgtgtgaattttttgaaatattttaggtgtaagaattgtattatgcaattttggggaatcacagcttctttaagcctttggttctactgctttaattgagttatttttgcattcaagaacctgcttattgttcccatttactaaaggctgagagccagattcctgaacctaatgttcatgactctctgtaatccttccctaccctaaccaactcatctgaatacccattattccccaacacaaacttgccacctcagccacatgcatcactgcctcctaaaataatgcgcctgttctcaaaggatgccctttcttctcccatccacttatccaaattctacccatagaatcatgcaatatcttggggcttaacccatcttagtggtcatctcctccaaggtcagtttaagatttctctttcacacaaggacattttctgctctttcagcccacattttctctcccatgtatcatttgatcaggtgctactctgtgctgagtgttttgcttgatgctggagcttggtggtcagcaatactatcattgcacctgctatgtggaccctatcttttagtttattatcttgtgacttctatggaatttgtccagaagctattcatcttgggtacatgatcatatattgcttacatagagcttaatattttaagtgttatatatttgtctcaaagtattatcgtgagaaataattaaaatacaaaaaatatgagttgtggtgtgcatataataacatcagataccatcattattattttgttgttgtgttctctttttactatattttgactatctacagagcaaggttgaaatcttttactcctgtgtatctcctcaccattctttagacaatccaatgcatatagtcgctcctcaaaaaatagttatgaataaacatttaactcttggcaaaaatgctagatttccaaatcttgaaggagcccatcttattagtttagattcctgtggttttcactaatagtaaaagtcccattattataaggacactaaagaatagcatgtcacctaagggcatgggtgcaacaggccccatgaaaagactggaaccaggattggaaactatcagtttttccttctccatctctgaatctctgtttctccgcctcattctatttgtgcagatattttctcctttgttccagtcacttggtagaatgtaactgcccaagagctgccaggtttacatatttcaatttcagccctattcagagattaattatcagtttctaatgatcaataccaattttggggaggaacgttctggttggcctatccaagtcaggtgtctaccatcatccaaccaactatggctacagggaaagttcaccgtttgtaagcatggattctgaggctccactcctatgatgcggcattctctctataaaaatagacgtcagccaaatagacacttgtagtcaactatttatctgcctcgatctcccatgtaataagaaagtatttatttaaaaggaataagcataaaatgttagttgaatgaataaaatatgacattaaatgctattttaagtagttcttttgtatactgtactacagcacaggggcccgtcttcctaataagtgattatattaagatgctgttcagattatctaggccattattctctgatagaaatacaatggaagccacgttcataatcttaattgtctggtagcaacactaaaaaaaaaataaataaaaacgagcagctgaaattaagtataatgaaatttatttagccccatgtatccaaaatattgtcatttcaccttaaaatcaatataaaaattattaatggaatgttttatatgtttcatattattcaaaatccacagtgtgttttacatctgcaacatattacagttcatacactaaattttcattagaaatagttattccatatttagattttttaataaaatttaccattgagaagatagtcacatttctgtgttttccaaacatacttaaaagttccagcctctaaatagggtgatattattgacttttcaatttaaagaaattataattaagtaaaatgaaaattttagctctgcagtcacattcaatgcattttgagtgagaggagctgcagatggatagtgggattcccatattggccagcatcgattagaatttattttatagatggagaaactgaagccctgaatagattaaatgacttgcctgaaatcacaaagtgaattgacagagttgggactaaaaactgggcctctagttcatcgttgctgccctcactccccaaacccctgtgctgttccttcactgttctattttagggcgggcctaggggccatttataggtctgccctgggaaataacacatatttagagtgctttggttgattaccatcttaatcagattgagatttctacggcactttaattcaaacaagaaatgtaaagttagatgcctaaagtgaacactgaagacccaaattaattgtttagggagaggactaaaataaatcattacaaaacccagctaatccaaagagaagttaagatttcggagactacactggcaacatgaacatgcaatattttgcaaagtgcttttataggaaacttttaaatttgcctgcccatggaatgtttgagagtagaatgtcagctttctagccacaacgaaaaaaaccaaaagaacagtaaataaagtccacaaatggcaattgtcacaattcctcagagaaatttaccttcattggtatgactctggattaactgaaatgaaacagaaagaaccatttctattccaaagagatttcttaattgagccgaagaatcgtgtctggtccagacgtgagaccacatgggtcatagtgaattcagcagaaaaaaacacttcagttttatgtgctttcactattctcatggctactagaacaaaatgagtgatcgagagtcctggtttctcgcgtttctgtgtaaatttgttatgtaacctggaacgagacacttcactactctaagccttgattttcccgcttatacaattggtgagactggtcctcagtattccttctaggtctggaattctggtgattctgatgacatggctctagattttgattgcacatcccagaggtagatgctgaaagcacaaacagaaaggtacacttcgtgccctaggcatgcatacgtctaattccagcaagactgaagtttgacacgaattaaagaaagtaaaacaaaaacaaactgggtggtaatgctggtatttagattaatcagattagaaggctgggtgaatactccttatgaatattcttagaaacctcatagtttatgagccaggcttttcactctgagaatctctaattggtatttcaccaactcctgtgaaaagaacgttctcactgggtagatactagctttaggccctataagaaagaacatagatatctctctgtgaatagctaccaaattgaaagtaatgtaaagtgcttcagtgaagaagtgttacagtattattttgtctactttcagcttcttaatccgtaaatcttaccgtgactgctcttaaagactgtctgtggggtgggcgcggtggctcactcctgtaatcccagcactttgggaaaccgagatgggtggatcacctggagtcaggacttcaagattagcctagccaacatggtgaaatcccatctctactaaaaaaacaaaagttagctgggcgtggtggcagatgcccgtaatcccaactactcaggaggctgaagcaggagaatcgcttgaacccaggaggcagaggttgcagtgagctgagaccacgccattgcactccagcgtgggcgagaagagcaagactgtctcaaaaaaaaaaaaaaaaaaaagactgtgcatctaataccaatgtggaataatgtgtcaaattatttcacctaataaaccagcacagtttcttgaatgaggcaggagatagagaggagaaagcatgccagaattagcaagtctccaggatgattttaaaacacatggtttatttcagccggtttctcaaatttactggttctggaactacagagattctgtgtcacatattctgtggaattaatatattaccttttaaaaatttatgcttagtcttagaatgtgcatatatttctgttgttggtctttggaaacagtcatattcctagttatgtttagaatacttttaattttattcattttaaattatatattttatttaatcatttagcgagatcatgatttggtgttcatggtactttaaaattcctaaggaagttcacagtttttgactgaattgtcagtggtagtgagatgctagcagtccaatatcattgagtcaatatcattgagtcagtttggattctagaataataacatgttataagtacatagggatagaaagcacatgtttttagtagtcctagctagagctgctataattaaccaagaaaacataagacagaagctatcaagaaagaaggtaagaaattgagagcatggagtcaagaaaaaagtttttacactcaaagcagaatctatatgtttcttcttttggaaacaatacatacaggcgaataaagcatcaaattacatgcacatcaggaatttaaaagcaatcatttgtgtcacctttgacatcctgtatgactgtcaattttcttaatcctttatattttctagtgaaagagagaacattatatcttactttcttctaatgaatatcattgagtaaatatttatataaaactatttaagtttaaaatatctctgaaaaataagaaacagtgataatatcatttattaaggcaaacaaaaagtacgtttgtaaatttgacaatctttaatcatttatttcataatcataatcgtggaggaattgaaaaacagaaagacctgctatcacctcacaaatgaaatacttagttcattgtgttttattctccttgcaaatcattgacgatatttaattaaaaattacaaaactgctagacattccagtcaaaactagtttcctctcttacaagcaggcagatcaaaactgactgacatttaatattatttgtttaccttcattttatctttaataaactcaagaatcttaaaaagccaatatatacatggttaaacaaattactgaaaaatagaactttgaatttatgattcagccttttccaaagaaacaatagcacaagcaaaaataaaaaatgagtgaaacatgattacaagtgttttagaaatacacgatttttcagctgtgtaggcacgtagtttatatcaactatcaacaaattgttcatgactttagaagggcaaacattagagtaggattagattgtggaatttccatggtttaactctaagaaagcacaggattggggttggaaattttactggaaagagttgattgtttagactgttgatttagttatgtgaaaatgggaggaggtagggtaatagctaaataggaactagaatataaaatgtaaacatagagttcggttgaaaatagtgatcaacagaagtggcagtaagtaccccagaattcctcagcttaaaaataagaaacaaatcaacaaaacattttctactttaaccacaaaaaaaaagcaaaatgaacaaagtacccattcatataagacaactaccacagaaactaaaggaatgtcttaattcaaaacaaggaatttgattttggttcttacttgccagagatcctaggcaagcatactcataaagtgaggaaatagagtcctaagagattaatacctggaatttcattgttagaaaaatttgcacaatttcaacggaatgaattggtgaaagctactctatcacataaatactattcaaatagcaatttacagtgtatgctgtgttccatatcctaggaaatctgttcattgttgatagggctgacggtctataaggaattatcactccctaaaaggaaagaaacttgcagagcaattcagcatccaaggaaagactgacagctttgaaagagacctgataatgatgcaagtaggaacttgcatgtgcttgaagtaagcactgatttaacaaaacaaagactctgttctgcacatactttggaaacacatattctttgagaacttggttatgacgagttgaaatggccacttgaactaaattcccatgttgagatttgtttatccttcttatctgaagccagagccaacaatcctaaaagttttgagtgacatttaatggttccaggaattggggcttacctatctgcagataaacctgcttcctttttggctgtattatattaagtaaaacagttctgttaaatgaatataaagtggtcccttctatactttagaataaataaatgacttgagccagatgcagtgactcacgcctctaatcccagcattttagaatgctgaggagggaggattgcttgaggccaggagttcaagagcagcctgagcagcaaagcaagatcccatttatataaaaaaattaaaaattagccacacttggtagcatgcgccttgtagtgccagctacttgggaggctgaagtgggaagatcacttgggcccaggaattcaacattgcagtgaaccatgattgcacccctgcactacagcccaggactcagagagagagcctgtctctgtaaaaaattaaagcataaataaataaataaataaataaaaggcttgacttttcccacagagatattctgctggtaaatagagaaataaagcttagtagtttgatttattttccatgacatcagttactctgagtacaatatttttgttacaagttgtattaaattatttgtgcttttaatgagccaattcaatgttattcttaataacaatgttatttttgtcactgcagcatttcagcagtgttcaaaatacatcagagtttccacttaaaagatctttataagagaaaagataacacattaaaatcatataaagcatgcaaacttacaacctcctcctcctgttacaagagtccctcctcctattacaatagtccctcctcctcctgtcacactagtcccttctcttcctgttacaataacccctgtcctcctattacaacattttaagtaatgtaatattaattttaaaaatctggccaggcacggtggttcatgcttgtaatcccagcacattgggaagctgagacgggtggatcatttgaggtcaggaagtttgagacagcctggccaacatggtgaaacttcctctctactaaaaataaaaaagtagccaggcatggtggcaggcacttgtaatctgagctactcgagaggctgaggcaggagaatcacttgagtaactaaaacgatagctttgaagagtactccgagttttatggcacttacttattaaaatagctgttttgtctcttttttcatatcttgcagccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggtaactttgtgttcattgggattatttttcattacgcttctctaaaaacccatgcttcttggtgctgttggggaaaatgaggcacctttatttatgatattttgattgtataaacttcaaatttaaaaatcttgttcagatgagcaaagaaaacaagtatttgcagttatactgcaatactgaagtgcacattcaaacttaaatgttgtcatctaatacaaaaataaaacttatttacattacaatgcaaaatcagtcctgcctttatctttattcttcataagtgaagtccttgaaacctttctattaagttacaagtgttcatttaaatggaaaattgtctgtaataaacacatatccttttatgcataatgtaatcaccccataggtcacagcaaaggaatacattttggctcctcaaagtcattggataactgatcatcaccaacttcaggatatgggcatgacatatggaataggcaatgtactccagaaaataagagtgagatattggagtttaaattctggctctgtcgctaatgagttctgtgagcgtaacttctttgtacctcactttctttatctttaaaatgggggcaatgccagaattcatctaacagggtcgacgttaggttatttgccatgagtactttaacaccctacctagcaaaataaatattaataaaatttgatgttttttcatcacactgtgtctatagcaaaatcatgcttaggagacctgatttggtatacagttcaaatggccaaagattagcttaggagacttatgtaagtttaaattcaaagttttaagcaggattctgtaccttaatacctaaatgctttttcctgtgtcccatctaaattccagctattctgtaagacccaacttcaatcccatttgtactatcctatcttataattcttaccatactaataatccaatcactacctttaatctttgtattattttctaatttccaaagtcagaatgtggtgttaaccactctttcatattactactgcatatagtgtgccaatggcattcaaaagaagtagagatcacaaaaagtagagtgcatcacacaaggtgtcacaaaggaaataacatgtgcattagagccttaaagatgagttaggtaaatgagttaggtcaatggtgatgttgggaagaacactgcaaacaacaaagaatgtgcatttactaatcatggcactttggtgggaacccaggagattaccaattatatttctcagttgttttggagattgagttactgtatataaagcacttagaatagtccatagtaagtaacagatagttgtttattttggttacctacttttctttataatccattatgtatccctacaacatagctttgatttgctttggggaattttatattaattttatatcaattaatcataccatttttattattttgggacttgcctctttcactcagctttaggtttagaagatgtatccactttaaagcatatagctgtagttaaccttctcttgaattgaactccactaattcaatatatcactagttattaagccactctgtctggattgacataagtgatacagatattgctgcaaaaaaacttttttatatgtctgatggtatatatgtgcaatagtttcagtaggttatttacctgagaatggaatttctgagtcacaggatggtcacatgtctaactttactggataatatcaaatgtaccaacttacattatctcagcagtatgtaagagttcccttgccaatatttagtatcatctgactttctaatttttggatattagagcatgcattatggtgtggttttttttttttttttctaaagactggaagttcttgtaatggaggggtcttttaaaaataaaccctcatgtttttaatgagcatctctaaagaaaaatatccccagcttgtttttaaggttatttattttattttattttttgccatgacattgtgttcactgccccagattcaacttgtgatcccactgggatcactaccctgcattaccaatctgaattacatacgttaaaacagccatctaaaagtgctagttgtaagagtctaaatacttgaatctttgagagacatatttatagtccattatcttcacctcagttaagtctgaagactatttgaaaaatgtaatcctattttttcttctaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaaggtaagacatgtaagcatttccagttcaaatgtaaacaacaaacttaaatcttccctatgtagtaagaatctacctctgtgttaagctgtagcaagatacatgcatgtacgtctaataaaaaagcagatatcaatagcacagaagaaactaatgattgtagatttgtgggtttctaacccaaagcaatattcatcagtttcaaattagtgagctgtttgtgagtaaacaatatatgagatggccactcataccttttctcatctaatatttgccagtaattacaattatattattctgtacctccagtctacttaatggtccttctcaaattttttcttaaggatctaactctaagtcacaaattctgatcctccccagttactctgtattaagactcttttttcccttcactgggtccctataatacaaacctggattcattgctttctttttttaatgagaaattaccatcaatgtgtcattatttgtatttattaacatatttattcaataagtatttgttctacaaggtgataatggtggatttcttagtaaaatagaatagcaggacccagtatgaaataaataggatctgaatactctagatttgggaagcagacctggcaagtaatctggtattgcacttcacctttcagggactctataactcatacaaatcaccatataacactgacacattattgctttctatttagattccagagtcagtgtcacaaaaccatttatgttaccccctgttgcagccagctcccttaggaatgacagcagtagcagtaatagtaagtacatatatctgatttaatgcatgcatggctccaattagcacctataggagtattgcatgggctttcaaggaaacttctacatttattattattgatactgttctgttactgttattccttttatggtcttcttgagacttaagtttgtagaattaaatttccctagagctggagataatgtttagagaattaggccaataaattttctgctgaggttattttaaataagacataaaattaattttagaaatatgatttatgccttttgttgaatcattaacatatattctgtgtggaatgtgtctgacaaaaacggaagtttaaaattaagtgtataaatgctggttgttaacatatattctgtgtggaatgtgtctgacaaaaacggaagtttaattgtataaatgctggttgtatagaattccaaagatattttcagatttgtgtatatcagtatttactattgcgcatttggaaatttgatgattttgttattttaccacagaattatatgcttgatgagaataggcttatttttctgaaatttctgtcctcttttgccaagaacccaattttctttgaaaatccagacaaaggtcattataaagtttattgtatttactaaatactctctagcctcatggaaggtgcaggaagaataaaaaccatcttgtgatctaggctaaaaaagtcatcccttaggacttataaatggggcactgtcatttgttcagggacttagaagacctatctgtgaaaagaagtaatcaatttattactaattcataatagcttgttacaatgcagaacatttaaaaaaactctaaaatacaatttacttattttggaattttttgccaccaaaacctgacctgaagtgatttgaggctatttatagtctatttatctttctaaaagtgaatatatattttactaatgaaatattaatgtatttgattacagaatgctacccaggattcactggggatagtatatgttatacattatgtgcaggacagaatcttctgaaacctaaaattctaaattacgtagcatatcagttctaaaatgtgtcaaaaagggactagagccctgtttgtgtgttagtcaattaagggttttcttaccaactatggattttattaacattttcaaaggagcaacttttgtcttcttaaattctctgcttatatttcattgatttctaatctttattatttccttccatttgctttgtgtttcatttactccactttttctagcttttcgaagtggaaaaattagatcattgattttaaactatttttgctaattacaagcacttaaaggtataaattttcttaaatacactgctttagctgcacctcacacatttttgtgttgtgattttattatttttctaaatatttgtcaactgtgatttcttctttgattcatggataatataaaagtgcgttgtttgatgtcaaaaagtttgagattttcctatatatcttattgttactagtctatgaattttgttgtcagaaaacatactctataatttttttgaaatgtactgaaactattttatgtatgttatttaactgttgaatgcagcattcaaatttggccaaaatggttgatagtgttgtttatatcttcccttccttattaagctttttgtctaatttttattaattattgagaaaagggaattgaaaatattaacttttgattgtggatttgtctgattttccatttggttttgggttttttgcttcatatattttaaagctttattattatgggcttatacattgattattgttagatcttccctgtttaattgaccttttaatcttactgaaatatccctcattatctctgatagtgctttttgtcttgaagtttattttgtctgatattagagcaaatctttcttatgcttactgttttcatggtatatgtttggctttcctttattttcagtctgttgtctgtatttttaaagtacatctctagtagacaacatacacttgggcttcactattttatttagtgtatctctgattgttaattagagtgcttcatccatttctatgtaataaatcattggtatagttggattttaatccaccatttccttacttgttttttatctgtcccatttgtgtattgaccttctgttccttatttctagccctttttttttggttaattattttttttctagtgtttaattagtgttggattttcaacagcacccttttgcattattttgtggttattttggaaattaaagtatacactttaacttgtaatattctacttagagttactattgtagcacttgagaaaaaaatgcaagaaccttgaaacagggtagttttatttacccatacccatcttttgtgtcatcattgacatatattaacatatctacatcagttataaatttcacaatacagtctacaatacacaatgcagttcaattatttctttgaactgtcatacgtattttaaagtaagagaagaagaaatatattgtcttttatattaacctgcatatttattatttcctgtacccttcattccctcttgtcagtccaggtttccatttggtattaatttccttcaggctgaaaaacattctatagcatctcttataatacagatctgctggcaacaaattcttttttctttttaattggaaaatatctttgttttgtcttccatgtagcaggtactttttactagatattgaagtctgggtacacaggattttttttcttttagtcttttaaaaatatcactatagtattcctgcagttattgtttctgaaaaaaataaaaaaggtagccaccatttgtcttatttttaccctgtatgtacagggtcatttcttcctctgcgttctttcaaagtgttctcctttttaacatttgagctagaatatgcctatgagtggttttctttgtatttatctttcttgagatttgtcaagcttttggatctatatatttgttttccctcaaatttggggaaaaaatagtcataaatttttcaaattttttaatccattttatctatcctctcattctgagatactacttgcatatatgttagcagattgctagtgtgtcataggtcgcagaagttttgttcatttatttatcctcttttctctgtgttcttctgagtaatttctacttgtctgctttcaagttcattgaccttttgttctgccatctctgatctgctgaatttttcatccagtgaattattctatttatgaatttatattttctgttctagaatgtccagttaattgtttaatgtatttcatatttctctgctggaattttccatctgcatactcattatgaaaaaaaattcttttttgtcacttaaatataatagctgctctaaagttcttgtctccaaatttcaacacataggtcatctcagagttgatgtctattgacttcttcctctctgattaggggtcacatttccttttaaatggttagtgattgtttaatatatgctggatattatgaattctatgctgttgaatatctttcagattctatagcgtttctttagaggctattgaattgtgttttggtaggaggttcgaatagtagcagatgaggtttatcttttctcccagccttgtttttaaactttcttgggtacaagggaaagtagccttattcctaaggcatggaatatctgagatatctgtaaagtgctcagaaaatctctccaatattgcttgtcttaataattttgtccccattgttctgtgtctgctagggcctctactaagattatagcttcccatgagtttggtgtttggttatttttttttattataattttattttttggtcaggttttgtgacttctctactggtgcatatacagcttattattggaccagtaacttaaggagattcatctagggatttctgatgctccttctttgcacagctcccttcttccaaataacctagatctagaaactccagctgcatcggcagccctgaacttcaatctctgactccttagcttagtgaggccattggtccttgtttgaactctgtcttccacacctcaatctgaaagtgcaccccagacaggagagctgaaagccagaaaaccagggcaaacatgagcctcacttagtgtggatgccctgtctcgtgaattacacatttgtactgctgttcactaatttctgaaaagagttgcctcataatttgtctagaattataattacttaggttggaaaggctagtctgctaccatcgtatttggaaacaaaagtctttcctactaattttaaaagacaaactaaatataaaatacctattcaatcccgtgcctctgaatctgagattactttttaaattgcaagatgaatggacagccttcttttttgttcactgtttcttttttcttcctttttaaaaaattttgagtcttgcattgtcagcaacttttagctaggtgatgtcactcatgaatatgccacttagatacctctgcacaggacaagattttgtggtgctaacattcagagagaaacagacgtaaataaaatactcatagtacagtatacaatatgtatcataatagaattatgcaaatgtttttaggtgtagcacaaagggggtaagacttttttgggagaagggttagggaaatttgaagaaagcatgctagttttcagggataactcaaaagctggccatgtcagagtaccagggcatatgaaaacttattacagagatgtgagtgtgtaaggaaaagtcatgggagaatggaataacatacaaacctattagttatccaaagggaattatgtttatagcataacagtaatatcctttatgataagcaaagagaatgtttaaaagatgttttatgtttgagattcttgcagacagtacctaaatgatagagaaagaaatcctaactttgacattatcttatacattttctagaaatagtcttgacccaaccttcctggactcatataccagtcaagttaaactcttttgactcttcggtcaaatggaagatcataatacctccttcacagagtttctgtgagttttaagtgagttagtacacatgtgcttggagacataactgatgtcttataagagttcatcattaagtactagaacaatgagatgcaataagactttggatcagtaattatgttgaacctttagtgagctctggatctgaatcaaggaagaatactcatttgcttgtttcataagcaccaagataaccatgtcacaggcatggtgctcctaaaaatgctcactctaaaaagaagttcagcaaaattatagcacaattctttgtgttctgtatttctacagatgttggttattattagtcaaatgaaacaagtcagttcccaagatttctggacagtgaatagttttggtaataaggagatataattaatatggtaataacccaggaatttgggattactcaaatccggcccagatgagcttcctctccacataaattctctgcaggctatctttgcataaaatcaaaactttgttgtcagcttaagttttgtccccaaatacccttcatttgggccttgaggcatgcaattcaaatctcattgattcctactcgatagctgcagtggaaagtattgaaagtcattccctggaggtataagcaaatctcttctgataaagttctttttgtctgcctgcaagagggacagtagtatccctgatggaccaaaggcctggttcttaagccccacgcatgaacaagtgaaataaaaaaagtgaggtaacattgacctctatgggaggtgaagctcagtaacctatgggcgcttcccccaaccctcctctccactgccgccccgccatccagtctttgctgtttctctccctccctgtctcctgcaggaagattgctgctgtgtcttagtcagactccaggattgtgtattggctcacagtcttagggatggaactgtcaacatggtagtcaccgcactctcagagagcaagtctgagctttctttagtaaggcctcccacacagctttgttggaagccccttccttagatgctatccttctgcctgagaaaggttttaggtagcaaattccagaatatctcactctttagtaagctgctttttgcttttgagtctggtctttcctttggtctgagtggtgaatttcaagacttaggtttggtctttgtatagaacaagaagaacatggtgtttggctaccttgattacagggcatctggctgacccaacgtagataatggagttctccatagcatcttcctagcttgttagagttttccattggattttaaggacaaaatcttaattaatttatttgttctctctgtgtacattgattatggcctcttaaggaggttacctagtaaatctattgtaattacataccagcaatcccaatagataatgggggccacagcagattggccttcaaggagaaccaaaacagccaaatgatttatttacaaacagtattattaaaattcctcaaatggtgcgaattgtttgtaaccctgaatccatttcatatagagagcacttttatagatagtagttaaatcccaaacgagtccacgtaatcttacataatccttaaaatagctctgttattgtaatgatagtactttaattataccaaatcccattttattactcagatttattttggtgagaaaattcacattgggctccaacttgagatggtaaaacatatctcccatggggctataggatgtggggtgggccaggaggaggaatcctaaggaggatattcatggttatgtgaatggaagaccctatttctgacaactccctacgtggggatcaccagcaccttattgttcatccctcactgtctcctcccccaacacacacacacacacacacacacacacgtgagcacacatccatacatcccatcattagagtctccttggaaacataccatttataggatgagggaatttcctgctctcagttcacatatggtagaggaatgggctgttcctgtcctcaccatagtagataaaatctatttctgacttatcaagttaaatctatgaattaatttttccctgtaagatgttgatacacacatagtagatctattattttatattatgtactctaggtactttgtatgttaaataggtataggtatatagaaaaatctgtcaaaataagtttcatgttggaagagacccttgtgggagatgtgttttgtttatttaattcgtgaatcattctgctccttttttttttttaaagacagagtcttgctctgttgcccaggctggagtgcagtgacatgatctcggctcattacgacttccgcctcccgggttcatgcgattctcctgcctcagcctccgtagtagccgggattacaggcatgcgccaccacacccagctaatttttgtatttttagtagagacggggttttaccgtgttggctgggctggtcttgaactctggatctcaggtgatctgcttgactcggcctcccaaagtgctgggattacaggggtgagccaccacatccaacctaaattcatgaatcattttgcttctcatggaagagccaggcaggatcaagaaaggtgtcccaaaccaattccatatatgttctctctcattgagtttcttatttagacgtagtatgggacaacactagtgccttcaaccattccccttagtgttagtcttaagctttccaacattaaaagtccaaactcttagtccgttgcacagtggcatccgacaaatgtgtgttgaagatatgaacgtggaaacagccatgattctaagcatgagatacctaaaaatactataaccacagtgcacatacaattatttaatattttcaacagtccaaacatagctgaatgcatacagatcttgatccaggttgtaaaatgtcatgtttaaaatttctgagcaagctgtcaagagtgagaaagcaaaaagattttagaagataaaggcatgggagatagatttgcttgatagtataagtcattttccccccagaaatatattaagtctgtgttccatatgtgtcagaattacccaagccagatctctttctgtctctgtgatttctttcttttttaatgcattcatcttttggagactacatggatttatattgccttgcaaaagcaatattcagcagaaaaatcaatcttttgaatactttaaacagatataggtccagaatgtgttacagaaacagttaaaaacaaccacagcataagagcaaaacttctagaatggatatgctgtattcatcagtgtgttctttaaattatagggaaggccaaaaatccccctggagactccagcctacactgggcagccatggcattgccagcattgttttctcttataattggctttgcttttggagccttatactggaaggtaagtggtaccattcctttttttaaaaatatgctatgtttacataaattatcatctttttttcctcaagaaatgatcctttaagaaaacagtgaattctaccttagcttataatctaaacaaaatttaaattttataaagtttcctgtttctcattatgtctggagacaatccctctagctgataattcacgcttaagaattaggaactggtgttaatagcctgaactaaatatgaaaaggaagatctgacgaaaatcatatgatatacttaaaagacaaatagaaatgtcatttgtattatctgctattttggatttctactcttttgttctcttcttattgaagcctgtctaaaattttaaaatgtgcaaaaatcattgtgactcagcttcttgaaatgtcttttgaatttgattaagtgtcagcttctttattactctttagcccccaacatcctaaaaatgttgagttgatagtaccatgcagctttcataattttttattacaaagatattttgcatgctacatgctgaaaaaactgttattggagttattgccataaaagataaaagtggagtccacttacctcttaaatattagaccattcattgattattttacagtatatgtctttcttctttttccagaagagacagccaagtcttacaagggcagttgaaaatatacaaattaatgaagaggataatgagataaggtattttgttttgctaaatgtgtgcccaatcaagcatgacattgccatttcacacactgtgtacctgcccataatgtctttaagaagtccttcactcatgacagtagctcctaaccagtgagtcccaactctatccatgtttctgatgtctcactctctcttcagtgctgcatctccatttacctacagaataccttcacataattgtcttcatgactcttcttagggtttgtaaaggggttacctctgctttcttatttaagtaaggcagcagaatcttctcagactcctggttgtggaactctggaggcacatttgctcatgttctgtcttttccacacttttatcaaaacatactgattcagctcttggagcgtcttcctaagcttagttctttcagtaggaagcattatcatcactgacatctttatgatctcatgagttgctgccactgatcttttctttagcccttctaaactcaacctttagtcacactcttctgtaaaccctggttcatagtgtgacttcttattcaaaatacgttcagtgattttctcttgtatattttatatatcttttattttactctggctttcagagcccttaactattctcaacatactcgttgttttctctaaagctgaatacaaaatttgctgtaagatgactttccattcactgtagctggctcttgtcattctttcaccttaccctatactgcccagcactcatatgttcccttacctttgattataattttcatttggtgtgttgccttctctcatgtcatgcctgtatgtatacacagacacatatgaaatgcatataggcatgcttggtatgtgtatatgcatatacagagaaagaaatgttttaactacttggaaagactaccttaagacaaatgaagtcttccctcttccctatagtaataagaaggtaggctccccattcaattttgcaatcttctgctactatatttacagaaaagctgccttttacaatgccgagatcatggtgtacctcagaatctctgaccaagagcaaataagcattttttcttattgtttttcagtatgttgcaagagaaagagagagagtttcaagaagtgtaattgtggcttgtatcaacactgttactttcgtacattggtaagtttttttcttctttcctttttttttcttttttttattatactttaagttctagggtacatgtgcacaatgtgcaggtttgttacgtatgtttacatgtgccatgttggtgtgctgcacccattaactcgtcatttacattaggtatatctcctaatgctatcccttccccctccccccaccccatgacaggctccattgtgtggtgttccctaccctgtgtccaagtgttctcattgttcaattcccacctatgagtgagaacacgcagtgtttggttttctgtccttgcgatagtttgctcagaatgatggtttccagcctcagtcatacatgtgcatgtgtctttagagcagcatgatttataattttataatcctttgggtatatacccagtaatgggatgcctgggtcaaatggtgtttctagttctagatccctgaggaatcaccacactgacttccacaatggttgaactactttacagtcccaccaacagtgtaaaagtgttcctatttctccacatcctctgcagcacctgttgtttcctgactttttaatgatcaccattctaactggtgtgagatggttatctcattgtggttttgatttgcatttctctgatggccagtgatgatgagcattttttcacgtgtctgttggctgcataaatatcttcttttgagaagtgtctgttcatatccttcgcccactttttgatggggttgtttgattttttcttgtaaatttgtttaagttctttgcagattctggatattagtcctttgtcagatgggtagattgtaaaaattttaattcaaactgaaatatttagcaagaactatacagcatatgagatgccaaagtttagaaacaaacttcattagtaagtcttctatcaagcagatgtcagtatgttggctgaagctgttacataattgaaatgtgcatatctaattcattgtgtattctccagttttgaaaggtaagcagtgtttgtcctgactagtggattcatctagtgtgggatatgcacaaaaattaacagttgtatgtttacttagactgtttttgagaccagaaaattaattagacaagggaaatatagcaaattaggactaaaattaagtattactaatagaataaaaatatatggaatcaattttatttaggtgtgaccactataacatgactgtatcccatgtgttagatgctgaaacaagagaaaaccagaatgtttccctgccattttcaggaaaaagagaaaacatccaaatatcttagagtcaggagagtaagattttccaggtcaattgccaccttccacatgactagtaataattatgcattataatagttcagagcccaggatgcacggaacataaacgcatggcagacaggtgctgtgactgtgtgggtctgcggagggattgtgggagaaaggagagtaagatgtggctagaaggtactggtatcattatctcctgtcccgtctgctttctctgctcccttgctgcagatactgttgcgggaagacccacagctgcattaacttaatcagtattgattcatgctgtccctcacttttgcttctgctaattggctgttatttttgttactggcatggaatcctgagagcatttaccacagttgcatgcgaatattctctcctctttcaaattcctcatttcttctgtgcattccctcattgtcaaaggattatttactctcctctttattacctagatagtctgtggtggattccttctttaccctgcaagtcactgcccttccagctttaactcctgtcccaggttcagagatttgtctctagacttaatcttcttcagtggaactgagtcataaattatcttgcctcctgtatgagcttacttttatggttatacagcaacattttttaacaactcaagataacatgatataattatgttgcacatttctctaacactgtaatactttattctacacactctgtcacttttaccttcaatcatactcttgattaacatgtccgtagcatgaatagcaccctctattttctgctgcccattgccacttctttctaggctcattcttcctgtcaaataatgtaaatggatagaccgtttgcttgacactttttattttccgaagcaatatttcccaagctgaaaccatgctttttcctagaaattcatttttttgcctttgaaccataagatgagattcactgttttcttttttttttccttccttttcttcttcttgacagccatttcagttctctcaagtggcattcacaaaccacacattaaatggttaattgagtcttaaatgctcaaatgctcaagtaatgtcctctgttgtttggtttgggattgcattgctgggatcctgttttggcacacaggtctttaatcatgtggttttaattacgacattcgtcccatgacatttgggagtggattaggatacggaaacccaggccatttgtttaggaggaaaaaagatggtattttaaagcaaccatagtcaaacaacttatgaatgtgttgaatagtcagttagcattccaagaaaagagttaggtctttctgtttcttgctgtaccctcaacacctaacttgctgcccgtgcattgttggctctctataaatgtttgttgaatgagagactgagtgagtgactcagccctaaatgaaagtttaagcaaatggcaaagaagattgttcttctgaagtagggtttgtttttatcttttgtgtgaatgtgtgtatttaatgtattgggttggattaatgagacagcaaggtatgcatatcaagggggaatataatcacatgagaaattaactaataaattatttttcttaaggtatgaattattactcatgtataaaatgaatcacattagccaggttaaagaagccagaattatccagaaaaaaacccagctcacttattcagtagtcaatgggaaatagatggcagaatgaatgaaaacatgactcattctccttcacaaagtgccttactcgtcatctctgcaaagctccaccaaggctcaggtcacatactgccacctacaaaaacctgacttcccaaatgaaaataatctctatttctggccctctaaatagatttcatatgacacttggtaggacatcacagtcacctaaatgaacattttgtgcccttttcaaaaccataaactcttagggggcaggatttttttttcttaatttagttttggagccccactaaacaatgtcacttatactgtgcacatataaatgatgaataactatttgttgaataaattaatggaaagatgaatgaatatctcagaattacaataagatgagaataaaatatatgaaaataaaaacagaaataaacttgtatgtgtctatgagtacataagaattcaaatgctgagggaaaagtaggaaaagaggtagaaaagggccagttcgtcctcagaatctgaaaaccccaaagtaaaaatgtgtgtatatcatttgtaatcatctattgactacccattatatgaagtaaacccttacatttggccttctaccagcagagatggaaaaccctccaaggaactcttaagtctagttcctagataggtgtgtagtagagggaaggtctgggagaacaaactgtgatgtggttggagagaaaatcttctaaaaaataagaataaccgtagtaaaaaatgccaagacagaaataaaatgtggagttgcaattatacatttaaaaaatagatataatttggattataatcattttcttggttaacaaggaaggggaaaaatacaaggaatgactcaggtttatggcaaggatttctccctgaaatggaagtttcaccaactaaaacaaattaaatcaagacaaatattggaacactagaatagagaacatatgggctagttctcttaaggaaataacctataattggaaaatgaaaagaaggagggtttaatatcttaactacaaggaacaaacgtggaaggaatgtattgaactagaaacaacaacaaaaaaagttgcacctaggaataaataggccaggagtggtagctcattcctgtaatcccagtaccttggggggctgaggtgggaggatcacttgagcccaggagttcgagaccagcctgggaaacataggaagaccccatctctacaaataatttaaaaattgggtgggcatagtggtatgcacctatggtctcagctactcaggaggctgaggcaggaggattgcctgagcccgagaggttgaggctgtaataagccatgatcaaatcacagcattccaacctgggtgatagagcaaggcctggtctcaaaatataaaaataaaaaattaactgaagtgtgtttatttgacaccatgcgtgagaacagctatgtgaaaagttaaaaatttacttggtcacatagcttatgccaaatatccatagtagcatcctacaaaaacctttttcatattgggttgtggatagcaaagattaagatagataaataaaacagatggattacattaaaaactttatgttaatacgttacaggaaacttttgctctttactcttcccacaaatgtgattccaacaaactctagcaggtgcattgatggatgcaaagcttaatataactttgttgtgtcctcaaattgtttatagcctacaaaaggaaatcagacatatacataataaccaatttttaaaagtgaactaaaagtttacagagttactttgatcaaagtccaggcctctgcatatgttcatacaagttgtgaattcaacacagttctagatactggaatgaatgacatattagaattgagcacacccaaatacagttacctatttttgaaaggagagagaaatcactaacaataggagaaaagcttcactttgtaagtggtggaggtagcatctgtactgagcacatggcgtaaactaagtcagtatagtcccaaagtagaaactgtgttcaagggtcagtttggacagttggattccagtggaagtcacatataaagagttaaatgctgagaagaaagggtgagcatcggtttgagaaagggtttgaattccaggaatggggcttaaatgccttgtgtcacataaggaggtattgggagaaaatggtgtaggaaagatgtgggtgacaatcaaagctgaggtgtgagaaaactaatcgggtgttggtatggcaagatgtacgggagtggttaactagagaaattgagtccacttaccaggccagtctttaaataagatgtgagagagaatgacaactgaattagcatcagggccaggaaataagaaagcatagaccatttgaagacattgcagtaaaagaatcaacaaatcctagttactaatgggatgtgagacttggcatcacaggcagaactcaagaatgatttggagactatgggacttaccagagtgcggtggcattaacaggaaatggaaaatcaggagaagaagctgttggtaaatattattgaaactaaccagtggccaaatatgttagactggaacttcagataacagtcaggcctggaaagaatgatttgcaatttgagagttatctgcaaagaatcttatttgagaccttgaaagagaaattgcttagtcacagaaatggccccgtgtggttgatagtaaatagcatgggtttgattgtggagaggttcactaaagaaatacaaacaaaattttggtatatctgagctaagggggaagaaaataaatcttagaattggaataaaggacaagctgaagacaagcttggaaatttaggcctcaaggcttggcaatggttggttaccaaatagcaagacctgtgattctttgctagatgtttcctaagactttcaaagctttgggagtttggtttctgtagatacattgtaaagcagatgattagaaaggattaggcttggattcaagccttgactctttggtgtagaatactagatgaatttatatcagttatcagttcatgtcttttaaaccctggtttttcatgtgtaatgagggttagtgataaatgtactcttcttatttcacaagggttcttgcaagaacccaggttatcattataaaaattgtagttttgttctatgggaaaacaggtgccagtctttacgtttattttctttgctaagtgacccggggacaaaaatcaaaaatagatttccaatcttctctacatttaattctctctctctctctttctttcttgagagtctttctttcttaagagtcttgctctgtagcccaggctggagtgcagtggcgtgatctcagctcactgcaacctccgcctcctgggttgaagggggttttgtgcctcacccccagagtagctgggactacagatgtgcactaacacacccagctaatttttgtatttttagtagagacagggtttcaccatgttggccaggctggtctcaaactcctgacttcagatgatccgcctacctcagcctctcaaagtgctgggattacaggtgtgagccaccatgcctgtcctatatttaattctctatattaagctttgtgtttccaattatttatacatttaaaaaatacatccttttgctttttgtgattcccttttgctttctaaccatgcttttaaaatatcttctatatattttggatttctgaagttttatcttagtatctcttaatttttgttctagatttaattctgatttcctaaatacaggttgtctagcttctagagttctctgcttggtaaattgttgtataagacagtttttaatatattacatttagagagactggaaaaatattgcaattcttattacactatggaattttatggctaatctgtgtaatgatctcaatactttaaaaacagtgatctgtgatgctgggttgataaacaaggacaaaagtagtgaaagagattgagcagagaagacgagaaatgcaaaacacatttatttagagactgggattgaagatgggatgataccatctatatgtattttttttaagtagagaaggaaatacaaatactttactggaaagttgaaacatctgaaagctagtagattaacacaagtactgaataactaaaacaaaatatggctttcatgcttcaacaagtacaataagctatgctgaggccaaacgaactttccccagaactttgttgggatgcggtgccaagattacagtctttctttttttggttggaagtattgaacgaaatctttctgctgctatttcctcagctggaagaaatgattgctagaacctaactagatagtcaagatactagagcggctcagtggccatgtgaggctattacttgttggtggccattgaccagcttctccacctgtcttagggatctctttcatgctactccatctatctccatttctctaatctccactctccagacactttgttcgtcaatccttctcccttcacccctgtttactaccttttataatcttgctagagaaattcaaagctaactaagttgataattatattatcacaaactgagatcattggtagaatgtcaggaggtatgaaagtcacagggtgaaatatttattgagtgtctattaagtgccagacattattataagagtggaggtacagcagtaaaagagatagagaagctccctgccctcatggagcttacattttaatgggaggacatagataataaacaaaaagtaagattgtttcagataatgtgttaccaaggagatcatagaaaatgactgatcactgcatctaagaaaatatttatgaagttcttttctgctatataagtgaagcatacattgcaaatttgttactcattctgtggtcatatatgatttcggctattgagatttttaaaaaggcagaaggcagatacaccataatctctcttttcttccttcctttctaggctggtaacagttcatgtttgcttcataaatgaagcagctttaaacaaattcatattctgtctggagtgacagaccacatctttatctgttcttgctacccatgactttatatggatgattcagaaattggaacagaatgttttactgtgaaactggcactgaattaatcatctataaagaagaacttgcatggagcaggactctattttaaggactgcgggacttgggtctcatttagaacttgcagctgatgttggaagagaaagcacgtgtctcagactgcatgtaccatttgcatggctccagaaatgtctaaatgctgaaaaaacacctagctttattcttcagatacaaactgcagcctgtagttatcctggtctctgcaagtagatttcagcttggatagtgagggtaacaatttttctcaaagggatctggaaaaaatgtttaaaactcagtagtgtcagccactgtacagtgtagaaagcagtgggaactgtgattggatttggcaacatgtcagctttatagttgccgattagtgatatgggtctgatttcgatctcttcctgatgtaaaccatgctcacccatatcccactatacaaatgcaaatggttgcctggttccatttatgcaagggagccagtactgaattatgccttggcagaggggagactccaaaagagtcatcgcaggaagaagttaagaacactgaacatcagaacagtctgccaagaaggacattggcatcctgggaaagtccgccttttcccttgaccactatagggtgtataaatcgtgtttgcaaaatgtgttatgatgtgtttatattctaaaactattacagagctatgtaaagggacttaggagaaaatgctgaatgtaagatggtcccatttcaatttccaccatgggagagcctaaaaataaattatgacatttagtatctaaggttagaaaaccacgcccacatgctaatatgggtgttgaaaactaggttacttataatgcaaggaatcaggaaactttagttatttatagtataatcaccattatctgtttaaaggatccatttagttaaaatcgggcactctatattcattaaggtttatgaattaaaaagaaagctttatgtagttatgcatgtcagtttgctatttaaaatgtgtgacagtgtttgtcatattaagagtgaatttggcaggaattcccaagatggacattgtgcttttaaactagaacttgtaagacattatgtgaatatcccttgccaattttttttataataagaaaacatctgactaaagtcaaagaatgatttcttatggtttattttgatgaaagttcttttaacatgtcttgaatgtacacataaaggaatccaaagctttccattctaacttaatctttgtgataacattattgccatgttctacaaccgtaagatgacagttttcaatgtagtgacacaaaagggcatgaaaaactaactgctagctttcctttcatttcaaaagtccaagaatttctagtatatttggattttagcttctgttcaaagcaaatccagatgcaactccagtaagtggcctttgctcttttttgtaccaaagagcccagatgattcctacagtccctttcttctctaacatgctgtggttccttaaatatgagtaatttctctaagatataacccaggtgctttgagaagctgcattaaggtgttcaggccctcagatatcacatggtacacttgattagtaataaaaccagagatcaatttaaattgctgataggtcctgtctcagtgtgtggcattgactgttttcaggaaaatagatacagattaatatgagttatgcgtgtaggttgtgtatagattgagaagatagatacttctcaatctagtagtttgatttatttaaccaatggtttcagtttgcttgagcatatgaaaatcctgcttaatgtgcttaagagtataataaatgtgtacttttgtcctcaaacctagtagctgggttttaacactcatggacatggtcttaatcaatggagttaaataaacaaattcagcaagttattaaatctgacatggtaggagaggggagatgtgtcctgcttattaaatgtgttggtccattgaaagttacatggattgccaatttttaaaacactaaagttgaataaaatgcatgaacaatagaaaaatgctgaacattattttggatgctagctgcttggacattaactgtgttatttctgctttgagatgaaaatatatatttatctttgcttattttatcccagatgtgttctgaatatccttcttcataaatcatggaaaactcactgctgagatagtaaaccatgaaatcgccttttcagttggtgccatgtatctgacagttccatcttggaaggtttcaaaattaccttttaaaatgatctcagaagtctgtagattctcaatgatactgaaagctttgcacctctttggtagaaaccaggtctatttagaaaatggctttatgataaatgttgcctcctgagtgataatgaagtgttcctggatattgtattgtaatttaatgtgcttaccacactgccacattttaatgagtcagagaaaaattaatttttcttcaatacaataatagaacaagtagcctattctcttaaaaagtatgtgaaaagaaaattatgaaaaaatatgcatacctaatgaagtattggttttagtaagaattaaatacatttcattgagctttaaagtactttggagaaactttggggcacgttttcctactctaattcaactaaagttataaataaagagaaaaactcattcagaaatcatggattttaaaaatattttactgcagccaagttttcatttcaaaatgtaatttcagtttggagcttttaggcattatgtatatttaaaaaatatattcttcaaaaatgcattttggcatggtgggatggatgttgcaaaagatatccggagcctccagtctgtcattaactgatatggtaaatcacctctcttctttgggtctcaattttttatttatctatatggtaaactcagagatcactccttaggggtgagtcctattgcaatatgaccgacaaagaagacaaaatagcattgaaactaacccatacaaaatatccaactctggattctgtgaataagtatcttgaccataaaaagtcattgctgttcttgtttctaatgtaaatagtgtccattagtaaaagtgaaattcagtcttaagtagggtgaattggatcaccatttacacaagagatggctttttcctttgcttgaataaacattttggatcacctccaaagaatgaaaaccagtagtacgttttagtcatattagtcaggatgagaaactataagatgtgtgtaacatttggaaatgcaccaaagtgagcgtttaaatcttctcattttattgaaaactaagagcagaaaatgtaaaatgctcatgaaggttttgaatgccaaaagatattttagaatcaatttataaaggggtaattcattaattacactttaaaattggaaagtgggataagaaatctaaagtaaaccagcttatctttgaaacaatattattttgaaattggctttaaaataaaaccattcagattgaaattctaattagctcatttgtggagtttgatcacacaattcataatgttgctgctttccattaactagtcttgaaatgcctttgtttgtaaaaataaaataatggtactttcattttataacaaggtgtttttttcaagaaataatccatgctaaaatggatatttgtgatcctgaaatgtttactaagcattgtaaatttatttataactgccatctccaactacatccttatgatgtttttaacaataaaattaaaacaactgttaaactaaaaaccacaccgttttccagtacttgatctctgagctacaatactcactaaatataattttccaatcaaaatattctattctatattctaagggttaatatgtgattatagtgtccacttgccaccatttttttaaatcaatggacttgaaaagtattaatttagatggatgcgcagatataccctcagttcagtcatagattggagtttgcatataataatgtaaatgtatgtcgacactattctaaatagttctattatgactgaaatttaattaaataaaaaaggttgtaaaatgtgatgtgtatgtgtatatactgtatgtgtactttttaaaataggtgtatgtcccaacccttttttatacaggtttgaatttaaaattacatgatatatacatatactttattgttctaaataaagaattttatgcactctcataaatttaccctgtcattttatatcttaaggttatcacatatgtctacaacatggttgaacatttattaagctacagtcaaaattttctgtggaaacttttgtcattaatctagattttattaagctacagtcaaaattttctatggaaacttttgtcattaatctagattttgttgaaatatggacacaaggggataagaaattatttaaattcaataaatatttattgattactaaaataagtaatcaaggaaaataaaaaatgaattaacacgacgtttgctttcaagagatttataacttgtctggagaaaagaaatgcaatagatatatagaaaagcactataatagatatagatacagaaaatagatacagatatagaaaagcagtataatatgtatagtccctgaagtaaatacctacatgcaaaacagaggtccagttaatgtgcttagggcatgcaggaaggagaaatggatacagaataagggagactgtggaggaattgatgtttgggctaatacttagaaggatgagacaaatttttatagtgagaattatgcccagtcagggcagcattttccatagaaagaagttataaaatttctaagcttattttaccaactcaaacaggtatgtccttagcatacaccaaataaaaagacacacggagagataagaccagacccttcaatgtaacagtgagtgcagacactattcttagggtccctgagaagcctagactcctttgcttgtttctgagaagaggaatgacgagatcaaaacgcactacggatcctgtgatttaggcagtagataatcatcctgcttaaccactggttgctatttgactctcaagcacatacccttgctatatgacaccacacttattcacccaccatttcttttcagaatttgctgattctctctcctctgtttaccttttatattgccaacttcgaccaatatatgactagataactagaatagcctcctaactgatcacatgattctactttcaccctttttcaatcagttcatcacattatagtcaataatagggagacatctggtgtgtgtgtgtgtgtgtatgtgtgtgtgtgtgtgtgtgtgtgtgtgtgtgtttctggctaaaacacttggatggcttctcattattttttagctaaagcccaaactcttcaacacagcctacaaggctctacttgagccatctcatcttttttgttttgttttgttttggattgctgattgtttttgcctttaatccataaacaacatgaaggttgggcccataaatactaatataactgcagtcctaaaatttagtgcaaattattagtggaattgaaacaaaatgtgatctaaatagctatcacctgttgtactgagaagttattttttaagatttttattatggacaatttgaaatatatgcaaaagtcgataagatagtgtaataaattcatataaccataaccctacttcaaccttactcaactcctgaataatcttgttttatctaaactccctatccactccccattttgtatcattttcaggaaagtacttaaaagtatttgatctggaaacatttctatgtatatctctgttaactttcagaaagcgtaaccctgatacccttgttatacctaagaaaagttattaacaatttcttaataccaaggagccaacaagtatttacattttaactcttttacactgtacactgcagtattgcactagagtagggtataaaatacagttaaaagtcccccttgaacattaaagttagtttccctggaggtaaacactgctaattattcctcattaacttctagaattttttggcatgtacaaactatatatacatgtatatacaatactagtctctaactttatctctttcatttattaggagaacgcagaacactacatcagcctagataaatctttacccactgcaccataataaccaggaaattatagataggcattacga

SEQ ID NO: 8 - protein, 11 aa, Homo sapiens
EGICRNRVTNN

(19) United States
(12) Patent Application Publication
(10) Pub. No.: US 2025/0042986 A1
(43) Pub. Date: Feb. 6, 2025

(54) STEM CELL FACTOR INHIBITOR

(71) Applicant: THE REGENTS OF THE UNIVERSITY OF MICHIGAN, Ann Arbor, MI (US)

(72) Inventors: Nicholas W. Lukacs, Brighton, MI (US); Vladislav A. Dolgachev, Ann Arbor, MI (US); Steven L. Kunkel, Ann Arbor, MI (US); Cory M. Hogaboam, Ann Arbor, MI (US); Sem H. Phan, Ann Arbor, MI (US)

(21) Appl. No.: 18/731,458

(22) Filed: Jun. 3, 2024

Related U.S. Application Data
(63) Continuation of application No. 17/493,411, filed on Oct. 4, 2021, now abandoned.
Application No. 17/493,411 is a continuation of application No. 15/782,503, filed on Oct. 12, 2017, now abandoned.
Application No. 15/782,503 is a continuation of application No. 15/058,918, filed on Mar. 2, 2016, now Pat. No. 9,790,272.
Application No. 15/058,918 is a division of application No. 13/937,852, filed on Jul. 9, 2013, now Pat. No. 9,353,178.
Application No. 13/937,852 is a division of application No. 13/347,459, filed on Jan. 10, 2012, now Pat. No. 8,911,729.
(60) Provisional application No. 61/431,246, filed on Jan. 10, 2011.

(51) Int. Cl. C07K 16/24; A61K 9/00; A61K 9/08; A61K 39/00; C07K 16/22

(52) U.S. Cl. CPC C07K 16/24; A61K 9/007; A61K 9/08; C07K 16/22; A61K 2039/505; C07K 2317/76

(57) ABSTRACT

Provided herein are methods, compositions, and uses relating to inhibitors of stem cell factor. For example, provided herein are antibodies targeting stem cell factor and methods for treating fibrotic and tissue remodeling diseases.

This application is a continuation of U.S. patent application Ser. No. 17/493,411, filed Oct. 4, 2021, which is a continuation of U.S. patent application Ser. No. 15/782,503, filed Oct. 12, 2017, which is a continuation of U.S. patent application Ser. No. 15/058,918, filed Mar. 2, 2016, now U.S. Pat. No. 9,790,272, issued on Oct. 17, 2017, which is a divisional of U.S. patent application Ser. No. 13/937,852, filed Jul. 9, 2013, now U.S. Pat. No. 9,353,178, issued on May 31, 2016, which is a divisional of U.S. patent application Ser. No. 13/347,459, filed on Jan. 10, 2012, now U.S. Pat. No. 8,911,729, issued on Dec. 16, 2014, which claims the benefit of U.S. Patent Application Ser. No. 61/431,246, filed on Jan. 10, 2011, each of which is incorporated herein by reference in its entirety for all purposes.

### STATEMENT REGARDING FEDERALLY SPONSORED RESEARCH OR DEVELOPMENT

This invention was made with government support under HL059178 awarded by the National Institutes of Health. The government has certain rights in the invention.

### SEQUENCE LISTING

The computer readable sequence listing filed herewith, titled “UM-31623307_SQL”, created May 31, 2024, having a file size of 113,981 bytes, is hereby incorporated by reference in its entirety.

### FIELD OF INVENTION

Provided herein are methods, compositions, and uses relating to inhibitors of stem cell factor. For example, provided herein are antibodies targeting stem cell factor and methods for treating fibrotic and tissue remodeling diseases.

### BACKGROUND

Diseases involving tissue remodeling and fibrosis are a leading cause of death worldwide. Nearly 45 percent of all natural deaths in the western world are attributable to some type of chronic fibroproliferative disease and the associated health care costs are in the billions of dollars. Tissue remodeling is the reorganization or renovation of existing tissues, which can either change the characteristics of a tissue (e.g., blood vessel remodeling) or participate in establishing the dynamic equilibrium of a tissue (e.g., bone remodeling). Fibrosis is the formation or development of excess fibrous connective tissue in an organ or tissue as a reparative or reactive process, as opposed to formation of fibrous tissue as a normal constituent of an organ or tissue. Fibrosis affects nearly all tissues and organ systems, and fibrotic tissue remodeling can influence cancer metastasis and accelerate chronic graft rejection in transplant recipients. Diseases in which fibrosis is a major cause of morbidity and mortality include the interstitial lung diseases, liver cirrhosis, kidney disease, heart disease, and systemic sclerosis, among others.

Stem cell factor (SCF) and its receptor c-Kit have been implicated in fibrotic and tissue remodeling diseases (El-Koraie, et al., Kidney Int. 60:167 (2001); Powell, et al., Am. J. Physiol. 289: G2 (2005); El Kossi, et al., Am. J. Kidney Dis. 41:785 (2003); Powell, et al., Am. J. Physiol. 277: C183 (1999)). c-Kit is a type III receptor-tyrosine kinase that is present in many cell types (Orr-Urtreger et al., Development 109:911 (1990)). It is also expressed in the early stages of differentiation (Andre et al., Oncogene 4:1047 (1989)) and certain tumors exhibit elevated expression of c-kit. SCF is a ligand specific for the c-Kit receptor kinase. Binding causes dimerization of c-Kit and activation of its kinase activity. SCF was first isolated from the supernatant of murine fibroblasts. At the time, SCF was called mast cell growth factor (MGF) (Williams et al., Cell 63:167 (1990)) or hematopoietic growth factor KL (Kit ligand) (Huang et al., Cell 63:225 (1990)). A homologue was subsequently isolated from rat liver cells and designated stem cell factor (SCF) (Zsebo et al., Cell 63:195 (1990)). The corresponding human protein is designated variously as SCF, MGF, or Steel Factor (SF) (Cell 63:203 (1990)).

Previous studies have suggested that an inhibitor of c-Kit receptor tyrosine kinase can significantly inhibit aberrant tissue fibrosis (see, e.g., Aono, Am. J. Respir. Crit. Care Med. 171:1279 (2005); Vuorinen, et al., Exp. Lung Res. 33:357 (2007); Vittal, et al., J. Pharmacol. Exp. Ther. 321:35 (2007); Distler, et al., Arthritis Rheum 56:311 (2007)). However, this inhibitor has several disadvantages. It needs to be given systemically by oral administration, it has some toxicity associated with its use, and the compound must be delivered intracellularly for efficacy. Consequently, alternative therapies are needed.

### SUMMARY

Provided herein are methods, compositions, and uses relating to inhibitors of stem cell factor. For example, provided herein are antibodies targeting stem cell factor and methods for treating fibrotic and tissue remodeling diseases as well as for research and diagnostic uses.

In some embodiments, the compositions, methods, and uses herein provide therapies relating to inhibiting stem cell factor (SCF). Some embodiments provide an isolated antibody that targets SCF. In some embodiments, inhibiting SCF affects the activity of c-Kit. The compositions, methods, and uses provided herein find use in treating fibrotic diseases and maladies associated with tissue remodeling. Unlike some other therapies that produce undesirable side effects due to interfering with general intracellular signaling pathways, the embodiments provided herein eliminate or minimize such side effects by modulating the activity of SCF. Consequently, toxicity is minimized. Moreover, targeting an extracellular ligand removes the need to deliver a composition into a cell to interact with an intracellular target. In some embodiments, the compositions are delivered into the airway, thus providing an advantage over previous technologies that require oral administration and, as such, resulting in systemic bioavailability.

Provided herein are embodiments of methods for treating a fibrotic or tissue remodeling disease comprising administering a therapeutically effective amount of a stem cell factor inhibitor to a subject with or at risk for a fibrotic or tissue remodeling disease. For example, in some embodiments, provided herein are methods comprising providing an inhibitor of stem cell factor and administering a therapeutically effective amount of the inhibitor to a subject. In some embodiments the inhibitor is an isolated antibody (e.g., a monoclonal or polyclonal antibody) or an antigen-binding fragment thereof (e.g., Fab, Fab′, F(ab′)₂, and Fv fragments, etc.). In some embodiments the inhibitor is a small interfering RNA. In more specific embodiments, the antibody is a monoclonal antibody or a polyclonal antibody. Some embodiments provide that the antibody or antigen-binding fragment thereof specifically binds to stem cell factor. Some embodiments provide that the antibody or antigen-binding fragment thereof specifically binds to a peptide comprising amino acid sequence SEQ ID NO: 1 or SEQ ID NO: 8.

In some embodiments of the methods provided herein, the subject has a disease. Accordingly, some embodiments provide that administering the inhibitor prevents or reduces the severity of at least one sign or symptom of the disease. In some embodiments, the subject has an abnormal activity of stem cell factor or the subject has abnormal collagen production. In some embodiments, the subject has a disease including, but not limited to, fibrosis or a remodeling disease. In additional embodiments, the disease is a pulmonary disease. Some embodiments provide that a subject has a pulmonary disease including, but not limited to, idiopathic pulmonary fibrosis, chronic obstructive pulmonary disease, acute respiratory distress syndrome, cystic fibrosis, peribronchial fibrosis, hypersensitivity pneumonitis, or asthma. In addition, some embodiments provide that a subject has a disease including, but not limited to, sclerodoma, inflammation, liver cirrhosis, renal fibrosis, parenchymal fibrosis, endomyocardial fibrosis, mediatinal fibrosis, nodular subepidermal fibrosis, fibrous histiocytoma, fibrothorax, hepatic fibrosis, fibromyalgia, gingival fibrosis, or radiation-induced fibrosis.

While not limited in the mode of administration, in some embodiments of the method, the antibody is delivered into an airway of the subject, e.g., by intranasal administration.

In some embodiments, administering the inhibitor reduces the activity of a receptor. Some embodiments provide that administering the inhibitor reduces an interaction of stem cell factor with a receptor. In more specific embodiments, the receptor is a receptor tyrosine kinase, and in yet more specific embodiments, the receptor is c-Kit. Importantly, the methods are not limited in the location of the targeted receptor or the origin of stem cell factor. For example, in some embodiments the receptor is found on a hematopoietic progenitor cell, a melanocyte, a germ cell, an eosinophil, a lymphocyte, a fibroblast, a myofibroblast, or a mast cell. Additionally, in some embodiments, stem cell factor originates from a bone marrow cell, a liver cell, an epithelial cell, a smooth muscle cell, or a fibroblast. In some embodiments, administering the inhibitor to a subject results in a direct inhibition of fibroblast activation.

Some embodiments provide a composition comprising an isolated antibody (e.g., a monoclonal or a polyclonal antibody) or antigen-binding fragment thereof that specifically binds to stem cell factor (e.g., a protein or a peptide fragment thereof (e.g., an epitope)). For example, some embodiments provide a composition comprising an isolated antibody or antigen-binding fragment thereof that specifically binds to a peptide of amino acid sequence SEQ ID NO: 1 or SEQ ID NO: 8. Additional embodiments provide an antibody or antigen-binding fragment than binds to the SCF isoform b precursor (e.g., a protein or peptide fragment of the sequence available at GenBank accession number NP_000890 (SEQ ID NO: 4)), or a variant or modified form thereof, or to the SCF isoform a precursor (e.g., a protein or peptide fragment of the sequence available at GenBank accession number NP_003985 (SEQ ID NO: 6)), or a variant or modified form thereof. Some embodiments provide an antibody or antigen-binding fragment that binds to a protein or peptide, or variants or modified forms thereof, that is a translation product of the NCBI Reference Gene Sequence for SCF (e.g., accession number NG_012098 (SEQ ID NO: 7)) or variants or fragments thereof. Some embodiments provide an antibody or antigen-binding fragment that binds to a peptide comprising the first 11 amino acids of the mature form of SCF (e.g., EGICRNRVTNN (SEQ ID NO: 8)).

Some embodiments provide an antibody or antigen-binding fragment than binds to the translation product (e.g., a protein or peptide), or a variant or modified form thereof, of a nucleic acid encoding SCF, or a variant or a modified form thereof. For example, embodiments provide an antibody or antigen-binding fragment than binds to the translation product (e.g., a protein or peptide), or a variant or modified form thereof, of the nucleic acids having sequences comprising a sequence as defined by GenBank accession numbers NM_000899 (SEQ ID NO: 3), NM_003994 (SEQ ID NO: 5), and NG_012098 (SEQ ID NO: 7), or fragments or variants thereof (e.g., mutants, cDNAs, expression-optimized variants, operably linked to a regulatory element (e.g., promoter, enhancer, polymerase binding site, etc.), etc.). In some embodiments, the antibody or antigen-binding fragment binds to a protein or peptide, or a variant or modified form thereof, that is the translation product of a nucleotide sequence that encodes the peptide sequence EGICRNRVTNN (SEQ ID NO: 8). The peptides and proteins (and fragments and variants thereof) and the nucleic acids (and fragments and variants thereof) that encode the peptides and proteins (and fragments and variants thereof) are used in some embodiments to raise antibodies. Also contemplated are vectors, plasmids, expression constructs, cells, cell lines, hybridomas, and organisms used to produce the antibodies as provided herein.

Some embodiments provide a monoclonal antibody and some embodiments provide a humanized antibody. In some embodiments, the composition is used for a medicament or is used for the manufacture of a medicament. In some embodiments, the medicament is used to treat disease. Use of the composition as a medicament is not limited in the disease that can be treated. For example, in some embodiments, the disease is idiopathic pulmonary fibrosis, chronic obstructive pulmonary disease, acute respiratory distress syndrome, cystic fibrosis, peribronchial fibrosis, hypersensitivity pneumonitis, asthma, sclerodoma, inflammation, liver cirrhosis, renal fibrosis, parenchymal fibrosis, endomyocardial fibrosis, mediatinal fibrosis, nodular subepidermal fibrosis, fibrous histiocytoma, fibrothorax, hepatic fibrosis, fibromyalgia, gingival fibrosis, or radiation-induced fibrosis. In some embodiments, the composition is used to study disease in vitro or in a model system (e.g., in vivo).

Embodiments provide herein a method of preparing an antibody (e.g., a monoclonal antibody) targeting stem cell factor comprising the steps of providing a peptide comprising or consisting of an immunogenic portion of SCF (e.g., as provided by SEQ ID NO: 1 or 8), immunizing a host with the peptide, isolating an immune cell from the host, preparing a hybridoma using the immune cell, and isolating the antibody or antigen-binding fragment thereof. Some embodiments provide a method of preparing an antibody (e.g., a monoclonal antibody) targeting stem cell factor, wherein the antibody or antigen-binding fragment thereof specifically binds to stem cell factor (e.g., a protein or a peptide fragment thereof (e.g., an epitope)). For example, some embodiments provide a method of preparing an isolated antibody or antigen-binding fragment thereof that specifically binds to a peptide of amino acid sequence SEQ ID NO: 1. Additional embodiments provide a method of preparing an antibody or antigen-binding fragment than binds to the SCF isoform b precursor (e.g., a protein or peptide fragment of the sequence available at GenBank accession number NP_000890 (SEQ ID NO: 4)), or a variant or modified form thereof, or to the SCF isoform a precursor (e.g., a protein or peptide fragment of the sequence available at GenBank accession number NP_003985 (SEQ ID NO: 6)), or a variant or modified form thereof. Some embodiments provide a method of preparing an antibody or antigen-binding fragment that binds to a protein or peptide, or variants or modified forms thereof, that is a translation product of the NCBI Reference Gene Sequence for SCF (e.g., accession number NG_012098 (SEQ ID NO: 7)) or variants or fragments thereof. Some embodiments provide a method of preparing an antibody or antigen-binding fragment that binds to a peptide comprising the first 11 amino acids of the mature form of SCF (e.g., EGICRNRVTNN (SEQ ID NO: 8)).

Some embodiments provide a method of preparing an antibody or antigen-binding fragment than binds to the translation product (e.g., a protein or peptide), or a variant or modified form thereof, of a nucleic acid encoding SCF, or a variant or a modified form thereof. For example, embodiments provide a method of preparing an antibody or antigen-binding fragment than binds to the translation product (e.g., a protein or peptide), or a variant or modified form thereof, of the nucleic acids having sequences comprising a sequence as defined by GenBank accession numbers NM_000899 (SEQ ID NO: 3), NM_003994 (SEQ ID NO: 5), and NG_012098 (SEQ ID NO: 7), or fragments or variants thereof (e.g., mutants, cDNAs, expression-optimized variants, operably linked to a regulatory element (e.g., promoter, enhancer, polymerase binding site, etc.), etc.). In some embodiments, the antibody or antigen-binding fragment binds to a protein or peptide, or a variant or modified form thereof, that is the translation product of a nucleotide sequence that encodes the peptide sequence EGICRNRVTNN (SEQ ID NO: 8). The peptides, proteins, and fragments and variants thereof; and nucleic acids, and fragments and variants thereof, that encode the peptides, proteins, and fragments and variants thereof, find use in some embodiments in a method of preparing antibodies as provided by the technology provided. Also contemplated are methods of producing vectors, plasmids, expression constructs, cells, cell lines, hybridomas, and organisms that find use in producing the antibodies as provided herein.

Some embodiments provide a method comprising the steps of providing an inhibitor of stem cell factor and administering the inhibitor to a cell or tissue.

In addition, some embodiments provide a kit comprising a composition comprising an isolated antibody or antigen-binding fragment thereof that specifically binds to stem cell factor, a means for administering the composition to a subject, and/or instructions for use.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 shows a series of plots demonstrating that inhibiting SCF with an antibody reduces the expression of tissue remodeling mediators. FIG. 1A shows a plot demonstrating that an anti-SCF antibody reduces the amount of hydroxyproline in bleomycin treated lung;

FIG. 1B shows a plot demonstrating that an anti-SCF antibody reduces the amount of IL-25 mRNA; FIG. 1C shows a plot demonstrating that an anti-SCF antibody reduces the amount of IL-13 mRNA; FIG. 1D shows a plot demonstrating that an anti-SCF antibody reduces the amount of soluble SCF present in plasma. FIG. 1E shows a plot demonstrating that an anti-SCF antibody reduces the amount of IL-25 receptor.

FIG. 2 shows a plot demonstrating that IL-4 stimulates c-kit expression in human fibroblasts.

FIG. 3 shows an amino acid sequence and the corresponding nucleotide sequence of an immunogenic peptide used to produce antibodies specific for SCF.

FIG. 4 shows a plot demonstrating that a monoclonal antibody specific for SCF inhibits the activation of HMC-1 cells for MCP-1 production.

FIG. 5 shows a plot demonstrating that a lower amount of hydroxyproline is detected in a mouse deficient in SCF production after bleomycin injury.

### DETAILED DESCRIPTION

Provided herein are methods, compositions, and uses relating to inhibitors of stem cell factor. For example, provided herein are antibodies targeting stem cell factor, methods of producing antibodies targeting stem cell factor, and methods for treating fibrotic and tissue remodeling diseases as well as for research and diagnostic uses. In some embodiments, the compositions, methods, and uses herein provide therapies relating to inhibiting stem cell factor (SCF). Some embodiments provide an isolated antibody that targets SCF. In some embodiments, inhibiting SCF affects the activity of c-Kit. The compositions, methods, and uses provided herein find use in treating fibrotic diseases and maladies associated with tissue remodeling.

### Definitions

To facilitate an understanding of embodiments of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein.

Where the term “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “nascent” when used in reference to a protein refers to a newly synthesized protein, which has not been subject to post-translational modifications, which includes but is not limited to glycosylation and polypeptide shortening. The term “mature” when used in reference to a protein refers to a protein which has been subject to post-translational processing and/or which is in a cellular location (such as within a membrane or a multi-molecular complex) from which it can perform a particular function which it could not if it were not in the location.

The term “portion” when used in reference to a protein (as in “a portion of a given protein”) refers to fragments of that protein. The fragments may range in size from four amino acid residues to the entire amino sequence minus one amino acid (for example, the range in size includes 4, 5, 6, 7, 8, 9, 10, or 11 . . . amino acids up to the entire amino acid sequence minus one amino acid).

The term “homolog” or “homologous” when used in reference to a polypeptide refers to a high degree of sequence identity between two polypeptides, or to a high degree of similarity between the three-dimensional structure or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include the transmembrane domains, and the glycosylation sites.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The terms “oligonucleotide” or “polynucleotide” or “nucleotide” or “nucleic acid” refer to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The terms “an oligonucleotide having a nucleotide sequence encoding a gene” or “a nucleic acid sequence encoding” a specified polypeptide refer to a nucleic acid sequence comprising the coding region of a gene or in other words the nucleic acid sequence which encodes a gene product. The coding region may be present in either a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

The terms “complementary” and “complementarity” refer to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is frequently that gene which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “allele” refers to different variations in a gene; the variations include but are not limited to variants and mutants, polymorphic loci and single nucleotide polymorphic loci, frameshift and splice mutations. An allele may occur naturally in a population, or it might arise during the lifetime of any particular individual of the population.

Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; typically, one sequence is a reference sequence.

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex which is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (e.g., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

The term “purified” refers to molecules, either nucleic or amino acid sequences, that are removed from their natural environment, isolated or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the term “purified” or “to purify” also refer to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution. Compositions comprising polynucleotide sequences or fragments thereof may be employed as hybridization probes. In some embodiments, polynucleotide sequences are employed in an aqueous solution containing salts (e.g., NaCl), detergents (e.g., SDS), and other components (e.g., Denhardt's solution, dry milk, salmon sperm DNA, etc.).

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “antibody” is used in its broadest sense to refer to whole antibodies, monoclonal antibodies (including human, humanized, or chimeric antibodies), polyclonal antibodies, and antibody fragments that can bind antigen (e.g., Fab′, F′ (ab) 2, Fv, single chain antibodies), comprising complementarity determining regions (CDRs) of the foregoing as long as they exhibit the desired biological activity.

As used herein, “antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8 (10): 1057-1062 (1995)); single-chain antibody molecules; and multispecific antibodies formed from antibody fragments.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

As used herein, “active” or “activity” refers to native or naturally occurring biological and/or immunological activity.

As used herein the term, “in vitro” refers to an artificial environment and to processes or reactions that occur within an artificial environment. In vitro environments may include, but are not limited to, test tubes and cell cultures. The term “in vivo” refers to the natural environment (e.g., an animal or a cell) and to processes or reactions that occur within a natural environment.

As used herein, “inhibitor” refers to a molecule which eliminates, minimizes, or decreases the activity, e.g., the biological, enzymatic, chemical, or immunological activity, of a target.

As used herein the term “disease” refers to a deviation from the condition regarded as normal or average for members of a species, and which is detrimental to an affected individual under conditions that are not inimical to the majority of individuals of that species (e.g., diarrhea, nausea, fever, pain, inflammation, etc.).

As used herein, the term “administration” refers to the act of giving a drug, prodrug, antibody, or other agent, or therapeutic treatment to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like. “Coadministration” refers to administration of more than one chemical agent or therapeutic treatment (e.g., radiation therapy) to a physiological system (e.g., a subject or in vivo, in vitro, or ex vivo cells, tissues, and organs). As used herein, administration “in combination with” one or more further therapeutic agents includes simultaneous (concurrent) and consecutive administration in any order. “Coadministration” of therapeutic treatments may be concurrent, or in any temporal order or physical combination.

As used herein, the term “treating” includes reducing or alleviating at least one adverse effect or symptom of a disease or disorder through introducing in any way a therapeutic composition of the present technology into or onto the body of a subject. “Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

As used herein, “therapeutically effective dose” refers to an amount of a therapeutic agent sufficient to bring about a beneficial or desired clinical effect. Said dose can be administered in one or more administrations. However, the precise determination of what would be considered an effective dose may be based on factors individual to each patient, including, but not limited to, the patient's age, size, type or extent of disease, stage of the disease, route of administration, the type or extent of supplemental therapy used, ongoing disease process, and type of treatment desired (e.g., aggressive vs. conventional treatment).

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the term “pharmaceutical composition” refers to the combination of an active agent with, as desired, a carrier, inert or active, making the composition especially suitable for diagnostic or therapeutic use in vitro, in vivo, or ex vivo.

As used herein, the terms “pharmaceutically acceptable” or “pharmacologically acceptable” refer to compositions that do not substantially produce adverse reactions, e.g., toxic, allergic, or immunological reactions, when administered to a subject.

As used herein, “carriers” include pharmaceutically acceptable carriers, excipients, or stabilizers which are nontoxic to the cell or mammal being exposed thereto at the dosages and concentrations employed. Often the physiologically acceptable carrier is an aqueous pH-buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

As used herein, the terms “patient” or “subject” refer to organisms to be treated by the compositions of the present technology or to be subject to various tests provided by the technology. The term “subject” includes animals, preferably mammals, including humans. In a preferred embodiment, the subject is a primate. In an even more preferred embodiment, the subject is a human.

As used herein, the term “sample” is used in its broadest sense. In one sense it can refer to animal cells or tissues. In another sense, it is meant to include a specimen or culture obtained from any source, such as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present technology.

### Embodiments of the Technology

Although the disclosure herein refers to certain illustrated embodiments, it is to be understood that these embodiments are presented by way of example and not by way of limitation.

### 1. Inhibitors of SCF

Stem cell factor (SCF) is a ligand that is specific for the c-Kit receptor kinase. Binding of SCF to c-Kit causes dimerization of c-Kit and activation of its kinase activity, which is important for hemopoiesis, melanogenesis, and fertility. Through c-Kit, SCF acts to promote cell survival, proliferation, differentiation, adhesion, and functional activation. Aberrant activation of c-Kit can result in disease, including fibrosis and tissue remodeling defects. In particular, there are multiple pulmonary diseases with known remodeling defects as well as other chronic tissue remodeling diseases affecting other organs and tissues. Specific examples of diseases involving fibrosis or tissue remodeling defects are idiopathic pulmonary fibrosis, chronic obstructive pulmonary disease, acute respiratory distress syndrome, cystic fibrosis, peribronchial fibrosis, hypersensitivity pneumonitis, asthma, sclerodoma, inflammation, liver cirrhosis, renal fibrosis, parenchymal fibrosis, endomyocardial fibrosis, mediatinal fibrosis, nodular subepidermal fibrosis, fibrous histiocytoma, fibrothorax, hepatic fibrosis, fibromyalgia, gingival fibrosis, and radiation-induced fibrosis.

Accordingly, interfering with the interaction between SCF and c-Kit can be used to treat or study diseases involving aberrant activation of c-Kit that causes fibrosis and tissue remodeling defects. The c-Kit receptor is found on hematopoietic progenitor cells, melanocytes, germ cells, eosinophils, lymphocytes, and mast cells. Thus, preventing SCF interaction with c-Kit can alter the activation of several disease-associated cell populations that have been implicated in fibrosis and tissue remodeling disease phenotypes.

Additionally, SCF induces key mediators in the fibrotic response, IL-25 and IL-13. Data suggest that IL-25 can drive IL-13 expression in a T-cell and antigen-independent manner. Therefore, these processes can progress without an antigen-specific response and consequently chronically perpetuate remodeling and fibrotic disease. It is contemplated that a complex cascade is established in which SCF induces IL-25, which in turn induces production of IL-13, myofibroblast differentiation, and collagen production. IL-4 has also been identified as a fibrosis-associated cytokine.

### 2. Antibodies

In some embodiments, inhibiting the ability of SCF to interact with c-Kit is accomplished by means of an antibody that recognizes SCF. The antibody can be a monoclonal antibody or a polyclonal antibody, and may be, for example, a human, humanized, or chimeric antibody. Monoclonal antibodies against target antigens are produced by a variety of techniques including conventional monoclonal antibody methodologies such as the somatic cell hybridization techniques of Köhler and Milstein (Nature, 256:495 (1975)). Although in some embodiments, somatic cell hybridization procedures are preferred, other techniques for producing monoclonal antibodies are contemplated as well (e.g., viral or oncogenic transformation of B lymphocytes).

It is contemplated that antibodies against SCF find use in the experimental, diagnostic, and therapeutic methods described herein. In certain embodiments, the antibodies provided herein are used to detect the expression of SCF in biological samples. For example, a sample comprising a tissue biopsy can be sectioned and protein detected using, for example, immunofluorescence or immunohistochemistry. Alternatively, individual cells from a sample can be isolated, and protein expression detected on fixed or live cells by FACS analysis. Furthermore, the antibodies can be used on protein arrays to detect expression of SCF. In other embodiments, the antibodies provided herein are used to decrease the activity of cells expressing c-Kit by inhibiting SCF either in an in vitro cell-based assay or in an in vivo animal model. In some embodiments, antibodies are used to treat a human patient by administering a therapeutically effective amount of an antibody against SCF.

For the production of antibodies, various host animals can be immunized by injection with the peptide corresponding to the desired epitope (e.g., a fragment of SCF, e.g., a fragment comprising the sequence provided by SEQ ID NO: 1 or 8 or immunogenic portions thereof) including, but not limited to, rabbits, mice, rats, sheep, goats, etc. Antibodies to SCF can be raised by immunizing (e.g., by injection) with an antigen comprising a peptide, a portion, or the full protein of the SCF isoform b precursor (e.g., a protein or peptide fragment of the sequence available at GenBank accession number NP_000890 (SEQ ID NO: 4)), or a variant or modified version thereof, or a peptide, a portion, or the full protein of the SCF isoform a precursor (e.g., a protein or peptide fragment of the sequence available at GenBank accession number NP_003985 (SEQ ID NO: 6)), or a variant or modified version thereof. Antibodies can also be raised by immunization with a translation product of the NCBI Reference Gene Sequence for SCF (e.g., accession number NG_012098 (SEQ ID NO: 7)) or variants or fragments thereof.

In some embodiments, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

Polyclonal antibodies can be prepared by any known method. Polyclonal antibodies can be raised by immunizing an animal (e.g., a rabbit, rat, mouse, donkey, etc) by multiple subcutaneous or intraperitoneal injections of the relevant antigen (a purified peptide fragment, full-length recombinant protein, fusion protein, etc.) optionally conjugated to KLH, serum albumin, etc., diluted in sterile saline, and combined with an adjuvant to form a stable emulsion. The polyclonal antibody is then recovered from blood, ascites, and the like, of an animal so immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc.

For preparation of monoclonal antibodies, any technique that provides for the production of antibody molecules by continuous cell lines in culture may be used (see e.g., Harlow and Lane, Antibodies: A Laboratory Manual, Cold Spring Harbor Laboratory Press, Cold Spring Harbor, NY). These include, but are not limited to, the hybridoma technique originally developed by Köhler and Milstein and the trioma technique, the human B-cell hybridoma technique (See, e.g., Kozbor et al., Immunol. Today, 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

In some embodiments provided herein, the antibodies are prepared from a hybridoma. Using the hybridoma method, a mouse, hamster, or other appropriate host animal, is immunized as described above to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay such as radioimmunoassay (RIA) or enzyme-linked immunosorbent assay (ELISA) can then be propagated in vitro (e.g., in culture) using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

The preferred animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known. Embodiments of the technology herein provide antibodies (e.g., monoclonal antibodies) produced from a hybridoma prepared by immunizing mice with a peptide that is a portion or fragment of the SCF protein. For example, some embodiments provide an antibody or antigen-binding fragment than binds to SCF by immunizing with, e.g., a protein or peptide fragment of the sequence available at GenBank accession number NP_000890 (SEQ ID NO: 4)), or a variant or modified version thereof, or by immunizing with, e.g., a protein or peptide fragment of the sequence available at GenBank accession number NP_003985 (SEQ ID NO: 6)), or a variant or modified version thereof. Some embodiments provide an antibody or antigen-binding fragment that binds to a protein or peptide, or variants or modified versions thereof, that is a translation product of the NCBI Reference Gene Sequence for SCF (e.g., accession number NG_012098 (SEQ ID NO: 7)) or variants or fragments thereof.

For example, embodiments of the technology herein provide monoclonal antibodies produced from a hybridoma prepared by immunizing mice with a peptide of amino acid sequence SEQ ID NO: 1 or 8. Also contemplated are methods and compositions related to antibodies prepared using a variant of SEQ ID NO: 1 or 8 comprising one or more substitutions, deletions, insertions, or other changes, as long as said variant produces an antibody specific for SCF. Producing polypeptides of SEQ ID NO: 1 or 8 and similar sequences thereto can be accomplished according to various techniques well known in the art. For example, a polypeptide of SEQ ID NO: 1 or 8 or a variant thereof can be produced using a bacterial expression system and a nucleic acid encoding a polypeptide of SEQ ID NO: 1 or 8 or a variant thereof. As an example, a polypeptide according to SEQ ID NO: 1 can be produced using the nucleotide sequence according to SEQ ID NO: 2.

Moreover, human monoclonal antibodies directed against human proteins can be generated using transgenic mice carrying the complete human immune system rather than the mouse system. Splenocytes from the transgenic mice are immunized with the antigen of interest, which are used to produce hybridomas that secrete human monoclonal antibodies with specific affinities for epitopes from a human protein.

Monoclonal antibodies can also be generated by other methods known to those skilled in the art of recombinant DNA technology. For instance, combinatorial antibody display has can be utilized to produce monoclonal antibodies (see, e.g., Sastry et al., Proc. Nat. Acad. Sci. USA, 86:5728 (1989); Huse et al., Science, 246:1275 (1989); Orlandi et al., Proc. Nat. Acad. Sci. USA, 86:3833 (1989)). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primers to a conserved 3′ region can be used to amplify and isolate the heavy and light chain variable regions from a number of murine antibodies (see. e.g., Larrick et al., Biotechniques, 11:152 (1991)). A similar strategy can also been used to amplify human heavy and light chain variable regions from human antibodies (see, e.g., Larrick et al., Methods: Companion to Methods in Enzymology, 2:106 (1991)).

Alternatively, monoclonal antibodies can also be made using recombinant DNA methods as described in U.S. Pat. No. 4,816,567. The polynucleotides encoding a monoclonal antibody are isolated (e.g., from mature B-cells or hybridoma cells), by, e.g., RT-PCR using oligonucleotide primers that specifically amplify the genes encoding the heavy and light chains of the antibody, and their sequences are determined using conventional procedures. The isolated polynucleotides encoding the heavy and light chains are then cloned into suitable expression vectors, which, when transfected into host cells such as E. coli cells, simian COS cells, Chinese hamster ovary (CHO) cells, or myeloma cells that do not otherwise produce immunoglobulin protein, cause monoclonal antibodies to be generated by the host cells. Also, recombinant monoclonal antibodies or fragments thereof of the desired species can be isolated from phage display libraries as described (McCafferty et al., 1990, Nature, 348:552-554; Clackson et al., 1991, Nature, 352:624-628; and Marks et al., 1991, J. Mol. Biol., 222:581-597).

The polynucleotide encoding a monoclonal antibody can further be modified in a number of different manners using recombinant DNA technology to generate alternative antibodies. In one embodiment, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity, affinity, etc. of a monoclonal antibody.

For example, also contemplated are chimeric mouse-human monoclonal antibodies, which can be produced by recombinant DNA techniques known in the art. For example, a gene encoding the constant region of a murine (or other species) monoclonal antibody molecule is digested with restriction enzymes to remove the region encoding the murine constant region, and the equivalent portion of a gene encoding a human constant region is substituted (see, e.g., Robinson et al., PCT/US86/02269; European Patent Application 184,187; European Patent Application 171,496; European Patent Application 173,494; WO 86/01533; U.S. Pat. No. 4,816,567; European Patent Application 125,023 (each of which is herein incorporated by reference in its entirety); Better et al., Science, 240:1041-1043 (1988); Liu et al., Proc. Nat. Acad. Sci. USA, 84:3439-3443 (1987); Liu et al., J. Immunol., 139:3521-3526 (1987); Sun et al., Proc. Nat. Acad. Sci. USA, 84:214-218 (1987); Nishimura et al., Canc. Res., 47:999-1005 (1987); Wood et al., Nature, 314:446-449 (1985); and Shaw et al., J. Natl. Cancer Inst., 80:1553-1559 (1988)).

The chimeric antibody can be further humanized by replacing sequences of the variable region that are not directly involved in antigen binding with equivalent sequences from human variable regions. General reviews of humanized chimeric antibodies are provided by S. L. Morrison, Science, 229:1202-1207 (1985) and by Oi et al., Bio Techniques, 4:214 (1986). Those methods include isolating, manipulating, and expressing the nucleic acid sequences that encode all or part of immunoglobulin variable regions from at least one of a heavy or light chain. Sources of such nucleic acid are well known to those skilled in the art. The recombinant DNA encoding the chimeric antibody, or fragment thereof, can then be cloned into an appropriate expression vector.

Suitable humanized antibodies can alternatively be produced by CDR substitution (see, e.g., U.S. Pat. No. 5,225,539; Jones et al., Nature, 321:552-525 (1986); Verhoeyan et al., Science, 239:1534 (1988); and Beidler et al., J. Immunol., 141:4053 (1988)). All of the CDRs of a particular human antibody may be replaced with at least a portion of a non-human CDR or only some of the CDRs may be replaced with non-human CDRs. It is only necessary to replace the number of CDRs important for binding of the humanized antibody to the Fc receptor.

An antibody can be humanized by any method that is capable of replacing at least a portion of a CDR of a human antibody with a CDR derived from a non-human antibody. The human CDRs may be replaced with non-human CDRs using oligonucleotide site-directed mutagenesis.

Also contemplated are chimeric and humanized antibodies in which specific amino acids have been substituted, deleted, or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances.

In certain embodiments provided herein, it is desirable to use an antibody fragment. Various techniques are known for the production of antibody fragments. Traditionally, these fragments are derived via proteolytic digestion of intact antibodies (for example Morimoto et al., 1993, Journal of Biochemical and Biophysical Methods 24:107-117 and Brennan et al., 1985, Science, 229:81). For example, papain digestion of antibodies produces two identical antigen-binding fragments, called Fab fragments, each with a single antigen-binding site, and a residual Fc fragment. Pepsin treatment yields an F(ab′)₂ fragment that has two antigen-combining sites and is still capable of cross-linking antigen.

However, these fragments are now typically produced directly by recombinant host cells as described above. Thus Fab, Fv, and scFv antibody fragments can all be expressed in and secreted from E. coli or other host cells, thus allowing the production of large amounts of these fragments. Alternatively, such antibody fragments can be isolated from the antibody phage libraries discussed above. The antibody fragment can also be linear antibodies as described in U.S. Pat. No. 5,641,870, for example, and can be monospecific or bispecific. Other techniques for the production of antibody fragments will be apparent to the skilled practitioner.

Fv is the minimum antibody fragment which contains a complete antigen-recognition and antigen-binding site. This region consists of a dimer of one heavy-chain and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. F(ab′)₂ antibody fragments originally were produced as pairs of Fab′ fragments which have hinge cysteines between them. Other chemical couplings of antibody fragments are also known to the skilled artisan.

The technology herein provided also contemplates modifying an antibody to increase its serum half-life. This can be achieved, for example, by incorporating a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

The technology embraces variants and equivalents which are substantially homologous to the chimeric, humanized, and human antibodies, or antibody fragments thereof, provided herein. These can contain, for example, conservative substitution mutations, i.e. the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid. What is intended by a conservative amino acid substitution is well known in the art.

An additional embodiment utilizes the techniques known in the art for the construction of Fab expression libraries (Huse et al., Science, 246:1275-1281 (1989)) to allow rapid and easy identification of monoclonal Fab fragments with the desired specificity.

Also, this technology encompasses bispecific antibodies that specifically recognize SCF. Bispecific antibodies are antibodies that are capable of specifically recognizing and binding at least two different epitopes. Bispecific antibodies can be intact antibodies or antibody fragments. Techniques for making bispecific antibodies are common in the art (Millstein et al., 1983, Nature 305:537-539; Brennan et al., 1985, Science 229:81; Suresh et al, 1986, Methods in Enzymol. 121:120; Traunecker et al., 1991, EMBO J. 10:3655-3659; Shalaby et al., 1992, J. Exp. Med. 175:217-225; Kostelny et al., 1992, J. Immunol. 148:1547-1553; Gruber et al., 1994, J. Immunol. 152:5368; and U.S. Pat. No. 5,731,168).

Techniques described for the production of single chain antibodies (U.S. Pat. No. 4,946,778; herein incorporated by reference) can be adapted to produce specific single chain antibodies as desired. Single-chain Fv antibody fragments comprise the V_{H} and V_{L} domains of an antibody, wherein these domains are present in a single polypeptide chain. Preferably, the Fv polypeptide further comprises a polypeptide linker between the V_{H} and V_{L} domains that enables the single-chain Fv antibody fragments to form the desired structure for antigen binding. For a review of single-chain Fv antibody fragments, see Pluckthun in The Pharmacology of Monoclonal Antibodies, vol. 113, Rosenburg and Moore eds., Springer-Verlag, New York, pp. 269-315 (1994).

### 3. Other SCF Inhibitors

It is also contemplated that inhibiting SCF can be accomplished by a variety of other types of inhibitors. For example, in some embodiments a small interfering RNA (siRNA) can be designed to target and degrade SCF mRNA. siRNAs are double-stranded RNA molecules of 20-25 nucleotides in length. While not limited in their features, typically an siRNA is 21 nucleotides long and has 2-nt 3′ overhangs on both ends. Each strand has a 5′ phosphate group and a 3′ hydroxyl group. In vivo, this structure is the result of processing by dicer, an enzyme that converts either long dsRNAs or small hairpin RNAs into siRNAs. However, siRNAs can also be synthesized and exogenously introduced into cells to bring about the specific knockdown of a gene of interest. Essentially any gene of which the sequence is known can be targeted based on sequence complementarity with an appropriately tailored siRNA. For example, those of ordinary skill in the art can synthesize an siRNA (see, e.g., Elbashir, et al., Nature 411:494 (2001); Elbashir, et al. Genes Dev 15:188 (2001); Tuschl T, et al., Genes Dev 13:3191 (1999)).

In some embodiments, RNAi is utilized to inhibit SCF. RNAi represents an evolutionarily conserved cellular defense for controlling the expression of foreign genes in most eukaryotes, including humans. RNAi is typically triggered by double-stranded RNA (dsRNA) and causes sequence-specific degradation of single-stranded target RNAs (e.g., an mRNA). The mediators of mRNA degradation are small interfering RNAs (siRNAs), which are normally produced from long dsRNA by enzymatic cleavage in the cell. siRNAs are generally approximately twenty-one nucleotides in length (e.g. 21-23 nucleotides in length) and have a base-paired structure characterized by two nucleotide 3′ overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, an RNase III enzyme (e.g., Dicer) converts the longer dsRNA into 21-23 nt double-stranded siRNA fragments. In some embodiments, RNAi oligonucleotides are designed to target the junction region of fusion proteins. Chemically synthesized siRNAs have become powerful reagents for genome-wide analysis of mammalian gene function in cultured somatic cells. Beyond their value for validation of gene function, siRNAs also hold great potential as gene-specific therapeutic agents (see, e.g., Tuschl and Borkhardt, Molecular Intervent. 2002; 2 (3): 158-67, herein incorporated by reference).

The transfection of siRNAs into animal cells results in the potent, long-lasting post-transcriptional silencing of specific genes (Caplen et al, Proc Natl Acad Sci U.S.A. 2001; 98:9742-47; Elbashir et al., Nature. 2001; 411:4 94-98; Elbashir et al., Genes Dev. 2001; 15:188-200; and Elbashir et al., EMBO J. 2001; 20:6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA and their protein products, frequently to undetectable levels. The silencing effect can last several months, and is extraordinarily specific—a one-nucleotide mismatch between the target RNA and the central region of the siRNA is frequently sufficient to prevent silencing (Brummelkamp et al, Science 2002; 296:550-53; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278:15991-97; herein incorporated by reference) describe the use of a type of DNA array called a scanning array to find accessible sites in mRNAs for designing effective siRNAs. These arrays comprise oligonucleotides ranging in size from monomers to a certain maximum, usually Co-mers, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, e.g., to retain efficacy and target specificity (Sohail et al, Nucleic Acids Res., 2001; 29 (10): 2041-45). Additional methods and concerns for selecting siRNAs are described, for example, in WO 05054270, WO05038054A1, WO03070966A2, J Mol Biol. 2005-5-13; 348 (4): 883-93, J Mol Biol. 2005 May 13; 348 (4): 871-81, and Nucleic Acids Res. 2003-8-1; 31 (15): 4417-24, each of which is herein incorporated by reference in its entirety. In addition, software (e.g., the MWG online siMAX siRNA design tool) is commercially or publicly available for use in the selection and design of siRNAs and RNAi reagents.

In some embodiments, the present invention utilizes siRNA including blunt ends (See e.g., US20080200420, herein incorporated by reference in its entirety), overhangs (See e.g., US20080269147A1, herein incorporated by reference in its entirety), locked nucleic acids (See e.g., WO2008/006369, WO2008/043753, and WO2008/051306, each of which is herein incorporated by reference in its entirety). In some embodiments, siRNAs are delivered via gene expression or using bacteria (See e.g., Xiang et al., Nature 24:6 (2006) and WO06066048, each of which is herein incorporated by reference in its entirety).

In other embodiments, shRNA techniques (See e.g., 20080025958, herein incorporated by reference in its enterety) are utilized. A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA polymerase III.

The present invention also includes pharmaceutical compositions and formulations that include the RNAi compounds of the present invention as described below.

SCF exists in both transmembrane and soluble forms. Upon cleavage of the SCF soluble domain from the transmembrane form, SCF is released from the cell surface to function as the ligand of c-Kit. Thus, it is contemplated that SCF activity can be altered by inhibiting the release of soluble SCF from the membrane-bound form, for example, by inhibiting or otherwise reducing the activity of a protease that cleaves the soluble domain from the membrane-bound form.

In addition, it is contemplated that SCF can be inhibited by chemicals (e.g., a small molecule, e.g., a pharmacological agent) or other biological agents that bind or modify SCF. For example, one of ordinary skill in the art can design and produce RNA aptamers or other nucleic acids that specifically recognize and bind to SCF, for instance by using SELEX or other in vitro evolution methods known in the art. Furthermore, SCF activity can be inhibited by specifically degrading SCF or inducing an altered conformation of SCF such that it is less effective in interacting with c-Kit. In some embodiments, the SCF inhibitor is a “designed ankyrin repeat protein” (DARPin) (see, e.g., Stumpp M T & Amstutz P, “DARPins: a true alternative to antibodies”, Curr Opin Drug Discov Devel 2007, 10 (2): 153-59, incorporated herein in its entirety for all purposes). In some embodiments, SCF is inhibited by a small molecule, e.g., a small molecule that binds to SCF and blocks its function (e.g., inhibits its binding and/or other interaction (e.g., an activating interaction) with the c-Kit receptor).

It is contemplated that altering SCF activity can be effected by inhibiting the expression of SCF, for instance, by inhibiting the transcription of SCF, by inhibiting the translation of SCF, by inhibiting the processing of the SCF mRNA, by inhibiting the processing of the SCF polypeptide, by inhibiting the folding of the SCF polypeptide, by inhibiting trafficking of SCF within a cell, or by inhibiting the insertion of SCF into the plasma membrane. SCF activity can be altered by changes in chromatin structure or other means of epigenetic regulation of SCF (e.g., changes in DNA methylation). Also, SCF activity may be altered by specifically sequestering SCF in a vesicle or other cellular compartment that hinders its action upon c-Kit.

### 4. Therapies Using Inhibitors of SCF

Inhibiting SCF finds use in therapies to treat disease. Accordingly, provided herein are therapies comprising inhibiting SCF to benefit individuals suffering from disease. In particular, as shown herein, disease states involving fibrosis and tissue remodeling demonstrate aberrant SCF activity. For example, fibroblasts isolated from diseased individuals with fibrotic or tissue remodeling phenotypes directly respond to SCF, which results in the generation of a more severe phenotype that includes increased collagen production. As such, as shown herein, inhibiting SCF can significantly affect the generation of severe disease consequences including inflammation and remodeling of target tissue. Also contemplated are therapies targeting SCF during the generation of fibrosis associated with acute and chronic disorders that have either a dynamic disease course or a more predictable disease course. Indications that can benefit from therapy inhibiting SCF include, but are not limited to, idiopathic pulmonary fibrosis, chronic obstructive pulmonary disease, acute respiratory distress syndrome, cystic fibrosis, peribronchial fibrosis, hypersensitivity pneumonitis, asthma, sclerodoma, inflammation, liver cirrhosis, renal fibrosis, parenchymal fibrosis, endomyocardial fibrosis, mediatinal fibrosis, nodular subepidermal fibrosis, fibrous histiocytoma, fibrothorax, hepatic fibrosis, fibromyalgia, gingival fibrosis, and radiation-induced fibrosis.

Importantly, therapies targeting SCF reduce or eliminate toxic effects associated with other similar therapies, for example those targeting c-Kit. These undesirable toxic effects are associated with targeting an intracellular, rather than extracellular, target, and the more widespread and general changes in cell signaling that result. While the therapies are not limited in their route of administration, embodiments of the technology provided herein deliver the SCF inhibitor via the airway by intranasal administration. Such administration allows direct delivery of the therapeutic agent to target tissues in pulmonary diseases involving fibrosis and tissue remodeling, rather than relying on systemic delivery via an orally administered composition.

In certain embodiments, a physiologically appropriate solution containing an effective concentration of an antibody specific for SCF can be administered topically, intraocularly, parenterally, orally, intranasally, intravenously, intramuscularly, subcutaneously, or by any other effective means. In particular, the antibody may delivered into an airway of a subject by intranasal administration. Alternatively, a tissue can receive a physiologically appropriate composition (e.g., a solution such as a saline or phosphate buffer, a suspension, or an emulsion, which is sterile) containing an effective concentration of an antibody specific for SCF via direct injection with a needle or via a catheter or other delivery tube. Any effective imaging device such as X-ray, sonogram, or fiber-optic visualization system may be used to locate the target tissue and guide the admistration. In another alternative, a physiologically appropriate solution containing an effective concentration of an antibody specific for SCF can be administered systemically into the blood circulation to treat tissue that cannot be directly reached or anatomically isolated. Such manipulations have in common the goal of placing an effective concentration of an antibody specific for SCF in sufficient contact with the target tissue to permit the antibody specific for SCF to contact the tissue.

With respect to administration of a SCF inhibitor (e.g., an antibody specific for SCF) to a subject, it is contemplated that the SCF inhibitor be administered in a pharmaceutically effective amount. One of ordinary skill recognizes that a pharmaceutically effective amount varies depending on the therapeutic agent used, the subject's age, condition, and sex, and on the extent of the disease in the subject. Generally, the dosage should not be so large as to cause adverse side effects, such as hyperviscosity syndromes, pulmonary edema, congestive heart failure, and the like. The dosage can also be adjusted by the individual physician or veterinarian to achieve the desired therapeutic goal.

As used herein, the actual amount encompassed by the term “pharmaceutically effective amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication, and other factors that those skilled in the art will recognize.

In some embodiments, a SCF inhibitor (e.g., an antibody specific for SCF) according to the technology provided herein is administered in a pharmaceutically effective amount. In some embodiments, a SCF inhibitor (e.g., an antibody specific for SCF) is administered in a therapeutically effective dose. The dosage amount and frequency are selected to create an effective level of the SCF inhibitor without substantially harmful effects. When administered, the dosage of a SCF inhibitor (e.g., an antibody specific for SCF) will generally range from 0.001 to 10,000 mg/kg/day or dose (e.g., 0.01 to 1000 mg/kg/day or dose; 0.1 to 100 mg/kg/day or dose).

Pharmaceutical compositions preferably comprise one or more compounds of the present invention associated with one or more pharmaceutically acceptable carriers, diluents, or excipients. Pharmaceutically acceptable carriers are known in the art such as those described in, for example, Remingtons Pharmaceutical Sciences, Mack Publishing Co. (A. R. Gennaro ed., 1985).

In some embodiments, a single dose of a SCF inhibitor (e.g., an antibody specific for SCF) according to the technology provided herein is administered to a subject. In other embodiments, multiple doses are administered over two or more time points, separated by hours, days, weeks, etc. In some embodiments, compounds are administered over a long period of time (e.g., chronically), for example, for a period of months or years (e.g., 1, 2, 3, 4, 5, 6, 7, 8, 9, 10, 11, 12, or more months or years; e.g., for the lifetime of the subject). In such embodiments, compounds may be taken on a regular scheduled basis (e.g., daily, weekly, etc.) for the duration of the extended period.

In some embodiments, a SCF inhibitor (e.g., an antibody specific for SCF) according to the technology provided herein is co-administered with another compound or more than one other compound (e.g., 2 or 3 or more other compounds).

### 5. Kits

Some embodiments provide herein kits for the treatment of a subject. In some embodiments, the kits include an inhibitor of SCF and appropriate solutions and buffers. Embodiments include all controls and instructions for use.

### EXAMPLES

### Materials and Methods

### SCF Nucleotide Sequences and Proteins

The human gene encoding Stem Cell Factor (SCF) is also known as kit ligand and has the official symbol KITLG and HGNC number HGNC: 6343. SCF is also known as SF; MGF; SCF; FPH2; KL-1; Kit1; SHEP7; and kit-ligand. Two transcript variants encoding different isoforms have been found for this gene. The SCF (kit ligand) isoform b precursor is available at GenBank accession numbers NM_000899 (mRNA transcript; SEQ ID NO: 3) and NP_000890 (protein sequence; SEQ ID NO: 4). The SCF (kit ligand) isoform a precursor is available at GenBank accession numbers NM_003994 (mRNA transcript; SEQ ID NO: 5) and NP_003985 (protein sequence; SEQ ID NO: 6). The NCBI Reference Gene Sequence has accession number NG_012098 (SEQ ID NO: 7). For both isoforms, the first 25 amino acids comprise the signal peptide and the mature form begins at amino acid 26. The first 11 amino acids of the mature form are EGICRNRVTNN (SEQ ID NO: 8).

### Bleomycin Model

Interstitial pulmonary fibrosis was induced in specific pathogen-free (SPF) female, CBA/J mice (6-8 weeks old; The Jackson Laboratory, Bar Harbor, ME) by the i.t. injection of 0.003 U of bleomycin (Blenoxane, sterile bleomycin sulfate; Bristol-Meyers Pharmaceuticals, Evansville, IN; 0.15 U/Kg of mouse body weight) dissolved in 60 μl of phosphate-buffered saline (PBS). Controls received 60 μl of PBS by the same route. All procedures were conducted in a sterile environment and were approved by the institutional animal care and use committee.

### Whole Lung Histology

Following anesthesia-induced euthanasia, whole lungs from bleomycin-challenged mice were fully inflated with 10% formalin, dissected, and placed in fresh formalin for 24 hours. Routine histological techniques were used to embed the entire lung in paraffin, and 5-μm sections of whole lung were stained with hematoxylin and eosin.

### Production and Administration of Anti-SCF Polyclonal Antibodies

Anti-SCF antibodies were generated by immunizing rabbits with recombinant (whole protein) SCF and generating polyclonal SCF-specific antibodies. Polyclonal antibodies were isolated from the serum using a protein G column. The isolated IgG portion was quantified and used at the specified concentrations suspended in saline. IgG from pre-immune serum was isolated in a similar fashion for use as a control. Briefly, 100, 150 or 200 μg of control or anti-SCF was given to mice by intranasal administration 7 days after treatment with bleomycin. This treatment was repeated on a daily basis until 12 days after bleomycin administration. Thus, the treatment protocol is considered therapeutic.

### Generation of Mouse Anti-Human Monoclonal Antibodies

After identifying an immunogenic human peptide (e.g., SEQ ID NO: 1 or 8), mice were immunized with a standard protocol. The determination of high titer serum antibodies indicated the appropriate immunization and fusion hybridomas were made. Culture supernatants were analyzed from individual clones for SCF-specific antibody and chosen based upon specificity. Five hybridomas producing specific monoclonal antibodies against the peptide were propagated and the monoclonal with the highest titer was subsequently tested in biologically relevant cultures. In some embodiments, a peptide having the sequence EGICRNRVTNN (SEQ ID NO: 8) was used to generate an antibody (e.g., a monoclonal antibody). In some embodiments, any peptide fragment (e.g., an antigenic fragment) of the SCF protein sequence (e.g., as provided by SEQ ID NO: 4 and/or SEQ ID NO: 6) is used to generate antibodies. In some embodiments, mutant or variant forms (e.g., comprising one or more amino acid substitutions with respect to the sequences provided by SEQ ID NO: 4 and SEQ ID NO: 6) of SCF are used to provide a peptide for generating antibodies. It is to be understood that these embodiments comprise additions, deletions, substitutions, post-translational modifications (e.g., glycosylation, cyclization, N- and C-terminal modification, etc.) and other variations of proteins and peptides that are known in the art of molecular biology as applied to provide a peptide for antibody generation.

### Testing Mouse Anti-Human Monoclonal Antibodies

To demonstrate that monoclonal antibodies inhibit SCF, mast cell lines that are sensitive to SCF were tested. The HMC-1 cell line, a mastocytoma cell line that expresses c-Kit and responds to SCF was first used. In brief, HMC-1 cells were cultured in specific growth media and plated in 24-well tissue culture plates at a concentration of 1×10⁶ cells/ml. Recombinant human SCF (1-100 ng/ml) was mixed with monoclonal anti-SCF antibody (12 μg/ml) and incubated at 37° C. for 30 minutes. After incubation, the antibody/SCF or SCF alone was added to the HMC-1 cells. After 1 hour or 24 hours, the cultured HMC-1 cells were harvested and mRNA and protein levels were measured as an indication of SCF inhibition by the monoclonal antibodies.

Analysis of mRNA Expression by Quantitative PCR

Cells or tissue to be tested were dispersed in 1 ml of Trizol reagent (Invitrogen). RNA was isolated as described (Invitrogen), and 5 μg of mRNA was reverse-transcribed to assess gene expression. Detection of cytokine mRNA was determined using previously available primer/probe sets (PE Biosystems, Foster City, CA) and analyzed using an ABI Prism 7500 Sequence Detection System (Applied Biosystems, Foster City, CA). GAPDH mRNA was measured as a control for normalizing mRNA expression. Changes in gene expression were calculated relative to gene expression in unchallenged mice.

### Determination of Cytokine Production

Protein levels of cytokines were quantified using a Bio-Plex bead-based cytokine assay purchased from Bio-Rad Laboratories (Hercules, CA). Using standard protocols, the level of cytokines can be quickly and consistently assessed with this methodology.

### Statistical Analysis

Data were analyzed using Prism GraphPad software. Unless otherwise specified, data shown are representative of two or more experiments. Statistical significance in all experiments was determined by one-way ANOVA, followed by a Newman-Keuls post test. Significant differences were regarded as p<0.05.

Isolation and Propagation of Pulmonary Fibroblasts from Patient Populations

The Institutional Review Board at the University of Michigan Medical School approved this study. All patients underwent clinical evaluation, including chest radiography, lung function measurements, and thin-section computed tomography before fiber optic bronchoscopy. In these patients, interstitial pneumonia was determined from a compilation of symptoms, physiological symptoms, and radiographical findings. Surgical lung biopsies were obtained via the Clinical Core at the University of Michigan Medical School from patients suspected of having interstitial pneumonia between May 2000 and May 2002. Histologically normal lung was obtained from resected specimens in patients undergoing thoracic resection. Each biopsy was processed separately using sterile technique in a laminar flow hood and processed for culturing primary fibroblast lines. Two pathologists who were unaware of any other clinical findings independently reviewed each biopsy and histological classification was based on previously published criteria for idiopathic interstitial pneumonia.

Interstitial pneumonia and normal biopsies were finely minced and the dispersed tissue pieces were placed into 150-cm² cell culture flasks (Corning Inc., Corning, NY) containing Dulbecco's modified Eagle's medium (DMEM, BioWhittaker, Walkersville, MD) supplemented with 15% fetal bovine serum (DMEM-15, BioWhittaker), 1 mmol/L glutamine (BioWhittaker), 100 U/ml penicillin (BioWhittaker), 100 μg/ml streptomycin (BioWhittaker), and 0.25 μg amphotericin B (Fungizone; BioWhittaker). All primary lung cell lines were maintained in DMEM-15 at 37° C. in a 5% CO₂ incubator and were serially passaged a total of five times to yield pure populations of lung fibroblasts. All primary fibroblast cell lines were used at passages 6 to 10 in the experiments outlined below and all of the experiments were performed under comparable conditions.

### 1. Anti-SCF Antibody Reduces Fibrosis and Inflammation

Experiments conducted while developing embodiments of the technology demonstrated that anti-SCF antibody reduced fibrosis and inflammation. Pulmonary fibrosis was induced in mice as described. On day 7 following bleomycin injury, mice were subjected to treatment with anti-SCF antibodies delivered into the airway by intranasal administration. Treatment continued until day 12 following bleomycin exposure. Lungs were harvested on day 16 and examined by microscopy and a series of micrographs were taken. Lung histology demonstrated that anti-SCF antibodies reduced overall inflammation. In addition, Masson's trichrome staining, which designates collagen deposition, was reduced.

### 2. Anti-SCF Antibody Reduces Levels of SCF, Hydroxyproline, IL-25, and IL-13

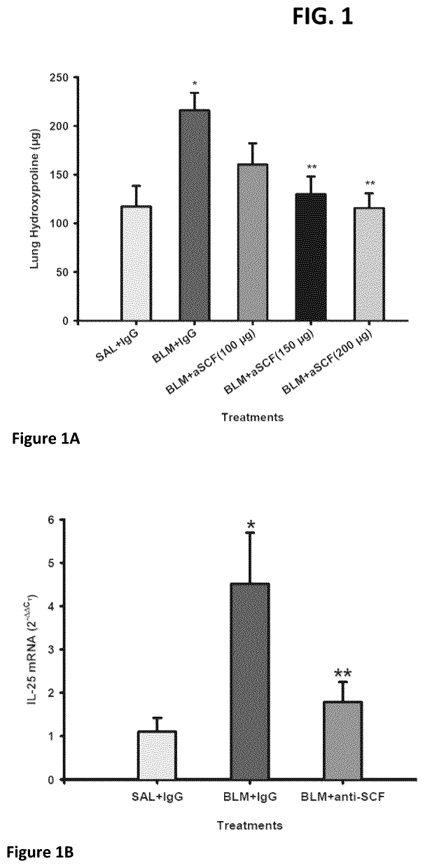

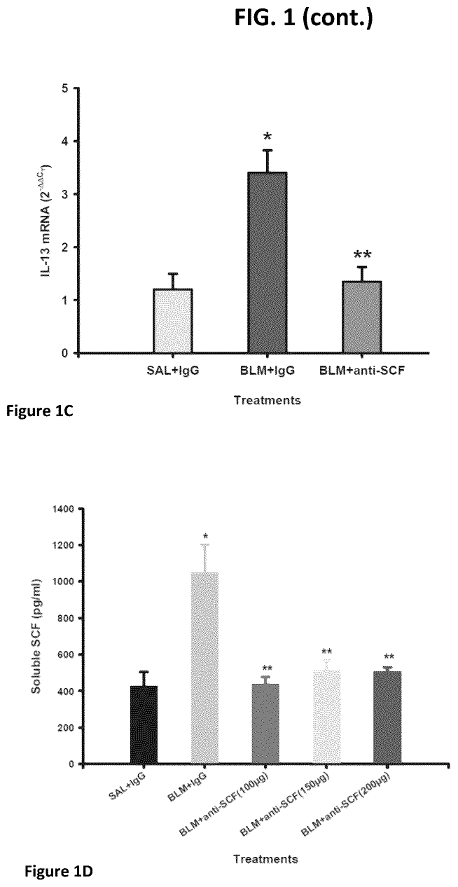

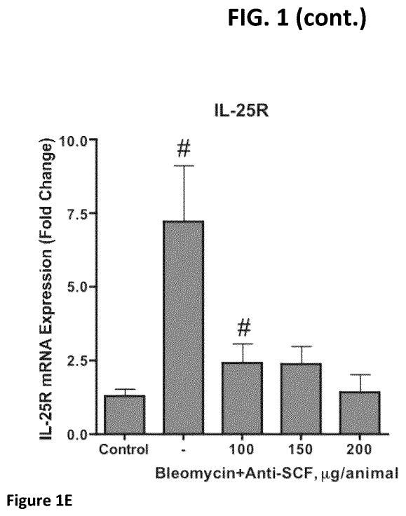

Levels of hydroxyproline and particular cytokines were monitored while developing embodiments of the technology. Lung tissue sections from the above experiment were examined for the presence of hydroxyproline, a collagen precursor. The data demonstrated that the anti-SCF antibody reduced the production of hydroxyproline and plasma levels of SCF in a dose-dependent manner (FIGS. 1A and D). Also, IL-25 and IL-13 expression, measured as a function of mRNA levels, were reduced, as was expression of IL-25 receptor (FIGS. 1B, C, and E).

In particular, the experiments tested the effect of anti-SCF antibody treatment in the BLM model (FIG. 1). Mice were treated with saline (FIG. 1, “SAL”) or BLM (FIG. 1, “BLM”) on day 0. On days 8 and 12, different groups were also treated intratracheally with non-immune (FIG. 1, “IgG”) or anti-SCF antibodies (FIG. 1, “aSCF”) at the indicated doses. H&E stained lung tissue sections from each treatment group were acquired and examined. Fibrosis was quantified biochemically as lung hydroxyproline content (FIG. 1A). Lungs were then analyzed for IL-13 mRNAs by real time PCR (FIG. 1C). Plasma and lung tissue collected from SAL- or BLM-treated mice were then analyzed for soluble SCF by ELISA (FIG. 1D) or IL-25 mRNA by real time PCR (FIG. 1B). Values represent the means+/−the standard error with an n=7. A single asterisk (*) indicates statistical significance (P<0.05) when compared to the saline control group, while double asterisks (**) indicate significance with respect to the BLM+IgG control group.

3. IL-4 Stimulates c-Kit Expression in Human Fibroblasts

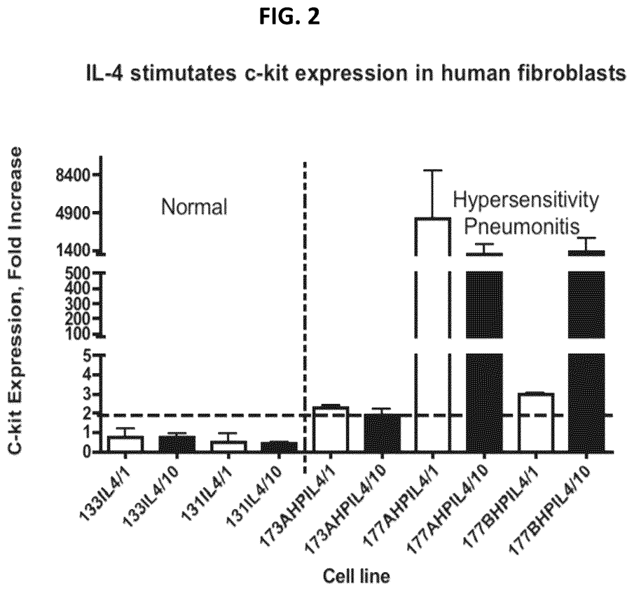

Experiments conducted while developing embodiments of the technology demonstrated that IL-4 stimulated c-kit expression in human fibroblasts. In addition to the mouse model of pulmonary inflammation, SCF receptor is expressed in fibroblast populations from patients diagnosed with hypersensitivity pneumonitis and who thus have a pro-fibrotic environment. Pulmonary fibroblasts were grown from normal areas of lungs from patients (normal) and those diagnosed with hypersensitivity pneumonitis. Expression of c-kit was measured after stimulation with IL-4 at 1 or 10 ng/ml. Individual cell lines (133, 131, 173, 177A, 177B) were assessed using real-time PCR. Compared to lung fibroblasts grown from patients with non-fibrotic disease, fibroblasts from the hypersensitivity pneumonitis patients displayed significant upregulation of c-kit when stimulated with IL-4, a fibrosis-associated cytokine. The data demonstrated that SCF activated fibroblasts from inflammatory lesions, but not those from normal tissue, and promoted the expression of fibrosis-associated genes including collagen (FIG. 2).

### 4. A Mouse Anti-Human Monoclonal Antibody Blocks SCF-Induced HMC Mast Cell Activation.

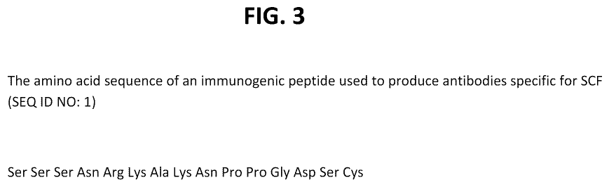

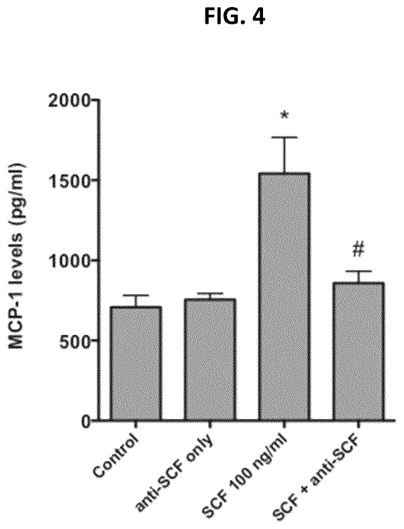

Experiments conducted while developing embodiments of the technology demonstrated that the monoclonal antibody specific for SCF inhibited the activation of HMC-1 cells for MCP-1 production. The activation of mast cells is a classic SCF-induced response that can be used to monitor antibody neutralization of SCF-mediated cytokine responses. Previous studies have demonstrated that monocyte chemotactic protein (MCP)-1 is strongly upregulated by SCF in mast cells. A monoclonal antibody was produced against SEQ ID NO: 1 (FIG. 3). The efficacy of this antibody was tested using a human mast cell line, HMC-1, stimulated with 100 ng/ml of SCF. The monoclonal antibody (6 μg/ml) was preincubated with the recombinant SCF for 5 minutes prior to placing the SCF or the SCF plus anti-SCF onto the cultured HMC-1 cells (1×10⁶ cells/ml). The cells were subsequently incubated for 12 hours, after which the cell-free supernatant was collected and MCP-1 was analyzed by Bio-Plex. The data illustrate that the monoclonal antibody specific for SCF inhibited the activation of HMC-1 cells for MCP-1 production (FIG. 4).

5. SCF-Deficient Mice Subjected to BLM-Induced Injury have Reduced Fibrosis.

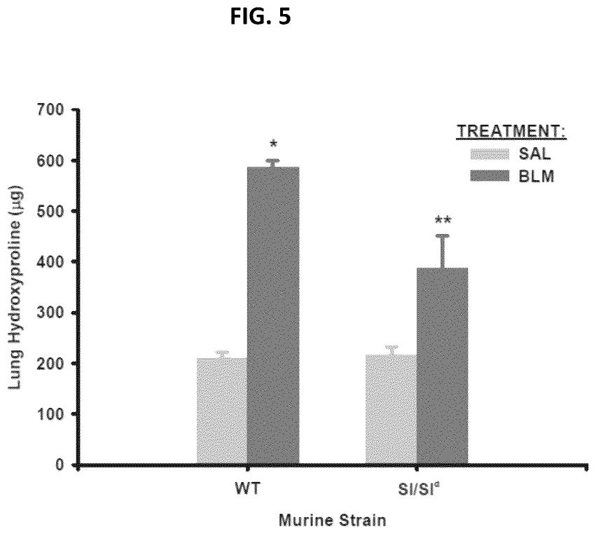

During the development of embodiments of the technology provided herein, the effects of SCF deficiency in Kit1^{S1}/Kitl^{S1-d} mutant mice were examined (FIG. 5). These mice have a complete deletion of the SCF gene in one allele (S1) and a deletion of the membrane-bound ligand in the other (S1^{d}), which significantly decreases the expression of soluble SCF. When these mice and their wild type controls (WT) were subjected to BLM-induced lung injury, there was a significantly reduced fibrosis in the mutant mice compared to wild type mice, both morphologically (Masson trichrome stain) and biochemically by hydroxyproline analysis (FIG. 5).

Wild-type and SCF deficient mice were treated with saline (“SAL”) or BLM (“BLM”) on day 0 and lungs were harvested 21 days later. Fibrosis was quantified biochemically as lung hydroxyproline content. Values represent the mean+/−standard deviation with an n=3. A single asterisk (*) indicates statistical significance (P<0.05) when compared to the WT saline-treated control mean, while double asterisks (**) indicate significance when compared to the WT BLM-treated group.

Similar suppression of cytokine expression and telomerase induction was also noted in S1/S1d mice. These data taken together indicated an essential role for the SCF/c-Kit signaling induced pulmonary fibrosis.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

1-18. (canceled)

19. A peptide comprising an amino acid sequence provided by SEQ ID NO: 1 conjugated to an immunogenic carrier.

20. The peptide of claim 19, wherein the immunogenic carrier comprises diphtheria toxoid, bovine serum albumin (BSA), or keyhole limpet hemocyanin (KLH).

21. A nucleic acid comprising a nucleotide sequence encoding a peptide comprising an amino acid sequence provided by SEQ ID NO: 1.

22. The nucleic acid of claim 21, wherein said nucleic acid comprises a nucleotide sequence provided by SEQ ID NO: 2.

23. The nucleic acid of claim 21, wherein said nucleic acid comprises a promoter.

24. The nucleic acid of claim 21, wherein said nucleic acid is an expression vector.

25. A cell comprising a recombinant nucleic acid comprising a nucleotide sequence encoding a peptide comprising an amino acid sequence provided by SEQ ID NO: 1.

26. The cell of claim 25, wherein said recombinant nucleic acid comprises a nucleotide sequence provided by SEQ ID NO: 2.

27. The cell of claim 25, wherein said cell is a bacterial expression system.
